SEQUENCE LISTING (456 sequences)

SEQ ID NO: 1 - protein, 92 aa, synthetic construct
  REGION 1..92: note = synthetic construct
SSSSSEKLRETQAQVDEVVDIMRVNVDKVLERDQKLSELDDRADALQAGASQFETSAAKLKRKYWWKNLKMMIILGVICAIILIIIIVYFST

SEQ ID NO: 2 - protein, 92 aa, synthetic construct
  REGION 1..92: note = synthetic construct
NLASNRRLQQTSEEVREVNDIMRVNVDKVLERDQKLSELDDRADALQAGASQFETSAAKLKRKYWWKNLKMMIILGVICAIILIIIIVYFST

SEQ ID NO: 3 - protein, 92 aa, synthetic construct
  REGION 1..92: note = synthetic construct
NLASNRRLQQTQAQVDEVVDVMRDNRNLVDERDQKLSELDDRADALQAGASQFETSAAKLKRKYWWKNLKMMIILGVICAIILIIIIVYFST

SEQ ID NO: 4 - protein, 92 aa, synthetic construct
  REGION 1..92: note = synthetic construct
NLASNRRLQQTQAQVDEVVDIMRVNVDKVLRQGEQIDRLEDRADALQAGASQFETSAAKLKRKYWWKNLKMMIILGVICAIILIIIIVYFST

SEQ ID NO: 5 - protein, 92 aa, synthetic construct
  REGION 1..92: note = synthetic construct
NLASNRRLQQTQAQVDEVVDIMRVNVDKVLERDQKLSELDERADELEKSASQFETSAAKLKRKYWWKNLKMMIILGVICAIILIIIIVYFST

SEQ ID NO: 6 - protein, 92 aa, synthetic construct
  REGION 1..92: note = synthetic construct
NLASNRRLQQTQAQVDEVVDIMRVNVDKVLERDQKLSELDDRADALQAGAERLEENATKLKRKYWWKNLKMMIILGVICAIILIIIIVYFST

SEQ ID NO: 7 - protein, 92 aa, synthetic construct
  REGION 1..92: note = synthetic construct
NLASNRRLQQTQAQVDEVVDIMRVNVDKVLERDQKLSELDDRADALQAGASQFETSAAKLTKYYEEKESKMMIILGVICAIILIIIIVYFST

SEQ ID NO: 8 - protein, 92 aa, synthetic construct
  REGION 1..92: note = synthetic construct
SSSSNEKLRETLREVEDVKNIMEDNRRLVERQGRQIDRLEEKADDLERSAERLSDNSEKLKRKYWWKNSKMMIILGVICAIILIIIIVYFST

SEQ ID NO: 9 - protein, 92 aa, synthetic construct
  REGION 1..92: note = synthetic construct
SSSDSSKLRETEEETRDVIDIMRDNRRSVEERGRQIDRLEERADDLEDSAERLEENAKKLKRKYWWKNSKMMIILGVICAIILIIIIVYFST

SEQ ID NO: 10 - protein, 92 aa, synthetic construct; identical to SEQ ID NO: 8
  REGION 1..92: note = synthetic construct

SEQ ID NO: 11 - protein, 92 aa, synthetic construct
  REGION 1..92: note = synthetic construct
SGSSNERLREVSKEAREVREMAMDVKEKIEEQGRKIEELEEKAESLKDSAERFDENAKKLKRKYWWKNLKMMIILGVICAIILIIIIVYFST

SEQ ID NO: 12 - protein, 92 aa, synthetic construct
  REGION 1..92: note = synthetic construct
SGTTNEKLRKVSSEADEVKEMGMDVKEKVEEQGRKIEELEEKAEDLKDSAERFDENAKKLKRKYWWKNLKMMIILGVICAIILIIIIVYFST

SEQ ID NO: 13 - protein, 92 aa, synthetic construct
  REGION 1..92: note = synthetic construct
SGSSSEKLRQISSEAEEVKEMGMDILKKIEEQGEKIERLEEKAESLKDSAERFADNAKKLKRKYWWKNLKMMIILGVICAIILIIIIVYFST

SEQ ID NO: 14 - protein, 92 aa, synthetic construct
  REGION 1..92: note = synthetic construct
DGTSNERLRETSKEAREVRDMAMDNMKKVEEQGEKIEELEEKAEELKDSAERLDDNAKKLKRKYWWKNLKMMIILGVICAIILIIIIVYFST

SEQ ID NO: 15 - protein, 92 aa, synthetic construct
  REGION 1..92: note = synthetic construct
DGTSNEKLRETSEQAREVRDMALDNKEKIEEQGEKIDRLEEKAESLKDSAERFAENAKKLKRKYWWKNLKMMIILGVICAIILIIIIVYFST

SEQ ID NO: 16 - protein, 92 aa, synthetic construct
  REGION 1..92: note = synthetic construct
SEEMSKKLEETSKEVDEVLEIMEEIREMLEEQGRRIDRLEKKAEELEEGAEKFEELSEKLKRKYWWKNLKMMIILGVICAIILIIIIVYFST

SEQ ID NO: 17 - protein, 92 aa, synthetic construct
  REGION 1..92: note = synthetic construct
SEERKEKLEETLKEVDEVLEIMKENKEMLEEQGERLERLEEKAEELEEGAEKFEELAEKLKRKYWWKNLKMMIILGVICAIILIIIIVYFST

SEQ ID NO: 18 - protein, 92 aa, synthetic construct
  REGION 1..92: note = synthetic construct
SKERSEKLKETMEEVEEVLEIMKEIRRMMEEQGERIDRLEEKAEELEEGAEKFEELAEKLKRKYWWKNLKMMIILGVICAIILIIIIVYFST

SEQ ID NO: 19 - protein, 92 aa, synthetic construct
  REGION 1..92: note = synthetic construct
SSSSNEKLRETLREVEDVKNIMEDNRRLVERQGRQIDRLEEKADDLERSAERLSDNSEKLKRKYWWKNLKMMIILGVICAIILIIIIVYFST

SEQ ID NO: 20 - protein, 92 aa, synthetic construct
  REGION 1..92: note = synthetic construct
SSSSNEKLRETLREVEDVKNIMEDNRRLVERQGRQIDRLEEKADDLERSAERLSDNSEKLKKKKKKKKKKMMIILGVICAIILIIIIVYFST

SEQ ID NO: 21 - protein, 93 aa, synthetic construct
  REGION 1..93: note = synthetic construct
SSSSNEKLRETLREVEDVKNIMEDNRRLVERQGRQIDRLEEKADDLERSAERLSDNSEKLKRKYWWKNLKAAVLVLLVIVIISLIVLVVIWST

SEQ ID NO: 22 - protein, 92 aa, synthetic construct
  REGION 1..92: note = synthetic construct
SSSSNEKLRETLREVEDVKNIMEDNRRLVERQGRQIDRLEEKADDLERSAERLSDNSEKLKRKYWWKNLKMMVVVVVVVVVVVVVVVVYFST

SEQ ID NO: 23 - protein, 92 aa, synthetic construct
  REGION 1..92: note = synthetic construct
SSSSNEKLRETLREVEDVKNIMEDNRRLVERQGRQIDRLEEKADDLERSAERLSDNSEKLKRKYWWKNLKMMIIIIIIIIIIIIIIIIYFST

SEQ ID NO: 24 - protein, 93 aa, synthetic construct
  REGION 1..93: note = synthetic construct
SSSSNEKLRETLREVEDVKNIMEDNRRLVERQGRQIDRLEEKADDLERSAERLSDNSEKLKKKKKKKKKKAAVLVLLVIVIISLIVLVVIWST

SEQ ID NO: 25 - protein, 92 aa, synthetic construct
  REGION 1..92: note = synthetic construct
SSSSNEKLRETLREVEDVKNIMEDNRRLVERQGRQIDRLEEKADDLERSAERLSDNSEKLKKKKKKKKKKMMVVVVVVVVVVVVVVVVYFST

SEQ ID NO: 26 - protein, 92 aa, synthetic construct
  REGION 1..92: note = synthetic construct
NLASNRRLQQTQAQVDEVVDIMRVNVDKVLERDQKLSELDDRADALQAGASQFETSAAKLKKKKKKKKKKMMIILGVICAIILIIIIVYFST

SEQ ID NO: 27 - protein, 92 aa, synthetic construct
  REGION 1..92: note = synthetic construct
NLASNRRLQQTQAQVDEVVDIMRVNVDKVLERDQKLSELDDRADALQAGASQFETSAAKLKRKYWWKNLKMMLLLLLLLLLLLLLLLLYFST

SEQ ID NO: 28 - protein, 92 aa, synthetic construct
  REGION 1..92: note = synthetic construct
NLASNRRLQQTQAQVDEVVDIMRVNVDKVLERDQKLSELDDRADALQAGASQFETSAAKLKRKYWWKNLKMMVVVVVVVVVVVVVVVVYFST

SEQ ID NO: 29 - protein, 93 aa, synthetic construct
  REGION 1..93: note = synthetic construct
NLASNRRLQQTQAQVDEVVDIMRVNVDKVLERDQKLSELDDRADALQAGASQFETSAAKLKRKYWWKNLKFFFIIGLIIGLFLVLRVGIHLST

SEQ ID NO: 30 - protein, 93 aa, synthetic construct
  REGION 1..93: note = synthetic construct
NLASNRRLQQTQAQVDEVVDIMRVNVDKVLERDQKLSELDDRADALQAGASQFETSAAKLKRKYWWKNLKILWISFAISCFLLCVVLLGFIST

SEQ ID NO: 31 - protein, 94 aa, synthetic construct
  REGION 1..94: note = synthetic construct
NLASNRRLQQTQAQVDEVVDIMRVNVDKVLERDQKLSELDDRADALQAGASQFETSAAKLKRKYWWKNLKIATGMVGALLLLLVVALGIGLFST

SEQ ID NO: 32 - protein, 93 aa, synthetic construct
  REGION 1..93: note = synthetic construct
NLASNRRLQQTQAQVDEVVDIMRVNVDKVLERDQKLSELDDRADALQAGASQFETSAAKLKRKYWWKNLKIMIIICCVILGIVIASTVGGIST

SEQ ID NO: 33 - protein, 93 aa, synthetic construct
  REGION 1..93: note = synthetic construct
NLASNRRLQQTQAQVDEVVDIMRVNVDKVLERDQKLSELDDRADALQAGASQFETSAAKLKRKYWWKNLKAAVLVLLVIVIISLIVLVVIWST

SEQ ID NO: 34 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKFFFKKFKMMIILGVICAIILIIIIVYFST

SEQ ID NO: 35 - protein, 92 aa, synthetic construct
  REGION 1..92: note = synthetic construct
SSSSNEKLRETLREVEDVKNIMEDNRRLVERQGRQIDRLEEKADDLERSAERLSDNSERIRRRFFFRRFRMMIILGVICAIILIIIIVYFST

SEQ ID NO: 36 - protein, 93 aa, synthetic construct
  REGION 1..93: note = synthetic construct
SSSSNEKLRETLREVEDVKNIMEDNRRLVERQGRQIDRLEEKADDLERSAERLSDNSEKIKKKFFFKKFKAAVLVLLVIVIISLIVLVVIWST

SEQ ID NO: 37 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKFFFKKFKMMVVVVVVVVVVVVVVVVYFST

SEQ ID NO: 38 - protein, 205 aa, synthetic construct
  REGION 1..205: note = synthetic construct
AEDADLEKQKQEEEKRGETLKDESLEATRKMVNMVREAREMAMRNGELLESQGEKLDRIEEKADRMETKLDEADEDLKKIEGFSGLSVSPSNKLKSSDAYKKAWGNNQDGVVASQPARVVDEREQMAISGGFIRRVTNDARENEMDENLEQVSGIIGNLRHMALDMGNEIDTQNRQIDRIMEKADSAKTRIDEANQRATKMLGSG

SEQ ID NO: 39 - protein, 205 aa, synthetic construct
  REGION 1..205: note = synthetic construct
AEDADSLAQQQQEEQRGSTLIDESLEATRKMKEMVEEAVRMAMDNGELLRSQGEKLDRIEEKADRMESLLDEADENLDKIEGFSGLSVSPSNKLKSSDAYKKAWGNNQDGVVASQPARVVDEREQMAISGGFIRRVTNDARENEMDENLEQVSGIIGNLRHMALDMGNEIDTQNRQIDRIMEKADSAKTRIDEANQRATKMLGSG

SEQ ID NO: 40 - protein, 205 aa, synthetic construct
  REGION 1..205: note = synthetic construct
AEDADMRNELEEMQRRADQLADESLESTRRMLQLVEESKDAGIRTLVMLDEQGEQLERIEEGMDQINKDMKEAEKNLADLGKFSGLSVSPSNKLKSSDAYKKAWGNNQDGVVASQPARVVDEREQMAISPSYIREVNNSEKEKEINEGLGRVDQQVQELKDMAVVMGEKVDEQNEKIDRINEKADKNEQRVNDLTKEAEKLLNSG

SEQ ID NO: 41 - protein, 205 aa, synthetic construct
  REGION 1..205: note = synthetic construct
AEDADMRNELEEMQRRADQLADESLESTRRMLQLVEESKDAGIRTLVMLDEQGEQLERIEEGMDQINKDMKEAEKNLADLGKFSGLSVSPSNKLKSSDAYKKAWGNNQDGVVASQPARVVDEREQMAISSSFIRRVNGSEREREIDRGLERVDQQVKELKDMARVMGDKTDEQGEKIDRIEEKADRNEERVEKLVKEAKELLESG

SEQ ID NO: 42 - protein, 155 aa, synthetic construct
  REGION 1..155: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEAEEEE

SEQ ID NO: 43 - protein, 155 aa, synthetic construct
  REGION 1..155: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEK

SEQ ID NO: 44 - protein, 155 aa, synthetic construct
  REGION 1..155: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEK

SEQ ID NO: 45 - protein, 103 aa, synthetic construct
  REGION 1..103: note = synthetic construct
SIEMEELSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 46 - protein, 103 aa, synthetic construct
  REGION 1..103: note = synthetic construct
SISKQALKRLEERHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 47 - protein, 103 aa, synthetic construct
  REGION 1..103: note = synthetic construct
SISKQALSEIETNNEQVIKLENSIRELHDMFMDMAMLVESQGEMIDRIEYNVEHAVDYVERAVSDTKKAVKYQSKARRKKIMIIICCVILGIVIASTVGGIFA

SEQ ID NO: 48 - protein, 103 aa, synthetic construct
  REGION 1..103: note = synthetic construct
SISKQALSEIETRHSEIRRLLESIRELHDMFMDMAMLVESQGEMIDRIEYNVEHAVDYVERAVSDTKKAVKYQSKARRKKIMIIICCVILGIVIASTVGGIFA

SEQ ID NO: 49 - protein, 103 aa, synthetic construct
  REGION 1..103: note = synthetic construct
SISKQALSEIETRHSEIIKLENSVEEMHDMFMDMAMLVESQGEMIDRIEYNVEHAVDYVERAVSDTKKAVKYQSKARRKKIMIIICCVILGIVIASTVGGIFA

SEQ ID NO: 50 - protein, 103 aa, synthetic construct
  REGION 1..103: note = synthetic construct
SISKQALSEIETRHSEIIKLENSIRELKDMARDMAMLVESQGEMIDRIEYNVEHAVDYVERAVSDTKKAVKYQSKARRKKIMIIICCVILGIVIASTVGGIFA

SEQ ID NO: 51 - protein, 103 aa, synthetic construct
  REGION 1..103: note = synthetic construct
SISKQALSEIETRHSEIIKLENSIRELHDMFMRLGDMVESQGEMIDRIEYNVEHAVDYVERAVSDTKKAVKYQSKARRKKIMIIICCVILGIVIASTVGGIFA

SEQ ID NO: 52 - protein, 103 aa, synthetic construct
  REGION 1..103: note = synthetic construct
SISKQALSEIETRHSEIIKLENSIRELHDMFMDMAMLVDEQGEMIDRIEYNVEHAVDYVERAVSDTKKAVKYQSKARRKKIMIIICCVILGIVIASTVGGIFA

SEQ ID NO: 53 - protein, 103 aa, synthetic construct
  REGION 1..103: note = synthetic construct
SISKQALSEIETRHSEIIKLENSIRELHDMFMDMAMLVESQGEKISRIEYNVEHAVDYVERAVSDTKKAVKYQSKARRKKIMIIICCVILGIVIASTVGGIFA

SEQ ID NO: 54 - protein, 103 aa, synthetic construct
  REGION 1..103: note = synthetic construct
SISKQALSEIETRHSEIIKLENSIRELHDMFMDMAMLVESQGEMIDRIEERVEHAVDYVERAVSDTKKAVKYQSKARRKKIMIIICCVILGIVIASTVGGIFA

SEQ ID NO: 55 - protein, 103 aa, synthetic construct
  REGION 1..103: note = synthetic construct
SISKQALSEIETRHSEIIKLENSIRELHDMFMDMAMLVESQGEMIDRIEYNVEAAEAYVERAVSDTKKAVKYQSKARRKKIMIIICCVILGIVIASTVGGIFA

SEQ ID NO: 56 - protein, 103 aa, synthetic construct
  REGION 1..103: note = synthetic construct
SISKQALSEIETRHSEIIKLENSIRELHDMFMDMAMLVESQGEMIDRIEYNVEHAVDGVKAAVSDTKKAVKYQSKARRKKIMIIICCVILGIVIASTVGGIFA

SEQ ID NO: 57 - protein, 103 aa, synthetic construct
  REGION 1..103: note = synthetic construct
SISKQALSEIETRHSEIIKLENSIRELHDMFMDMAMLVESQGEMIDRIEYNVEHAVDYVERAKDNTKKAVKYQSKARRKKIMIIICCVILGIVIASTVGGIFA

SEQ ID NO: 58 - protein, 103 aa, synthetic construct; identical to SEQ ID NO: 57
  REGION 1..103: note = synthetic construct

SEQ ID NO: 59 - protein, 103 aa, synthetic construct
  REGION 1..103: note = synthetic construct
SISKQALSEIETRHSEIIKLENSIRELHDMFMDMAMLVESQGEMIDRIEYNVEHAVDYVERAVSDTKKAVKYQSKARRKKFFFIIGLIIGLFLVLRVGIHLFA

SEQ ID NO: 60 - protein, 103 aa, synthetic construct
  REGION 1..103: note = synthetic construct
SISKQALSEIETRHSEIIKLENSIRELHDMFMDMAMLVESQGEMIDRIEYNVEHAVDYVERAVSDTKKAVKYQSKARRKKILWISFAISCFLLCVVLLGFIFA

SEQ ID NO: 61 - protein, 104 aa, synthetic construct
  REGION 1..104: note = synthetic construct
SISKQALSEIETRHSEIIKLENSIRELHDMFMDMAMLVESQGEMIDRIEYNVEHAVDYVERAVSDTKKAVKYQSKARRKKIATGMVGALLLLLVVALGIGLFFA

SEQ ID NO: 62 - protein, 102 aa, synthetic construct
  REGION 1..102: note = synthetic construct
SISKQALSEIETRHSEIIKLENSIRELHDMFMDMAMLVESQGEMIDRIEYNVEHAVDYVERAVSDTKKAVKYQSKARRKKMMIILGVICAIILIIIIVYFFA

SEQ ID NO: 63 - protein, 103 aa, synthetic construct
  REGION 1..103: note = synthetic construct
SISKQALSEIETRHSEIIKLENSIRELHDMFMDMAMLVESQGEMIDRIEYNVEHAVDYVERAVSDTKKAVKYQSKARRKKAAVLVLLVIVIISLIVLVVIWFA

SEQ ID NO: 64 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEAEEEEGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 65 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 66 - protein, 262 aa, synthetic construct
  REGION 1..262: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 67 - protein, 257 aa, synthetic construct
  REGION 1..257: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 68 - protein, 252 aa, synthetic construct
  REGION 1..252: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 69 - protein, 247 aa, synthetic construct
  REGION 1..247: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 70 - protein, 242 aa, synthetic construct
  REGION 1..242: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 71 - protein, 257 aa, synthetic construct
  REGION 1..257: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEGGSGGSGGSALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 72 - protein, 252 aa, synthetic construct
  REGION 1..252: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEGGSGGSGGSALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 73 - protein, 247 aa, synthetic construct
  REGION 1..247: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKGGSGGSGGSALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 74 - protein, 242 aa, synthetic construct
  REGION 1..242: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELGGSGGSGGSALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 75 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKEELAKVEERHKQIQALLDKIEELYEMFKEMSEKISEQGQKIDRIEEKVSKASEHVSKGVEDTKKAVKYQSKARRKKIMIIICCVILGIVIASTVGGIFA

SEQ ID NO: 76 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEVEKRHKQILELEEKIKELYEMFKEMSEKIEKQGQKIDRIDDKVSEAKKHVEKAVEDTKKAVKYQSKARRKKIMIIICCVILGIVIASTVGGIFA

SEQ ID NO: 77 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 78 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEAVKKSLAAKEERHKQILELLEKIKELHEMFKELSEKIEKQGQKIDRIEDKVSKASEHVSKGVEDTKKAVKYQSKARRKKIMIIICCVILGIVIASTVGGIFA

SEQ ID NO: 79 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEAVKKELAAIEERHEQILELLKKIEELYEMFKELSEKIEKQGQKIDRIEKKVSEASRHVSKAVEDTKKAVKYQSKARRKKIMIIICCVILGIVIASTVGGIFA

SEQ ID NO: 80 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKDLAAIEERHQQILELEEKIKELHEMFKEMSEKISEQMQKIDRIEEKVSKASEHVSKGVEDTKKAVKYQSKARRKKIMIIICCVILGIVIASTVGGIFA

SEQ ID NO: 81 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVRRELAAIEERHRQILELLEKIEELHEMFKEMSEKISKQMEKIDRIDDRVSEASRHVEKGVEDTKKAVKYQSKARRKKIMIIICCVILGIVIASTVGGIFA

SEQ ID NO: 82 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKAVKEELANIENRHKQIDALYEKIKELHEMFLEMSERIEAQLQKIDRIDDKVSKAKAHVEKGVEDTKKAVKYQSKARRKKIMIIICCVILGIVIASTVGGIFA

SEQ ID NO: 83 - protein, 267 aa, synthetic construct
  REGION 1..267: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 84 - protein, 262 aa, synthetic construct
  REGION 1..262: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 85 - protein, 267 aa, synthetic construct
  REGION 1..267: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSDTKKAVKYQSKARRKKLLLLLLLLLLLLLLLLLLLLLFA

SEQ ID NO: 86 - protein, 266 aa, synthetic construct
  REGION 1..266: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSDTKKAVKYQSKARRKKMMIILGVICAIILIIIIVYFFA

SEQ ID NO: 87 - protein, 266 aa, synthetic construct
  REGION 1..266: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSDTKKAVKYQSRRRRRRMMIILGVICAIILIIIIVYFFA

SEQ ID NO: 88 - protein, 267 aa, synthetic construct
  REGION 1..267: note = synthetic construct
NEREKEIDEGLERVGELISKLKELAREMSEKIEEQNQKLSEIDKKAEEAIKLLEKANASAKKLLEKPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 89 - protein, 267 aa, synthetic construct
  REGION 1..267: note = synthetic construct
NEREKEIEEGLERVGELISELKEMAREMSEKIEEQNKKLDEISKKADEAIKLLEKANKGAEELLKKPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 90 - protein, 267 aa, synthetic construct
  REGION 1..267: note = synthetic construct
NEREKEIDEGLEKIGELISKLKEMAREMSEKIEEQNEKLDEIDKKADEAIKLLEEANKKAEKLLKKKGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 91 - protein, 267 aa, synthetic construct
  REGION 1..267: note = synthetic construct
NEREKEIEEGLERIGELISKLKELAREMSEKIEEQNEKLSEISEKADEAIKLLEKANASAQKLLEKPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 92 - protein, 267 aa, synthetic construct
  REGION 1..267: note = synthetic construct
NPREEEIDKGLEEIGKLISELKELAREMSEKIEEQNEKISEIDEKAKEAIELLKKANEKAKELLEKEGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 93 - protein, 267 aa, synthetic construct
  REGION 1..267: note = synthetic construct
SPREKEIDEGLERVSELVKKLKELAEKMKEMIEEQGRRIERIERKAEEAKERIEKLNEKAEKLLEDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 94 - protein, 267 aa, synthetic construct
  REGION 1..267: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 95 - protein, 267 aa, synthetic construct
  REGION 1..267: note = synthetic construct
SEREKEIDEGLEKVSEIVKELKEMAEEMREMIERQGEQIERIEKKAEEAKKKIEEQNERAERLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 96 - protein, 267 aa, synthetic construct
  REGION 1..267: note = synthetic construct
SEREKEIEEGLERVSEIVRRLKELAEEMRRMIEEQGRRIDRIEEKADKAKEEIEKQNEKLEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 97 - protein, 267 aa, synthetic construct
  REGION 1..267: note = synthetic construct
SEREKEIDEGLEKVSEIVKELKELAKEMKEMIEEQGRRIDRIERKAEETKKKIEELNEQAERLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 98 - protein, 267 aa, synthetic construct
  REGION 1..267: note = synthetic construct
SEREEEIDKGLERVSEIVKKLKELAEKMKEEIERQGEQIDRIEKKADETIKEIERLNESADRLLKSPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 99 - protein, 267 aa, synthetic construct
  REGION 1..267: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSRRRRRRIMIIICCVILGIVIASTVGGIFA

SEQ ID NO: 100 - protein, 267 aa, synthetic construct
  REGION 1..267: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKKKKKKIMIIICCVILGIVIASTVGGIFA

SEQ ID NO: 101 - protein, 265 aa, synthetic construct
  REGION 1..265: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKAAVLVLLVIVIISLIVLVVIW

SEQ ID NO: 102 - protein, 264 aa, synthetic construct
  REGION 1..264: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKMMVVVVVVVVVVVVVVVVYF

SEQ ID NO: 103 - protein, 264 aa, synthetic construct
  REGION 1..264: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKMMIIIIIIIIIIIIIIIIYF

SEQ ID NO: 104 - protein, 265 aa, synthetic construct
  REGION 1..265: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSRRRRRRAAVLVLLVIVIISLIVLVVIW

SEQ ID NO: 105 - protein, 264 aa, synthetic construct
  REGION 1..264: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSRRRRRRMMVVVVVVVVVVVVVVVVYF

SEQ ID NO: 106 - protein, 265 aa, synthetic construct
  REGION 1..265: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKKKKKKAAVLVLLVIVIISLIVLVVIW

SEQ ID NO: 107 - protein, 264 aa, synthetic construct
  REGION 1..264: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKKKKKKMMVVVVVVVVVVVVVVVVYF

SEQ ID NO: 108 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKESLANKEERHKQILELEEKIKELYEMFKELSEKIEEQLKKIDRIEEKVSEASRHVSKGVESLKEAVEYDEKARRKKIMIIICCVILGIVIASTVGGIFA

SEQ ID NO: 109 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKTRRTLAEIEERHRQILELEEKIEELYEMFKELSEKISEQGQKISRIEDKVSKASEHVSKGVENLKKAVEYDEKARRKKIMIIICCVILGIVIASTVGGIFA

SEQ ID NO: 110 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKSLAEIEKRHEQILQLEKQIEELHEMFKELSEKISKQGQKIDRIEEKVEEAKRHVEKAVKDLKEAVEYEEKARRKKIMIIICCVILGIVIASTVGGIFA

SEQ ID NO: 111 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKKVKEELARIEARHQQILALEEKIRELYEMFKELSEKIEEQGKKIDRIEDKVSKASEHVSKGVENLKEAVEYDEKARRKKIMIIICCVILGIVIASTVGGIFA

SEQ ID NO: 112 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKKVKEELKEKEKRHRQIEELLKKIEELHEMFEELSERISEQGQKIDRIDDKVSKASEHVSKGVEDLKEAVEYEEKARRKKIMIIICCVILGIVIASTVGGIFA

SEQ ID NO: 113 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEEVKKSLAEIEKRHEQILALEKKIEELYEMFKELGEKIEKQLQKISRIEEKVSEASRHVSKGVEDLKEAVKYREESEKMEIMIIICCVILGIVIASTVGGIFA

SEQ ID NO: 114 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEIEARHQQIEALLEQIKELYEMFKELSEKIEEQGQKISRIEDKVSKASEHVSKGVEQLKEAVKYNEEGKKMEIMIIICCVILGIVIASTVGGIFA

SEQ ID NO: 115 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEKEKRHKQIDELLEKIKELYEMFKEMGEKIEKQGEKIDRIEKKVSEASKHVSKAVEDLKEAVEYREKSEKMEIMIIICCVILGIVIASTVGGIFA

SEQ ID NO: 116 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEAVKKELAAIEARHKQIDALLEKIKELHEMFEEMSKKIEEQMQKISRIEDKVSEASRHVSKAVSDLKEAVKYKEESEKMEIMIIICCVILGIVIASTVGGIFA

SEQ ID NO: 117 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEIEKRHKQILELEEKIKELHEMFKELGEKIEKQGQKISRIDDKVSEAKRHVEKGVEDLKKAVEYKEKSEKKEIMIIICCVILGIVIASTVGGIFA

SEQ ID NO: 118 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKKVKEELKKIEERHKQILELEEKIEELYEMFKELAERIEKQGEKIDRIDEKVSEAKRNVEKAVEDTKKAVKYQSESEKMEIMIIICCVILGIVIASTVGGIFA

SEQ ID NO: 119 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELEEKEKRHKQILELEEKIKELYEMFKELSEKIEEQLQKIDRIDDKVSEASRHVSKGVEDTKKAVKYQSEAEKMEIMIIICCVILGIVIASTVGGIFA

SEQ ID NO: 120 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEEVRRSLEEIERRHRQILELEEKIEELYEMFKEMSEKIEEQGQKISRIEEKVSKASEHVSKAVEDTKKAVKYQSEAEKKEIMIIICCVILGIVIASTVGGIFA

SEQ ID NO: 121 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEEVKKELAEIEARHEQIKELEKQIEELHEMFKELGEKIEKQGEKIDRIDEKVSEASRHVSKAVEDTKKAVKYQSESEKMEIMIIICCVILGIVIASTVGGIFA

SEQ ID NO: 122 - protein, 267 aa, synthetic construct
  REGION 1..267: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKEELAKKEARHEQILELTEKIEELSEMFKELSEKISEQGQKIDRIEDKVSKASEHVSKAVEDTKKAVKYQSESEKMEIMIIICCVILGIVIASTVGGIFA

SEQ ID NO: 123 - protein, 247 aa, synthetic construct
  REGION 1..247: note = synthetic construct
SELEKKIDELLEEISRLVRELKEIAKELRRLTERQGRQVERIEREVEEAEREIEELNKEAEELLEKEDSEDELEKLKKLLEESKEQLREVERIEREVRRLREEQRRLLELTERAARLAEEALEKMEKMLELQEKILESMKEPDKPYTPEELEKVKERHELIKKLKEEIKELKEMFEELRELVRRQGERLDRIEEKVRRAVEHVKKAEENIGEAVKYLEKSKELEIMIIICCVILGIVIASTVGGIFA

SEQ ID NO: 124 - protein, 247 aa, synthetic construct
  REGION 1..247: note = synthetic construct
SEKEKEIDELLDKVSEIVKELKKLAEELKRRTERQGRQIEEIERKTEEAKRKIEELNKKAEELLKKEDDDSDLEKTKELLKEAKEQLREVKEIKRRVEELKREQEETLKLTKEAAELAEEAKELMEEMLELSEEILEEMLENPKPYTPEELEKVRERHELIKKLLEEIEELEEMFEELERLVEEQGRRLERIEEKVSRAVRHVERAEENLGEAVEYLEKSKKLEIMIIICCVILGIVIASTVGGIFA

SEQ ID NO: 125 - protein, 247 aa, synthetic construct
  REGION 1..247: note = synthetic construct
SSKEEEIEELLDEVSEIVRRLKEMAREIREMVERQGRQIERIERKVEEAKRKIEELNKKAEELLEKEDDESELEELEKLVEEAKEQLREVEEINREVEELGREQERLLRKTREAAKLAEKAEELMKKMLELSEEILEEMKEKPKEYTPEELEEVEERHKLIQKLLEEIKELKEMFEELERLVEEQGRRLERIEEKVRRAVEHVKRALENLKEAKEYRKKNEELEIMIIICCVILGIVIASTVGGIFA

SEQ ID NO: 126 - protein, 244 aa, synthetic construct
  REGION 1..244: note = synthetic construct
SELEKKIDELLEEISRLVRELKEIAKELRRLTERQGRQVERIEREVEEAEREIEELNKEAEELLEKEDSEDELEKLKKLLEESKEQLREVERIEREVRRLREEQRRLLELTERAARLAEEALEKMEKMLELQEKILESMKEPDKPYTPEELEKVKERHELIKKLKEEIKELKEMFEELRELVRRQGERLDRIEEKVRRAVEHVKKAEENIGEAVKYLEKSKELEMMVVVVVVVVVVVVVVVVYF

SEQ ID NO: 127 - protein, 244 aa, synthetic construct
  REGION 1..244: note = synthetic construct
SEKEKEIDELLDKVSEIVKELKKLAEELKRRTERQGRQIEEIERKTEEAKRKIEELNKKAEELLKKEDDDSDLEKTKELLKEAKEQLREVKEIKRRVEELKREQEETLKLTKEAAELAEEAKELMEEMLELSEEILEEMLENPKPYTPEELEKVRERHELIKKLLEEIEELEEMFEELERLVEEQGRRLERIEEKVSRAVRHVERAEENLGEAVEYLEKSKKLEMMVVVVVVVVVVVVVVVVYF

SEQ ID NO: 128 - protein, 267 aa, synthetic construct
  REGION 1..267: note = synthetic construct
SETEKKFNEALEELERLLEEAKKLLEEQRRLLEAQGEVQKEQEKLFDELEEIQEEAEKYQNKLLESKDEEDEMSLLKKALELLEKASKLLEELEALLKKQKELLEKQKELMKELEEVLKKIEEKLKKIKELQEEELKEKKKELAEAEAAEKAGKAAGVSLKEEVEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 129 - protein, 267 aa, synthetic construct
  REGION 1..267: note = synthetic construct
SSLEKKIDENLEKALELLEELKEKLEEMRRLLEESGRLQDELEELMDETQKQQEELEKLLEKLLKMDDSDEQYELLKEALKKQKELKEQLEELEEKLKELRRAHEETRRKMEEAEELLKELEEVMEELKKAQEELLKEKKKKYEEAKKELEEAKKKGEEGKEKLEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 130 - protein, 267 aa, synthetic construct
  REGION 1..267: note = synthetic construct
SEEKKKMEELLDKIKELLEELKKLAEEIKKLLEEQGRQLEKLEEEADKALRQAEEAIRLQEKALELEDDEEIDEALKELEEKQKKLKEQLEKLEEQISELKKLFEEQKKKMEEAEELLKEMLELIKEMKENHEKLLEEAKKRYEEKLKEYEELKKLGILPKEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 131 - protein, 267 aa, synthetic construct
  REGION 1..267: note = synthetic construct
EEFEKKENELLEELKKKLEEAKKLLKENRRLLEEQGRQLEEIEEKMEEAEELQEKALEYQEKAEKAGFSDESFEYLKEALKVLEELEEQLEEIEEKLEEQRELLEKQRELLKEAEKKLKEAEEVCKKLKELIEKRKEEAEEKLKKAEEKAKEAAKKGVDLSEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 132 - protein, 267 aa, synthetic construct
  REGION 1..267: note = synthetic construct
SEEEEKFNELLEKIEEELEEIKELAEELREKLEELGRLTEKALELADELEKLFEEAEKLLEEALKLGDGEELEEVLKEALEKLKEAKEKLEKLEEELSKLKEAQEEAKELLEELEEELKELEEEIKKLKELSEETLKKAKEKLKEAEKKAEELKKLGIDPTEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 133 - protein, 267 aa, synthetic construct
  REGION 1..267: note = synthetic construct
SSLKEKFNELLDELLKELEEAKELLEEIREQLERIGEQLEELEEQFDEILKEQEELEKQQKKLLESPGSEEEEEQLKEIEEKQKKIKEKIEELEEQIEELKEQQEKLKELTKELEEKLKEISETLKELKKVQEELLKEKKKKYEEAKKKYEEKKKKGINGTEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 134 - protein, 267 aa, synthetic construct
  REGION 1..267: note = synthetic construct
KESEEKFEEKLKELEKLLEEAKELLEKQREYLEESGRLLDEAEKLMDETERIFEETLKLQDKLLAAKDEEDQMSLLKKALELLEKASKLLDELEATLKELKALLEKQKELMEELEKVLKEIEEKMKEIKKLQEEELKEQKKKLKEAEEKEKEGEKKGVSYKEEVEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 135 - protein, 267 aa, synthetic construct
  REGION 1..267: note = synthetic construct
SKLKEEFEKYLEELLKQLEKLKEKLKELREKLEEQGKQLEKLEEQFDRILEQQEKLLEQQEKLLEDEGSEEEEELLKEIEKQQEKLKEEIEKLEEQIKKLKEQQEELKEISEKAKELLKKTAEILKKLKEVQEKLLEEKKKELEEAEKKYEELKKKGINGTEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 136 - protein, 267 aa, synthetic construct
  REGION 1..267: note = synthetic construct
SEREKEFNERLEEMEKLLEKIKKLAEEIRRLLEKQGELLDKLEELADEALRLQEKAIEKSEKILEKGYNEETEEELKELLKLLKELEELLDEAEELIDEIKRLLEEQKKLMEEMEKALKKLEELTKKLKELIEKELEEQRKRLEELEKRKEEYEKLGIDLSEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 137 - protein, 267 aa, synthetic construct
  REGION 1..267: note = synthetic construct
SSFEEEINKLLEELKRKLEELKEILEEIRKLLEEQGRQLDEIEEKMDEAEELAEKAEEYLKKAEEAGGGEESYEYLKKALETLKELEEKLDEIEEKLSEQKKLLEETREKLEEAEKKLKEAEKVIKKLKELIEKEKKEKEAELKKAEAAAAEAAKLGIDKSEELEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 138 - protein, 267 aa, synthetic construct
  REGION 1..267: note = synthetic construct
SEEEEKFNELLEKIEEKLEEAKELAEELREELEKIGELTDEAERLADEALKLAEEAEKLLKEALKLGDEDELDKILKEAEKTLEELKKKLEELEEKLKELKEAQEKAKELLKELEETLKELEELIKELKKFSEETLEKAKEKYKKAEEKYKEDLKKGIDNTEEIEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 139 - protein, 267 aa, synthetic construct
  REGION 1..267: note = synthetic construct
ESFEKELEELLEKIQELMEKIKELAEKLREALEESGRLLEEIEEAVDKLEEKFEEIEKLQENAEKYFDTEEAEKYLKEMEEKLKKAKELLDKLEELVSKLKELQEKQRELMEKLEEKLKELLELLKKLKELIEKLKEKKKKELEEAEKKLKEAEEYNEELEKEVEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 140 - protein, 267 aa, synthetic construct
  REGION 1..267: note = synthetic construct
SELEEKFNKYLEELLETLEKLKEALEKIREKLEEQGKQLDKIEEAFDELLKQQEELLKQQEELLADPGSEESEKKLKEIEKQQEKIKEQIEKLEEQIKKLRELQEKQKELTEKAKELLEKLEEILKKLKEVQEKLLEEKKKEYEEAEKEYKEDKKKGINNKEKLEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 141 - protein, 265 aa, synthetic construct
  REGION 1..265: note = synthetic construct
SSLEKKIDENLEKALELLEELKEKLEEMRRLLEESGRLQDELEELMDETQKQQEELEKLLEKLLKMDDSDEQYELLKEALKKQKELKEQLEELEEKLKELRRAHEETRRKMEEAEELLKELEEVMEELKKAQEELLKEKKKKYEEAKKELEEAKKKGEEGKEKLEKVKRELAQIEERHQQILELEEKIKELLEMFKELSEKIEEQGQKIDRIEDKVSKAKEHVEKGVEDTKKAVKYQSKARRKKAAVLVLLVIVIISLIVLVVIW

SEQ ID NO: 142 - protein, 264 aa, synthetic construct
  REGION 1..264: note = synthetic construct
SSLEKKIDENLEKALELLEELKEKLEEMRRLLEESGRLQDELEELMDETQKQQEELEKLLEKLLKMDDSDEQYELLKEALKKQKELKEQLEELEEKLKELRRAHEETRRKMEEAEELLKELEEVMEELKKAQEELLKEKKKKYEEAKKELEEAKKKGEEGKEKLEKVKRELAQIEERHQQILELEEKIKELLEMFKELSEKIEEQGQKIDRIEDKVSKAKEHVEKGVEDTKKAVKYQSKARRKKMMVVVVVVVVVVVVVVVVYF

SEQ ID NO: 143 - protein, 267 aa, synthetic construct
  REGION 1..267: note = synthetic construct
GELKEKKEKLSKEFEKLLKESKRLAEELKEKLEELGRALDEAEELADEVERQQEELEKLQEEILKSEENEDEKKQLEELEKKLKELEELLKELEEKLKEVEELMKEVEELMEELEKTMEEMEKAIEELEKVYKEELKKTEAKLKATKAEAEAAKAKGEDISDKLEAAEKEYKSVKEELKLVEEIKKKVEEIKEMLEEMKERIEEMEEKVKRIEEKLKRIEESLKRVEENLKEIKELAKKREEKGIMIIICCVILGIVIASTVGGIFG

SEQ ID NO: 144 - protein, 267 aa, synthetic construct
  REGION 1..267: note = synthetic construct
DELEKKIKELEEKSEEELKEAKELAEELRRLLEELERALDEAERLADEVERKQEELEKLMEEMLKSEDNESDEEDLKKLKEKLEELEKLLEELEERAREVEELMERVEETMEELEEEMEELLETLKKLLEVYEELLKKKKKELEETKKKAEEMKKKGIDISEELEKAKEELESVKKNLELVKKILEEVKEIKEELEEMGEEIERMEEKVDRIEEKLERVEESLERVSKNLEEIERLFEERKEKGIMIIICCVILGIVIASTVGGIFG

SEQ ID NO: 145 - protein, 267 aa, synthetic construct
  REGION 1..267: note = synthetic construct
SEEDKKMEELLEEALKLLEELKELLEKNRELLEELGRQQEELEKLQDEAERLQEELEEAFKKMEENEESEEGKKYLEEAEKLLKELKKLLEEIEKKTKEIEELVKKQEELMKKIKEVMKKLEEKMKELYRISKERLERAKEEAARAEAARAEYEAAGSPEVERAEQVLEEYREAKEFYEKVEELLREVKEIKEEIKEMEERIKEIGERIKRIEEKIERVEKLLERTEKNLKEIKELRDKIEKNGIMIIICCVILGIVIASTVGGIFG

SEQ ID NO: 146 - protein, 267 aa, synthetic construct
  REGION 1..267: note = synthetic construct
SEKEKEFNELLEEALRELEKLKELLEENGRLLERTGEQLERMEELMDEAEEKQEELEEAIKKMEKYEDSEEGDEYLEEAEELLEELEELLEEIEAQTEEIEALIKEQEELMKKIKEEMEKLKEAVEKLYEISKEMLEEAKKEYEKAEKAKAEYEAAGKDEVKECEKVKEKYEEAKKRYEQVEKLLKEVEEIKEEIERMGEEIKRQGERIERIEEKIERVEEELERLEENLEEIKKLREKIKENGIMIIICCVILGIVIASTVGGIFN

SEQ ID NO: 147 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTQAQVDEVVDIMRVNVDKVLERDQKLSELDDRADALQAGASQFETSAAKLKRKYWWKNLKMMIILGVICAIILIIIIVYFST

SEQ ID NO: 148 - protein, 367 aa, synthetic construct
  REGION 1..367: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSDARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 149 - protein, 58 aa, synthetic construct
  REGION 1..58: note = synthetic construct
SSSSSEKLRETQAQVDEVVDIMRVNVDKVLERDQKLSELDDRADALQAGASQFETSAA

SEQ ID NO: 150 - protein, 58 aa, synthetic construct
  REGION 1..58: note = synthetic construct
NLASNRRLQQTSEEVREVNDIMRVNVDKVLERDQKLSELDDRADALQAGASQFETSAA

SEQ ID NO: 151 - protein, 58 aa, synthetic construct
  REGION 1..58: note = synthetic construct
NLASNRRLQQTQAQVDEVVDVMRDNRNLVDERDQKLSELDDRADALQAGASQFETSAA

SEQ ID NO: 152 - protein, 58 aa, synthetic construct
  REGION 1..58: note = synthetic construct
NLASNRRLQQTQAQVDEVVDIMRVNVDKVLRQGEQIDRLEDRADALQAGASQFETSAA

SEQ ID NO: 153 - protein, 58 aa, synthetic construct
  REGION 1..58: note = synthetic construct
NLASNRRLQQTQAQVDEVVDIMRVNVDKVLERDQKLSELDERADELEKSASQFETSAA

SEQ ID NO: 154 - protein, 58 aa, synthetic construct
  REGION 1..58: note = synthetic construct
NLASNRRLQQTQAQVDEVVDIMRVNVDKVLERDQKLSELDDRADALQAGAERLEENAT

SEQ ID NO: 155 - protein, 58 aa, synthetic construct
  REGION 1..58: note = synthetic construct
NLASNRRLQQTQAQVDEVVDIMRVNVDKVLERDQKLSELDDRADALQAGASQFETSAA

SEQ ID NO: 156 - protein, 58 aa, synthetic construct
  REGION 1..58: note = synthetic construct
SSSSNEKLRETLREVEDVKNIMEDNRRLVERQGRQIDRLEEKADDLERSAERLSDNSE

SEQ ID NO: 157 - protein, 58 aa, synthetic construct
  REGION 1..58: note = synthetic construct
SSSDSSKLRETEEETRDVIDIMRDNRRSVEERGRQIDRLEERADDLEDSAERLEENAK

SEQ ID NO: 158 - protein, 58 aa, synthetic construct
  REGION 1..58: note = synthetic construct
SGSSNERLREVSKEAREVREMAMDVKEKIEEQGRKIEELEEKAESLKDSAERFDENAK

SEQ ID NO: 159 - protein, 58 aa, synthetic construct
  REGION 1..58: note = synthetic construct
SGTTNEKLRKVSSEADEVKEMGMDVKEKVEEQGRKIEELEEKAEDLKDSAERFDENAK

SEQ ID NO: 160 - protein, 58 aa, synthetic construct
  REGION 1..58: note = synthetic construct
SGSSSEKLRQISSEAEEVKEMGMDILKKIEEQGEKIERLEEKAESLKDSAERFADNAK

SEQ ID NO: 161 - protein, 58 aa, synthetic construct
  REGION 1..58: note = synthetic construct
DGTSNERLRETSKEAREVRDMAMDNMKKVEEQGEKIEELEEKAEELKDSAERLDDNAK

SEQ ID NO: 162 - protein, 58 aa, synthetic construct
  REGION 1..58: note = synthetic construct
DGTSNEKLRETSEQAREVRDMALDNKEKIEEQGEKIDRLEEKAESLKDSAERFAENAK

SEQ ID NO: 163 - protein, 58 aa, synthetic construct
  REGION 1..58: note = synthetic construct
SEEMSKKLEETSKEVDEVLEIMEEIREMLEEQGRRIDRLEKKAEELEEGAEKFEELSE

SEQ ID NO: 164 - protein, 58 aa, synthetic construct
  REGION 1..58: note = synthetic construct
SEERKEKLEETLKEVDEVLEIMKENKEMLEEQGERLERLEEKAEELEEGAEKFEELAE

SEQ ID NO: 165 - protein, 58 aa, synthetic construct
  REGION 1..58: note = synthetic construct
SKERSEKLKETMEEVEEVLEIMKEIRRMMEEQGERIDRLEEKAEELEEGAEKFEELAE

SEQ ID NO: 166 - protein, 58 aa, synthetic construct
  REGION 1..58: note = synthetic construct
SSSSNEKLRETSREVEEVNDIMRDNRNLVDRQGEQIDRLEERADELKDSAERLSENSK

SEQ ID NO: 167 - protein, 58 aa, synthetic construct
  REGION 1..58: note = synthetic construct
SSSSNEKLRETQAQVDEVVDIMRVNVDKVLERDQKLSELDDRADALQAGASQFETSAA

SEQ ID NO: 168 - protein, 58 aa, synthetic construct
  REGION 1..58: note = synthetic construct
NLASNRRLQQTLREVEDVKNIMRVNVDKVLERDQKLSELDDRADALQAGASQFETSAA

SEQ ID NO: 169 - protein, 58 aa, synthetic construct
  REGION 1..58: note = synthetic construct
NLASNRRLQQTQAQVDEVVDIMEDNRRLVEERDQKLSELDDRADALQAGASQFETSAA

SEQ ID NO: 170 - protein, 58 aa, synthetic construct
  REGION 1..58: note = synthetic construct
NLASNRRLQQTQAQVDEVVDIMRVNVDKVLRQGRQIDRLEDRADALQAGASQFETSAA

SEQ ID NO: 171 - protein, 58 aa, synthetic construct
  REGION 1..58: note = synthetic construct
NLASNRRLQQTQAQVDEVVDIMRVNVDKVLERDQKLSELDEKADDLERSASQFETSAA

SEQ ID NO: 172 - protein, 58 aa, synthetic construct
  REGION 1..58: note = synthetic construct
NLASNRRLQQTQAQVDEVVDIMRVNVDKVLERDQKLSELDDRADALQAGAERLSDNSE

SEQ ID NO: 173 - protein, 58 aa, synthetic construct
  REGION 1..58: note = synthetic construct
NLASNRRLQQTLREVEDVKNIMEDNRRLVERQGRQIDRLEEKADDLERSAERLSDNSE

SEQ ID NO: 174 - protein, 58 aa, synthetic construct
  REGION 1..58: note = synthetic construct
SSSSNEKLRETQAQVDEVVDIMEDNRRLVERQGRQIDRLEEKADDLERSAERLSDNSE

SEQ ID NO: 175 - protein, 58 aa, synthetic construct
  REGION 1..58: note = synthetic construct
SSSSNEKLRETLREVEDVKNIMRVNVDKVLRQGRQIDRLEEKADDLERSAERLSDNSE

SEQ ID NO: 176 - protein, 58 aa, synthetic construct
  REGION 1..58: note = synthetic construct
SSSSNEKLRETLREVEDVKNIMEDNRRLVEERDQKLSELDEKADDLERSAERLSDNSE

SEQ ID NO: 177 - protein, 58 aa, synthetic construct
  REGION 1..58: note = synthetic construct
SSSSNEKLRETLREVEDVKNIMEDNRRLVERQGRQIDRLEDRADALQAGAERLSDNSE

SEQ ID NO: 178 - protein, 58 aa, synthetic construct
  REGION 1..58: note = synthetic construct
SSSSNEKLRETLREVEDVKNIMEDNRRLVERQGRQIDRLEEKADDLERSASQFETSAA

SEQ ID NO: 179 - protein, 65 aa, synthetic construct
  REGION 1..65: note = synthetic construct
SEEEEKKKEELKKKLKEALEEAKKAKELAKKALELAERQGRQIDRLEEKADDLERSAERLSDNSE

SEQ ID NO: 180 - protein, 65 aa, synthetic construct
  REGION 1..65: note = synthetic construct
EEEKEKKKEELKEKAKKALEEAKKTKELAKEALELAERQGRQIDRLEEKADDLERSAERLSDNSE

SEQ ID NO: 181 - protein, 72 aa, synthetic construct
  REGION 1..72: note = synthetic construct
SLEAEKKEKEEKEKKKKILELLKELLEETEELKEEAEEIKREVERQGRQIDRLEEKADDLERSAERLSDNSE

SEQ ID NO: 182 - protein, 72 aa, synthetic construct
  REGION 1..72: note = synthetic construct
ELEEELKKKEEEEKRKEILELLKELLEETEELKEEAEEIKEEVERQGRQIDRLEEKADDLERSAERLSDNSE

SEQ ID NO: 183 - protein, 58 aa, synthetic construct
  REGION 1..58: note = synthetic construct
SSSSNEKARETLREVEDVKNIMEDNRRLVERQGRQIDRLEEKADDLERSAERLSDNSE

SEQ ID NO: 184 - protein, 58 aa, synthetic construct
  REGION 1..58: note = synthetic construct
SSSSNEKLRETAREVEDVKNIMEDNRRLVERQGRQIDRLEEKADDLERSAERLSDNSE

SEQ ID NO: 185 - protein, 58 aa, synthetic construct
  REGION 1..58: note = synthetic construct
SSSSNEKLRETLREAEDVKNIMEDNRRLVERQGRQIDRLEEKADDLERSAERLSDNSE

SEQ ID NO: 186 - protein, 58 aa, synthetic construct
  REGION 1..58: note = synthetic construct
SSSSNEKLRETLREVEDAKNIMEDNRRLVERQGRQIDRLEEKADDLERSAERLSDNSE

SEQ ID NO: 187 - protein, 58 aa, synthetic construct
  REGION 1..58: note = synthetic construct
SSSSNEKLRETLREVEDVKNAMEDNRRLVERQGRQIDRLEEKADDLERSAERLSDNSE

SEQ ID NO: 188 - protein, 58 aa, synthetic construct
  REGION 1..58: note = synthetic construct
SSSSNEKLRETLREVEDVKNIAEDNRRLVERQGRQIDRLEEKADDLERSAERLSDNSE

SEQ ID NO: 189 - protein, 58 aa, synthetic construct
  REGION 1..58: note = synthetic construct
SSSSNEKLRETLREVEDVKNIMEDNRRAVERQGRQIDRLEEKADDLERSAERLSDNSE

SEQ ID NO: 190 - protein, 58 aa, synthetic construct
  REGION 1..58: note = synthetic construct
SSSSNEKLRETLREVEDVKNIMEDNRRLAERQGRQIDRLEEKADDLERSAERLSDNSE

SEQ ID NO: 191 - protein, 58 aa, synthetic construct
  REGION 1..58: note = synthetic construct
SSSSNEKLRETLREVEDVKNIMEDNRRLVERQARQIDRLEEKADDLERSAERLSDNSE

SEQ ID NO: 192 - protein, 58 aa, synthetic construct
  REGION 1..58: note = synthetic construct
SSSSNEKLRETLREVEDVKNIMEDNRRLVERQGRQADRLEEKADDLERSAERLSDNSE

SEQ ID NO: 193 - protein, 58 aa, synthetic construct
  REGION 1..58: note = synthetic construct
SSSSNEKLRETLREVEDVKNIMEDNRRLVERQGRQIDRAEEKADDLERSAERLSDNSE

SEQ ID NO: 194 - protein, 58 aa, synthetic construct
  REGION 1..58: note = synthetic construct
SSSSNEKLRETLREVEDVKNIMEDNRRLVERQGRQIDRLEEKADDAERSAERLSDNSE

SEQ ID NO: 195 - protein, 58 aa, synthetic construct
  REGION 1..58: note = synthetic construct
SSSSNEKLRETLREVEDVKNIMEDNRRLVERQGRQIDRLEEKADDLERSAERASDNSE

SEQ ID NO: 196 - protein, 58 aa, synthetic construct
  REGION 1..58: note = synthetic construct
SSSSNEKARETAREAEDVKNIMEDNRRLVERQGRQIDRLEEKADDLERSAERLSDNSE

SEQ ID NO: 197 - protein, 58 aa, synthetic construct
  REGION 1..58: note = synthetic construct
SSSSNEKARETAREVEDVKNIMEDNRRLVERQGRQIDRLEEKADDLERSAERLSDNSE

SEQ ID NO: 198 - protein, 58 aa, synthetic construct
  REGION 1..58: note = synthetic construct
SSSSNEKARETAREAEDAKNIMEDNRRLVERQGRQIDRLEEKADDLERSAERLSDNSE

SEQ ID NO: 199 - protein, 58 aa, synthetic construct
  REGION 1..58: note = synthetic construct
SSSSNEKARETAREAEDAKNAMEDNRRLVERQGRQIDRLEEKADDLERSAERLSDNSE

SEQ ID NO: 200 - protein, 58 aa, synthetic construct
  REGION 1..58: note = synthetic construct
SSSSNEKARETAREAEDAKNAAEDNRRLVERQGRQIDRLEEKADDLERSAERLSDNSE

SEQ ID NO: 201 - protein, 58 aa, synthetic construct
  REGION 1..58: note = synthetic construct
SSSSNEKARETLREVEDVKNIAEDNRRLVERQGRQIDRAEEKADDLERSAERLSDNSE

SEQ ID NO: 202 - protein, 58 aa, synthetic construct
  REGION 1..58: note = synthetic construct
SSSSNEKLKETLKEVEDVKNIMEDNKKLVEKQGKQIDKLEEKADDLEKSAEKLSDNSE

SEQ ID NO: 203 - protein, 58 aa, synthetic construct
  REGION 1..58: note = synthetic construct
SSSSNEKLEETLEEVEDVKNIMEDNEELVEEQGEQIDELEEKADDLEESAEELSDNSE

SEQ ID NO: 204 - protein, 58 aa, synthetic construct
  REGION 1..58: note = synthetic construct
SSSSNEKLRETLREVEDVKNIMEDNRRLVERQGRQIDRLEEKADDLEKSAERLSDNSE

SEQ ID NO: 205 - protein, 58 aa, synthetic construct
  REGION 1..58: note = synthetic construct
SSSSNEKLRETLREVEDVKNIMEDNRRLVERQGRQIDRLEEKADDLEESAERLSDNSE

SEQ ID NO: 206 - protein, 58 aa, synthetic construct
  REGION 1..58: note = synthetic construct
SSSSNEKLRETLREVEDVKNIMEDNRRLVERQGRQIDRLEEKADDLERTAERLSDNSE

SEQ ID NO: 207 - protein, 58 aa, synthetic construct
  REGION 1..58: note = synthetic construct
SSSSNEKLRETLREVEDVKNIMEDNRRLVERQGRQIDRLEEKADDLERAAERLSDNSE

SEQ ID NO: 208 - protein, 58 aa, synthetic construct
  REGION 1..58: note = synthetic construct
SSSSNEKLRETLREVEDVKNIMEDNRRLVERQGRQIDRLEEKADDLEREAERLSDNSE

SEQ ID NO: 209 - protein, 12 aa, synthetic construct
  REGION 1..12: note = synthetic construct
KLKKKKKKKKKK

SEQ ID NO: 210 - protein, 12 aa, synthetic construct
  REGION 1..12: note = synthetic construct
KIKKKFFFKKFK

SEQ ID NO: 211 - protein, 12 aa, synthetic construct
  REGION 1..12: note = synthetic construct
RIRRRFFFRRFR

SEQ ID NO: 212 - protein, 12 aa, synthetic construct
  REGION 1..12: note = synthetic construct
KLTKYYEEKESK

SEQ ID NO: 213 - protein, 12 aa, synthetic construct
  REGION 1..12: note = synthetic construct
KLKRKYWWKNLK

SEQ ID NO: 214 - protein, 12 aa, synthetic construct
  REGION 1..12: note = synthetic construct
KLKRKYWWKNSK

SEQ ID NO: 215 - protein, 12 aa, synthetic construct
  REGION 1..12: note = synthetic construct
KIKKKFFFKKSK

SEQ ID NO: 216 - protein, 12 aa, synthetic construct
  REGION 1..12: note = synthetic construct
KIKKKWWWKKSK

SEQ ID NO: 217 - protein, 12 aa, synthetic construct
  REGION 1..12: note = synthetic construct
KIKKKYYYKKSK

SEQ ID NO: 218 - protein, 12 aa, synthetic construct
  REGION 1..12: note = synthetic construct
KIKKKSSSKKSK

SEQ ID NO: 219 - protein, 12 aa, synthetic construct
  REGION 1..12: note = synthetic construct
KIKKKYYYKKFK

SEQ ID NO: 220 - protein, 12 aa, synthetic construct
  REGION 1..12: note = synthetic construct
KIKKKSSSKKFK

SEQ ID NO: 221 - protein, 12 aa, synthetic construct
  REGION 1..12: note = synthetic construct
KIKKKWWWKKFK

SEQ ID NO: 222 - protein, 12 aa, synthetic construct
  REGION 1..12: note = synthetic construct
KLKKYYEEKQTK

SEQ ID NO: 223 - protein, 22 aa, synthetic construct
  REGION 1..22: note = synthetic construct
MMIILGVICAIILIIIIVYFST

SEQ ID NO: 224 - protein, 23 aa, synthetic construct
  REGION 1..23: note = synthetic construct
FFFIIGLIIGLFLVLRVGIHLST

SEQ ID NO: 225 - protein, 23 aa, synthetic construct
  REGION 1..23: note = synthetic construct
ILWISFAISCFLLCVVLLGFIST

SEQ ID NO: 226 - protein, 24 aa, synthetic construct
  REGION 1..24: note = synthetic construct
IATGMVGALLLLLVVALGIGLFST

SEQ ID NO: 227 - protein, 23 aa, synthetic construct
  REGION 1..23: note = synthetic construct
AAVLVLLVIVIISLIVLVVIWST

SEQ ID NO: 228 - protein, 23 aa, synthetic construct
  REGION 1..23: note = synthetic construct
IMIIICCVILGIVIASTVGGIST

SEQ ID NO: 229 - protein, 22 aa, synthetic construct
  REGION 1..22: note = synthetic construct
MMVVVVVVVVVVVVVVVVYFST

SEQ ID NO: 230 - protein, 22 aa, synthetic construct
  REGION 1..22: note = synthetic construct
MMIIIIIIIIIIIIIIIIYFST

SEQ ID NO: 231 - protein, 22 aa, synthetic construct
  REGION 1..22: note = synthetic construct
MMLLLLLLLLLLLLLLLLYFST

SEQ ID NO: 232 - protein, 21 aa, synthetic construct
  REGION 1..21: note = synthetic construct
MMIILGVICAIILIIIIVYFS

SEQ ID NO: 233 - protein, 23 aa, synthetic construct
  REGION 1..23: note = synthetic construct
IMIIICCVILGIVIASTVGGIFA

SEQ ID NO: 234 - protein, 23 aa, synthetic construct
  REGION 1..23: note = synthetic construct
AAVLVLLVIVIISLIVLVVIWFA

SEQ ID NO: 235 - protein, 23 aa, synthetic construct
  REGION 1..23: note = synthetic construct
SATAATAPPAAPAGEGGPPAPPP

SEQ ID NO: 236 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKFFFKKSKMMIILGVICAIILIIIIVYFST

SEQ ID NO: 237 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKWWWKKSKMMIILGVICAIILIIIIVYFST

SEQ ID NO: 238 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKYYYKKSKMMIILGVICAIILIIIIVYFST

SEQ ID NO: 239 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKSSSKKSKMMIILGVICAIILIIIIVYFST

SEQ ID NO: 240 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKYYYKKFKMMIILGVICAIILIIIIVYFST

SEQ ID NO: 241 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKSSSKKFKMMIILGVICAIILIIIIVYFST

SEQ ID NO: 242 - protein, 92 aa, synthetic construct
  REGION 1..92: note = synthetic construct
SSSSNEKLRETLREVEDVKNIMEDNRRLVERQGRQIDRLEEKADDLERSAERLSDNSEKIKKKWWWKKFKMMIILGVICAIILIIIIVYFST

SEQ ID NO: 243 - protein, 115 aa, synthetic construct
  REGION 1..115: note = synthetic construct
SATAATAPPAAPAGEGGPPAPPPSSSSNEKLRETSREVEEVNDIMRDNRNLVDRQGEQIDRLEERADELKDSAERLSENSKKLKKYYEEKQTKMMIILGVICAIILIIIIVYFST

SEQ ID NO: 244 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETQAQVDEVVDIMRVNVDKVLERDQKLSELDDRADALQAGASQFETSAAKLKRKYWWKNLKMMIILGVICAIILIIIIVYFST

SEQ ID NO: 245 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTLREVEDVKNIMRVNVDKVLERDQKLSELDDRADALQAGASQFETSAAKLKRKYWWKNLKMMIILGVICAIILIIIIVYFST

SEQ ID NO: 246 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTQAQVDEVVDIMEDNRRLVEERDQKLSELDDRADALQAGASQFETSAAKLKRKYWWKNLKMMIILGVICAIILIIIIVYFST

SEQ ID NO: 247 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTQAQVDEVVDIMRVNVDKVLRQGRQIDRLEDRADALQAGASQFETSAAKLKRKYWWKNLKMMIILGVICAIILIIIIVYFST

SEQ ID NO: 248 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTQAQVDEVVDIMRVNVDKVLERDQKLSELDEKADDLERSASQFETSAAKLKRKYWWKNLKMMIILGVICAIILIIIIVYFST

SEQ ID NO: 249 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTQAQVDEVVDIMRVNVDKVLERDQKLSELDDRADALQAGAERLSDNSEKLKRKYWWKNLKMMIILGVICAIILIIIIVYFST

SEQ ID NO: 250 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTQAQVDEVVDIMRVNVDKVLERDQKLSELDDRADALQAGASQFETSAAKLKRKYWWKNSKMMIILGVICAIILIIIIVYFST

SEQ ID NO: 251 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETLREVEDVKNIMEDNRRLVERQGRQIDRLEEKADDLERSAERLSDNSEKLKRKYWWKNSKMMIILGVICAIILIIIIVYFST

SEQ ID NO: 252 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSNLASNRRLQQTLREVEDVKNIMEDNRRLVERQGRQIDRLEEKADDLERSAERLSDNSEKLKRKYWWKNSKMMIILGVICAIILIIIIVYFST

SEQ ID NO: 253 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETQAQVDEVVDIMEDNRRLVERQGRQIDRLEEKADDLERSAERLSDNSEKLKRKYWWKNSKMMIILGVICAIILIIIIVYFST

SEQ ID NO: 254 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETLREVEDVKNIMRVNVDKVLRQGRQIDRLEEKADDLERSAERLSDNSEKLKRKYWWKNSKMMIILGVICAIILIIIIVYFST

SEQ ID NO: 255 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETLREVEDVKNIMEDNRRLVEERDQKLSELDEKADDLERSAERLSDNSEKLKRKYWWKNSKMMIILGVICAIILIIIIVYFST

SEQ ID NO: 256 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETLREVEDVKNIMEDNRRLVERQGRQIDRLEDRADALQAGAERLSDNSEKLKRKYWWKNSKMMIILGVICAIILIIIIVYFST

SEQ ID NO: 257 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETLREVEDVKNIMEDNRRLVERQGRQIDRLEEKADDLERSASQFETSAAKLKRKYWWKNSKMMIILGVICAIILIIIIVYFST

SEQ ID NO: 258 - protein, 205 aa, synthetic construct
  REGION 1..205: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSSSSSNEKLRETLREVEDVKNIMEDNRRLVERQGRQIDRLEEKADDLERSAERLSDNSEKLKRKYWWKNLKMMIILGVICAIILIIIIVYFST

SEQ ID NO: 259 - protein, 98 aa, synthetic construct
  REGION 1..98: note = synthetic construct
SEEEEKKKEELKKKLKEALEEAKKAKELAKKALELAERQGRQIDRLEEKADDLERSAERLSDNSEKLKRKYWWKNSKMMIILGVICAIILIIIIVYFS

SEQ ID NO: 260 - protein, 98 aa, synthetic construct
  REGION 1..98: note = synthetic construct
EEEKEKKKEELKEKAKKALEEAKKTKELAKEALELAERQGRQIDRLEEKADDLERSAERLSDNSEKLKRKYWWKNSKMMIILGVICAIILIIIIVYFS

SEQ ID NO: 261 - protein, 105 aa, synthetic construct
  REGION 1..105: note = synthetic construct
SLEAEKKEKEEKEKKKKILELLKELLEETEELKEEAEEIKREVERQGRQIDRLEEKADDLERSAERLSDNSEKLKRKYWWKNSKMMIILGVICAIILIIIIVYFS

SEQ ID NO: 262 - protein, 105 aa, synthetic construct
  REGION 1..105: note = synthetic construct
ELEEELKKKEEEEKRKEILELLKELLEETEELKEEAEEIKEEVERQGRQIDRLEEKADDLERSAERLSDNSEKLKRKYWWKNSKMMIILGVICAIILIIIIVYFS

SEQ ID NO: 263 - protein, 92 aa, synthetic construct
  REGION 1..92: note = synthetic construct
SSSSNEKARETLREVEDVKNIMEDNRRLVERQGRQIDRLEEKADDLERSAERLSDNSEKLKRKYWWKNSKMMIILGVICAIILIIIIVYFST

SEQ ID NO: 264 - protein, 92 aa, synthetic construct
  REGION 1..92: note = synthetic construct
SSSSNEKLRETAREVEDVKNIMEDNRRLVERQGRQIDRLEEKADDLERSAERLSDNSEKLKRKYWWKNSKMMIILGVICAIILIIIIVYFST

SEQ ID NO: 265 - protein, 92 aa, synthetic construct
  REGION 1..92: note = synthetic construct
SSSSNEKLRETLREAEDVKNIMEDNRRLVERQGRQIDRLEEKADDLERSAERLSDNSEKLKRKYWWKNSKMMIILGVICAIILIIIIVYFST

SEQ ID NO: 266 - protein, 92 aa, synthetic construct
  REGION 1..92: note = synthetic construct
SSSSNEKLRETLREVEDAKNIMEDNRRLVERQGRQIDRLEEKADDLERSAERLSDNSEKLKRKYWWKNSKMMIILGVICAIILIIIIVYFST

SEQ ID NO: 267 - protein, 92 aa, synthetic construct
  REGION 1..92: note = synthetic construct
SSSSNEKLRETLREVEDVKNAMEDNRRLVERQGRQIDRLEEKADDLERSAERLSDNSEKLKRKYWWKNSKMMIILGVICAIILIIIIVYFST

SEQ ID NO: 268 - protein, 92 aa, synthetic construct
  REGION 1..92: note = synthetic construct
SSSSNEKLRETLREVEDVKNIAEDNRRLVERQGRQIDRLEEKADDLERSAERLSDNSEKLKRKYWWKNSKMMIILGVICAIILIIIIVYFST

SEQ ID NO: 269 - protein, 92 aa, synthetic construct
  REGION 1..92: note = synthetic construct
SSSSNEKLRETLREVEDVKNIMEDNRRAVERQGRQIDRLEEKADDLERSAERLSDNSEKLKRKYWWKNSKMMIILGVICAIILIIIIVYFST

SEQ ID NO: 270 - protein, 92 aa, synthetic construct
  REGION 1..92: note = synthetic construct
SSSSNEKLRETLREVEDVKNIMEDNRRLAERQGRQIDRLEEKADDLERSAERLSDNSEKLKRKYWWKNSKMMIILGVICAIILIIIIVYFST

SEQ ID NO: 271 - protein, 92 aa, synthetic construct
  REGION 1..92: note = synthetic construct
SSSSNEKLRETLREVEDVKNIMEDNRRLVERQARQIDRLEEKADDLERSAERLSDNSEKLKRKYWWKNSKMMIILGVICAIILIIIIVYFST

SEQ ID NO: 272 - protein, 92 aa, synthetic construct
  REGION 1..92: note = synthetic construct
SSSSNEKLRETLREVEDVKNIMEDNRRLVERQGRQADRLEEKADDLERSAERLSDNSEKLKRKYWWKNSKMMIILGVICAIILIIIIVYFST

SEQ ID NO: 273 - protein, 92 aa, synthetic construct
  REGION 1..92: note = synthetic construct
SSSSNEKLRETLREVEDVKNIMEDNRRLVERQGRQIDRAEEKADDLERSAERLSDNSEKLKRKYWWKNSKMMIILGVICAIILIIIIVYFST

SEQ ID NO: 274 - protein, 92 aa, synthetic construct
  REGION 1..92: note = synthetic construct
SSSSNEKLRETLREVEDVKNIMEDNRRLVERQGRQIDRLEEKADDAERSAERLSDNSEKLKRKYWWKNSKMMIILGVICAIILIIIIVYFST

SEQ ID NO: 275 - protein, 92 aa, synthetic construct
  REGION 1..92: note = synthetic construct
SSSSNEKLRETLREVEDVKNIMEDNRRLVERQGRQIDRLEEKADDLERSAERASDNSEKLKRKYWWKNSKMMIILGVICAIILIIIIVYFST

SEQ ID NO: 276 - protein, 92 aa, synthetic construct
  REGION 1..92: note = synthetic construct
SSSSNEKLRETLREVEDVKNIMEDNRRLVERQGRQIDRLEEKADDLERSAERLSDNSEKAKRKYWWKNSKMMIILGVICAIILIIIIVYFST

SEQ ID NO: 277 - protein, 92 aa, synthetic construct
  REGION 1..92: note = synthetic construct
SSSSNEKARETAREAEDVKNIMEDNRRLVERQGRQIDRLEEKADDLERSAERLSDNSEKLKRKYWWKNSKMMIILGVICAIILIIIIVYFST

SEQ ID NO: 278 - protein, 92 aa, synthetic construct
  REGION 1..92: note = synthetic construct
SSSSNEKARETAREVEDVKNIMEDNRRLVERQGRQIDRLEEKADDLERSAERLSDNSEKLKRKYWWKNSKMMIILGVICAIILIIIIVYFST

SEQ ID NO: 279 - protein, 92 aa, synthetic construct
  REGION 1..92: note = synthetic construct
SSSSNEKARETAREAEDAKNIMEDNRRLVERQGRQIDRLEEKADDLERSAERLSDNSEKLKRKYWWKNSKMMIILGVICAIILIIIIVYFST

SEQ ID NO: 280 - protein, 92 aa, synthetic construct
  REGION 1..92: note = synthetic construct
SSSSNEKARETAREAEDAKNAMEDNRRLVERQGRQIDRLEEKADDLERSAERLSDNSEKLKRKYWWKNSKMMIILGVICAIILIIIIVYFST

SEQ ID NO: 281 - protein, 92 aa, synthetic construct
  REGION 1..92: note = synthetic construct
SSSSNEKARETAREAEDAKNAAEDNRRLVERQGRQIDRLEEKADDLERSAERLSDNSEKLKRKYWWKNSKMMIILGVICAIILIIIIVYFST

SEQ ID NO: 282 - protein, 92 aa, synthetic construct
  REGION 1..92: note = synthetic construct
SSSSNEKARETLREVEDVKNIAEDNRRLVERQGRQIDRAEEKADDLERSAERLSDNSEKLKRKYWWKNSKMMIILGVICAIILIIIIVYFST

SEQ ID NO: 283 - protein, 92 aa, synthetic construct
  REGION 1..92: note = synthetic construct
SSSSNEKLKETLKEVEDVKNIMEDNKKLVEKQGKQIDKLEEKADDLEKSAEKLSDNSEKLKRKYWWKNSKMMIILGVICAIILIIIIVYFST

SEQ ID NO: 284 - protein, 92 aa, synthetic construct
  REGION 1..92: note = synthetic construct
SSSSNEKLEETLEEVEDVKNIMEDNEELVEEQGEQIDELEEKADDLEESAEELSDNSEKLKRKYWWKNSKMMIILGVICAIILIIIIVYFST

SEQ ID NO: 285 - protein, 92 aa, synthetic construct
  REGION 1..92: note = synthetic construct
SSSSNEKLRETLREVEDVKNIMEDNRRLVERQGRQIDRLEEKADDLEKSAERLSDNSEKLKRKYWWKNSKMMIILGVICAIILIIIIVYFST

SEQ ID NO: 286 - protein, 92 aa, synthetic construct
  REGION 1..92: note = synthetic construct
SSSSNEKLRETLREVEDVKNIMEDNRRLVERQGRQIDRLEEKADDLEESAERLSDNSEKLKRKYWWKNSKMMIILGVICAIILIIIIVYFST

SEQ ID NO: 287 - protein, 92 aa, synthetic construct
  REGION 1..92: note = synthetic construct
SSSSNEKLRETLREVEDVKNIMEDNRRLVERQGRQIDRLEEKADDLERTAERLSDNSEKLKRKYWWKNSKMMIILGVICAIILIIIIVYFST

SEQ ID NO: 288 - protein, 92 aa, synthetic construct
  REGION 1..92: note = synthetic construct
SSSSNEKLRETLREVEDVKNIMEDNRRLVERQGRQIDRLEEKADDLERAAERLSDNSEKLKRKYWWKNSKMMIILGVICAIILIIIIVYFST

SEQ ID NO: 289 - protein, 92 aa, synthetic construct
  REGION 1..92: note = synthetic construct
SSSSNEKLRETLREVEDVKNIMEDNRRLVERQGRQIDRLEEKADDLEREAERLSDNSEKLKRKYWWKNSKMMIILGVICAIILIIIIVYFST

SEQ ID NO: 290 - protein, 107 aa, synthetic construct
  REGION 1..107: note = synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 291 - protein, 94 aa, synthetic construct
  REGION 1..94: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRIS

SEQ ID NO: 292 - protein, 31 aa, synthetic construct
  REGION 1..31: note = synthetic construct
MDSKGSSQKGSRLLLLLVVSNLLLCQGVVST

SEQ ID NO: 293 - protein, 20 aa, synthetic construct
  REGION 1..20: note = synthetic construct
MYRMQLLSCIALSLALVTNS

SEQ ID NO: 294 - protein, 25 aa, synthetic construct
  REGION 1..25: note = synthetic construct
MGVLLTQRTLLSLVLALLFPSMASM

SEQ ID NO: 295 - protein, 16 aa, synthetic construct
  REGION 1..16: note = synthetic construct
MKCLLYLAFLFIGVNC

SEQ ID NO: 296 - protein, 21 aa, synthetic construct
  REGION 1..21: note = synthetic construct
METDTLLLWVLLLWVPGSTGD

SEQ ID NO: 297 - protein, 19 aa, synthetic construct
  REGION 1..19: note = synthetic construct
MGWSCIILFLVATATGVHS

SEQ ID NO: 298 - protein, 17 aa, synthetic construct
  REGION 1..17: note = synthetic construct
MRAWIFFLLCLAGRALA

SEQ ID NO: 299 - protein, 21 aa, synthetic construct
  REGION 1..21: note = synthetic construct
MWWRLWWLLLLLLLLWPMVWA

SEQ ID NO: 300 - protein, 22 aa, synthetic construct
  REGION 1..22: note = synthetic construct
MDMRVPAQLLGLLLLWLRGARC

SEQ ID NO: 301 - protein, 16 aa, synthetic construct
  REGION 1..16: note = synthetic construct
MPLLLLLPLLWAGALA

SEQ ID NO: 302 - protein, 23 aa, synthetic construct
  REGION 1..23: note = synthetic construct
MDAMKRGLCCVLLLCGAVFVSPS

SEQ ID NO: 303 - protein, 18 aa, synthetic construct
  REGION 1..18: note = synthetic construct
MAFLWLLSCWALLGTTFG

SEQ ID NO: 304 - protein, 15 aa, synthetic construct
  REGION 1..15: note = synthetic construct
MNLLLILTFVAAAVA

SEQ ID NO: 305 - protein, 16 aa, synthetic construct
  REGION 1..16: note = synthetic construct
MKPIFLVLLVVTSAYA

SEQ ID NO: 306 - protein, 17 aa, synthetic construct
  REGION 1..17: note = synthetic construct
MGVKVLFALICIAVAEA

SEQ ID NO: 307 - protein, 16 aa, synthetic construct
  REGION 1..16: note = synthetic construct
MKWVTFISLLFSSAYS

SEQ ID NO: 308 - protein, 16 aa, synthetic construct
  REGION 1..16: note = synthetic construct
MKTIIALSYIFCLVLG

SEQ ID NO: 309 - protein, 24 aa, synthetic construct
  REGION 1..24: note = synthetic construct
MALWMRLLPLLALLALWGPDPAAA

SEQ ID NO: 310 - protein, 150 aa, synthetic construct
  REGION 1..150: note = synthetic construct
  VARIANT 83: note = X is any amino acid linker
AEDADLEKQKQEEEKRGETLKDESLEATRKMVNMVREAREMAMRNGELLESQGEKLDRIEEKADRMETKLDEADEDLKKIEGXDARENEMDENLEQVSGIIGNLRHMALDMGNEIDTQNRQIDRIMEKADSAKTRIDEANQRATKMLGSG

SEQ ID NO: 311 - protein, 150 aa, synthetic construct
  REGION 1..150: note = synthetic construct
  VARIANT 83: note = X is any amino acid linker
AEDADSLAQQQQEEQRGSTLIDESLEATRKMKEMVEEAVRMAMDNGELLRSQGEKLDRIEEKADRMESLLDEADENLDKIEGXDARENEMDENLEQVSGIIGNLRHMALDMGNEIDTQNRQIDRIMEKADSAKTRIDEANQRATKMLGSG

SEQ ID NO: 312 - protein, 159 aa, synthetic construct
  REGION 1..159: note = synthetic construct
  VARIANT 83: note = X is any amino acid linker
AEDADMRNELEEMQRRADQLADESLESTRRMLQLVEESKDAGIRTLVMLDEQGEQLERIEEGMDQINKDMKEAEKNLADLGKXPSYIREVNNSEKEKEINEGLGRVDQQVQELKDMAVVMGEKVDEQNEKIDRINEKADKNEQRVNDLTKEAEKLLNSG

SEQ ID NO: 313 - protein, 159 aa, synthetic construct
  REGION 1..159: note = synthetic construct
  VARIANT 83: note = X is any amino acid linker
AEDADMRNELEEMQRRADQLADESLESTRRMLQLVEESKDAGIRTLVMLDEQGEQLERIEEGMDQINKDMKEAEKNLADLGKXSSFIRRVNGSEREREIDRGLERVDQQVKELKDMARVMGDKTDEQGEKIDRIEEKADRNEERVEKLVKEAKELLESG

SEQ ID NO: 314 - protein, 150 aa, synthetic construct
  REGION 1..150: note = synthetic construct
  VARIANT 68: note = X is any amino acid linker
DARENEMDENLEQVSGIIGNLRHMALDMGNEIDTQNRQIDRIMEKADSAKTRIDEANQRATKMLGSGXEELKDLEKEGKELKELVEELDREVKELKESMEKLKEMTEEAAELSSQALEIMRRTRKLSEELLKEAKEEEEEEEEEEAEEEE

SEQ ID NO: 315 - protein, 150 aa, synthetic construct
  REGION 1..150: note = synthetic construct
  VARIANT 68: note = X is any amino acid linker
DARENEMDENLEQVSGIIGNLRHMALDMGNEIDTQNRQIDRIMEKADSAKTRIDEANQRATKMLGSGXEELKKLEKEGEKLKELVEELDREIKELKEGMERLREMFEEAAKLSEEALEIMRRTRKLSEEELEEAKQKAKEQEKEKALKEK

SEQ ID NO: 316 - protein, 150 aa, synthetic construct
  REGION 1..150: note = Synthetic construct
  VARIANT 68: note = X is any amino acid linker
NKREEEIDKGLDRVGEIISKLNEMAREMGEKIEEQNQKISEIEKKADEAIEKVEKLIKDAEKLLGSGXEELKKLEKEGEKLKELVEELDREIKELKEGMERLREMFEEAAKLSEEALEIMRRTRKLSEEELEEAKQKAKEQEKEKALKEK

SEQ ID NO: 317 - protein, 65 aa, synthetic construct
  REGION 1..65: note = synthetic construct
SIEMEELSEIETRHSEIIKLENSIRELHDMFMDMAMLVESQGEMIDRIEYNVEHAVDYVERAVSD

SEQ ID NO: 318 - protein, 65 aa, synthetic construct
  REGION 1..65: note = synthetic construct
SISKQALKRLEERHSEIIKLENSIRELHDMFMDMAMLVESQGEMIDRIEYNVEHAVDYVERAVSD

SEQ ID NO: 319 - protein, 65 aa, synthetic construct
  REGION 1..65: note = synthetic construct
SISKQALSEIETNNEQVIKLENSIRELHDMFMDMAMLVESQGEMIDRIEYNVEHAVDYVERAVSD

SEQ ID NO: 320 - protein, 65 aa, synthetic construct
  REGION 1..65: note = synthetic construct
SISKQALSEIETRHSEIRRLLESIRELHDMFMDMAMLVESQGEMIDRIEYNVEHAVDYVERAVSD

SEQ ID NO: 321 - protein, 65 aa, synthetic construct
  REGION 1..65: note = synthetic construct
SISKQALSEIETRHSEIIKLENSVEEMHDMFMDMAMLVESQGEMIDRIEYNVEHAVDYVERAVSD

SEQ ID NO: 322 - protein, 65 aa, synthetic construct
  REGION 1..65: note = synthetic construct
SISKQALSEIETRHSEIIKLENSIRELKDMARDMAMLVESQGEMIDRIEYNVEHAVDYVERAVSD

SEQ ID NO: 323 - protein, 65 aa, synthetic construct
  REGION 1..65: note = synthetic construct
SISKQALSEIETRHSEIIKLENSIRELHDMFMRLGDMVESQGEMIDRIEYNVEHAVDYVERAVSD

SEQ ID NO: 324 - protein, 65 aa, synthetic construct
  REGION 1..65: note = synthetic construct
SISKQALSEIETRHSEIIKLENSIRELHDMFMDMAMLVDEQGEMIDRIEYNVEHAVDYVERAVSD

SEQ ID NO: 325 - protein, 65 aa, synthetic construct
  REGION 1..65: note = synthetic construct
SISKQALSEIETRHSEIIKLENSIRELHDMFMDMAMLVESQGEKISRIEYNVEHAVDYVERAVSD

SEQ ID NO: 326 - protein, 65 aa, synthetic construct
  REGION 1..65: note = synthetic construct
SISKQALSEIETRHSEIIKLENSIRELHDMFMDMAMLVESQGEMIDRIEERVEHAVDYVERAVSD

SEQ ID NO: 327 - protein, 65 aa, synthetic construct
  REGION 1..65: note = synthetic construct
SISKQALSEIETRHSEIIKLENSIRELHDMFMDMAMLVESQGEMIDRIEYNVEAAEAYVERAVSD

SEQ ID NO: 328 - protein, 65 aa, synthetic construct
  REGION 1..65: note = synthetic construct
SISKQALSEIETRHSEIIKLENSIRELHDMFMDMAMLVESQGEMIDRIEYNVEHAVDGVKAAVSD

SEQ ID NO: 329 - protein, 65 aa, synthetic construct
  REGION 1..65: note = synthetic construct
SISKQALSEIETRHSEIIKLENSIRELHDMFMDMAMLVESQGEMIDRIEYNVEHAVDYVERAKDN

SEQ ID NO: 330 - protein, 65 aa, synthetic construct
  REGION 1..65: note = synthetic construct
SISKQALSEIETRHSEIIKLENSIRELHDMFMDMAMLVESQGEMIDRIEYNVEHAVDYVERAVSD

SEQ ID NO: 331 - protein, 15 aa, synthetic construct
  REGION 1..15: note = synthetic construct
TKKAVKYQSKARRKK

SEQ ID NO: 332 - protein, 15 aa, synthetic construct
  REGION 1..15: note = synthetic construct
TKKAVKYQSRRRRRR

SEQ ID NO: 333 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEAEEEEGGSGGSGGSSISKQALSEIETRHSEIIKLENSIRELHDMFMDMAMLVESQGEMIDRIEYNVEHAVDYVERAVSD

SEQ ID NO: 334 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSD

SEQ ID NO: 335 - protein, 224 aa, synthetic construct
  REGION 1..224: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEGGSGGSGGSSISKQALSEIETRHSEIIKLENSIRELHDMFMDMAMLVESQGEMIDRIEYNVEHAVDYVERAVSD

SEQ ID NO: 336 - protein, 219 aa, synthetic construct
  REGION 1..219: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEGGSGGSGGSSISKQALSEIETRHSEIIKLENSIRELHDMFMDMAMLVESQGEMIDRIEYNVEHAVDYVERAVSD

SEQ ID NO: 337 - protein, 214 aa, synthetic construct
  REGION 1..214: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKGGSGGSGGSSISKQALSEIETRHSEIIKLENSIRELHDMFMDMAMLVESQGEMIDRIEYNVEHAVDYVERAVSD

SEQ ID NO: 338 - protein, 209 aa, synthetic construct
  REGION 1..209: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELGGSGGSGGSSISKQALSEIETRHSEIIKLENSIRELHDMFMDMAMLVESQGEMIDRIEYNVEHAVDYVERAVSD

SEQ ID NO: 339 - protein, 204 aa, synthetic construct
  REGION 1..204: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKGGSGGSGGSSISKQALSEIETRHSEIIKLENSIRELHDMFMDMAMLVESQGEMIDRIEYNVEHAVDYVERAVSD

SEQ ID NO: 340 - protein, 219 aa, synthetic construct
  REGION 1..219: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEEEEEEGGSGGSGGSALSEIETRHSEIIKLENSIRELHDMFMDMAMLVESQGEMIDRIEYNVEHAVDYVERAVSD

SEQ ID NO: 341 - protein, 214 aa, synthetic construct
  REGION 1..214: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKEEEEEGGSGGSGGSALSEIETRHSEIIKLENSIRELHDMFMDMAMLVESQGEMIDRIEYNVEHAVDYVERAVSD

SEQ ID NO: 342 - protein, 209 aa, synthetic construct
  REGION 1..209: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELLKEAKGGSGGSGGSALSEIETRHSEIIKLENSIRELHDMFMDMAMLVESQGEMIDRIEYNVEHAVDYVERAVSD

SEQ ID NO: 343 - protein, 204 aa, synthetic construct
  REGION 1..204: note = synthetic construct
DARENEMDENLEQVSGIIGNLRHMALDMGNEIDTQNRQIDRIMEKADSAKTRIDEANQRATKMLGSGGGSGGSEELKDLEKEGKELKELVEELDREVKELKESMEKLKEMTEEAAELSSQALEIMRRTRKLSEELGGSGGSGGSALSEIETRHSEIIKLENSIRELHDMFMDMAMLVESQGEMIDRIEYNVEHAVDYVERAVSD

SEQ ID NO: 344 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKEELAKVEERHKQIQALLDKIEELYEMFKEMSEKISEQGQKIDRIEEKVSKASEHVSKGVED

SEQ ID NO: 345 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEVEKRHKQILELEEKIKELYEMFKEMSEKIEKQGQKIDRIDDKVSEAKKHVEKAVED

SEQ ID NO: 346 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 347 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEAVKKSLAAKEERHKQILELLEKIKELHEMFKELSEKIEKQGQKIDRIEDKVSKASEHVSKGVED

SEQ ID NO: 348 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEAVKKELAAIEERHEQILELLKKIEELYEMFKELSEKIEKQGQKIDRIEKKVSEASRHVSKAVED

SEQ ID NO: 349 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKDLAAIEERHQQILELEEKIKELHEMFKEMSEKISEQMQKIDRIEEKVSKASEHVSKGVED

SEQ ID NO: 350 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVRRELAAIEERHRQILELLEKIEELHEMFKEMSEKISKQMEKIDRIDDRVSEASRHVEKGVED

SEQ ID NO: 351 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKAVKEELANIENRHKQIDALYEKIKELHEMFLEMSERIEAQLQKIDRIDDKVSKAKAHVEKGVED

SEQ ID NO: 352 - protein, 229 aa, synthetic construct
  REGION 1..229: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSSISKQALSEIETRHSEIIKLENSIRELHDMFMDMAMLVESQGEMIDRIEYNVEHAVDYVERAVSD

SEQ ID NO: 353 - protein, 224 aa, synthetic construct
  REGION 1..224: note = synthetic construct
NKREEEIDKGLDRVGEIISKLNEMAREMGEKIEEQNQKISEIEKKADEAIEKVEKLIKDAEKLLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSALSEIETRHSEIIKLENSIRELHDMFMDMAMLVESQGEMIDRIEYNVEHAVDYVERAVSD

SEQ ID NO: 354 - protein, 229 aa, synthetic construct; identical to SEQ ID NO: 352
  REGION 1..229: note = synthetic construct

SEQ ID NO: 355 - protein, 229 aa, synthetic construct; identical to SEQ ID NO: 352
  REGION 1..229: note = synthetic construct

SEQ ID NO: 356 - protein, 229 aa, synthetic construct; identical to SEQ ID NO: 352
  REGION 1..229: note = synthetic construct

SEQ ID NO: 357 - protein, 229 aa, synthetic construct
  REGION 1..229: note = synthetic construct
NEREKEIDEGLERVGELISKLKELAREMSEKIEEQNQKLSEIDKKAEEAIKLLEKANASAKKLLEKPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 358 - protein, 229 aa, synthetic construct
  REGION 1..229: note = synthetic construct
NEREKEIEEGLERVGELISELKEMAREMSEKIEEQNKKLDEISKKADEAIKLLEKANKGAEELLKKPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 359 - protein, 229 aa, synthetic construct
  REGION 1..229: note = synthetic construct
NEREKEIDEGLEKIGELISKLKEMAREMSEKIEEQNEKLDEIDKKADEAIKLLEEANKKAEKLLKKKGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 360 - protein, 229 aa, synthetic construct
  REGION 1..229: note = synthetic construct
NEREKEIEEGLERIGELISKLKELAREMSEKIEEQNEKLSEISEKADEAIKLLEKANASAQKLLEKPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 361 - protein, 229 aa, synthetic construct
  REGION 1..229: note = synthetic construct
NPREEEIDKGLEEIGKLISELKELAREMSEKIEEQNEKISEIDEKAKEAIELLKKANEKAKELLEKEGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 362 - protein, 229 aa, synthetic construct
  REGION 1..229: note = synthetic construct
SPREKEIDEGLERVSELVKKLKELAEKMKEMIEEQGRRIERIERKAEEAKERIEKLNEKAEKLLEDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 363 - protein, 229 aa, synthetic construct
  REGION 1..229: note = synthetic construct
SEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 364 - protein, 229 aa, synthetic construct
  REGION 1..229: note = synthetic construct
SEREKEIDEGLEKVSEIVKELKEMAEEMREMIERQGEQIERIEKKAEEAKKKIEEQNERAERLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 365 - protein, 229 aa, synthetic construct
  REGION 1..229: note = synthetic construct
SEREKEIEEGLERVSEIVRRLKELAEEMRRMIEEQGRRIDRIEEKADKAKEEIEKQNEKLEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 366 - protein, 229 aa, synthetic construct
  REGION 1..229: note = synthetic construct
SEREKEIDEGLEKVSEIVKELKELAKEMKEMIEEQGRRIDRIERKAEETKKKIEELNEQAERLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 367 - protein, 229 aa, synthetic construct
  REGION 1..229: note = synthetic construct
SEREEEIDKGLERVSEIVKKLKELAEKMKEEIERQGEQIDRIEKKADETIKEIERLNESADRLLKSPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 368 - protein, 229 aa, synthetic construct; identical to SEQ ID NO: 363
  REGION 1..229: note = synthetic construct

SEQ ID NO: 369 - protein, 229 aa, synthetic construct; identical to SEQ ID NO: 363
  REGION 1..229: note = synthetic construct

SEQ ID NO: 370 - protein, 229 aa, synthetic construct; identical to SEQ ID NO: 363
  REGION 1..229: note = synthetic construct

SEQ ID NO: 371 - protein, 229 aa, synthetic construct; identical to SEQ ID NO: 363
  REGION 1..229: note = synthetic construct

SEQ ID NO: 372 - protein, 229 aa, synthetic construct; identical to SEQ ID NO: 363
  REGION 1..229: note = synthetic construct

SEQ ID NO: 373 - protein, 229 aa, synthetic construct; identical to SEQ ID NO: 363
  REGION 1..229: note = synthetic construct

SEQ ID NO: 374 - protein, 229 aa, synthetic construct; identical to SEQ ID NO: 363
  REGION 1..229: note = synthetic construct

SEQ ID NO: 375 - protein, 229 aa, synthetic construct; identical to SEQ ID NO: 363
  REGION 1..229: note = synthetic construct

SEQ ID NO: 376 - protein, 229 aa, synthetic construct; identical to SEQ ID NO: 363
  REGION 1..229: note = synthetic construct

SEQ ID NO: 377 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKESLANKEERHKQILELEEKIKELYEMFKELSEKIEEQLKKIDRIEEKVSEASRHVSKGVES

SEQ ID NO: 378 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKTRRTLAEIEERHRQILELEEKIEELYEMFKELSEKISEQGQKISRIEDKVSKASEHVSKGVEN

SEQ ID NO: 379 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKSLAEIEKRHEQILQLEKQIEELHEMFKELSEKISKQGQKIDRIEEKVEEAKRHVEKAVKD

SEQ ID NO: 380 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKKVKEELARIEARHQQILALEEKIRELYEMFKELSEKIEEQGKKIDRIEDKVSKASEHVSKGVEN

SEQ ID NO: 381 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKKVKEELKEKEKRHRQIEELLKKIEELHEMFEELSERISEQGQKIDRIDDKVSKASEHVSKGVED

SEQ ID NO: 382 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEEVKKSLAEIEKRHEQILALEKKIEELYEMFKELGEKIEKQLQKISRIEEKVSEASRHVSKGVED

SEQ ID NO: 383 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEIEARHQQIEALLEQIKELYEMFKELSEKIEEQGQKISRIEDKVSKASEHVSKGVEQ

SEQ ID NO: 384 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEKEKRHKQIDELLEKIKELYEMFKEMGEKIEKQGEKIDRIEKKVSEASKHVSKAVED

SEQ ID NO: 385 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEAVKKELAAIEARHKQIDALLEKIKELHEMFEEMSKKIEEQMQKISRIEDKVSEASRHVSKAVSD

SEQ ID NO: 386 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELAEIEKRHKQILELEEKIKELHEMFKELGEKIEKQGQKISRIDDKVSEAKRHVEKGVED

SEQ ID NO: 387 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSKKVKEELKKIEERHKQILELEEKIEELYEMFKELAERIEKQGEKIDRIDEKVSEAKRNVEKAVED

SEQ ID NO: 388 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKKELEEKEKRHKQILELEEKIKELYEMFKELSEKIEEQLQKIDRIDDKVSEASRHVSKGVED

SEQ ID NO: 389 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEEVRRSLEEIERRHRQILELEEKIEELYEMFKEMSEKIEEQGQKISRIEEKVSKASEHVSKAVED

SEQ ID NO: 390 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEEVKKELAEIEARHEQIKELEKQIEELHEMFKELGEKIEKQGEKIDRIDEKVSEASRHVSKAVED

SEQ ID NO: 391 - protein, 229 aa, synthetic construct
  REGION 1..229: note = synthetic construct
DARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKEELAKKEARHEQILELTEKIEELSEMFKELSEKISEQGQKIDRIEDKVSKASEHVSKAVED

SEQ ID NO: 392 - protein, 209 aa, synthetic construct
  REGION 1..209: note = synthetic construct
SELEKKIDELLEEISRLVRELKEIAKELRRLTERQGRQVERIEREVEEAEREIEELNKEAEELLEKEDSEDELEKLKKLLEESKEQLREVERIEREVRRLREEQRRLLELTERAARLAEEALEKMEKMLELQEKILESMKEPDKPYTPEELEKVKERHELIKKLKEEIKELKEMFEELRELVRRQGERLDRIEEKVRRAVEHVKKAEEN

SEQ ID NO: 393 - protein, 209 aa, synthetic construct
  REGION 1..209: note = synthetic construct
SEKEKEIDELLDKVSEIVKELKKLAEELKRRTERQGRQIEEIERKTEEAKRKIEELNKKAEELLKKEDDDSDLEKTKELLKEAKEQLREVKEIKRRVEELKREQEETLKLTKEAAELAEEAKELMEEMLELSEEILEEMLENPKPYTPEELEKVRERHELIKKLLEEIEELEEMFEELERLVEEQGRRLERIEEKVSRAVRHVERAEEN

SEQ ID NO: 394 - protein, 209 aa, synthetic construct
  REGION 1..209: note = synthetic construct
SSKEEEIEELLDEVSEIVRRLKEMAREIREMVERQGRQIERIERKVEEAKRKIEELNKKAEELLEKEDDESELEELEKLVEEAKEQLREVEEINREVEELGREQERLLRKTREAAKLAEKAEELMKKMLELSEEILEEMKEKPKEYTPEELEEVEERHKLIQKLLEEIKELKEMFEELERLVEEQGRRLERIEEKVRRAVEHVKRALEN

SEQ ID NO: 395 - protein, 209 aa, synthetic construct; identical to SEQ ID NO: 392
  REGION 1..209: note = synthetic construct

SEQ ID NO: 396 - protein, 209 aa, synthetic construct; identical to SEQ ID NO: 393
  REGION 1..209: note = synthetic construct

SEQ ID NO: 397 - protein, 229 aa, synthetic construct
  REGION 1..229: note = synthetic construct
SETEKKFNEALEELERLLEEAKKLLEEQRRLLEAQGEVQKEQEKLFDELEEIQEEAEKYQNKLLESKDEEDEMSLLKKALELLEKASKLLEELEALLKKQKELLEKQKELMKELEEVLKKIEEKLKKIKELQEEELKEKKKELAEAEAAEKAGKAAGVSLKEEVEKVKRELAQIEERHQQILELEEKIKELLEMFKELSEKIEEQGQKIDRIEDKVSKAKEHVEKGVED

SEQ ID NO: 398 - protein, 229 aa, synthetic construct
  REGION 1..229: note = synthetic construct
SSLEKKIDENLEKALELLEELKEKLEEMRRLLEESGRLQDELEELMDETQKQQEELEKLLEKLLKMDDSDEQYELLKEALKKQKELKEQLEELEEKLKELRRAHEETRRKMEEAEELLKELEEVMEELKKAQEELLKEKKKKYEEAKKELEEAKKKGEEGKEKLEKVKRELAQIEERHQQILELEEKIKELLEMFKELSEKIEEQGQKIDRIEDKVSKAKEHVEKGVED

SEQ ID NO: 399 - protein, 229 aa, synthetic construct
  REGION 1..229: note = synthetic construct
SEEKKKMEELLDKIKELLEELKKLAEEIKKLLEEQGRQLEKLEEEADKALRQAEEAIRLQEKALELEDDEEIDEALKELEEKQKKLKEQLEKLEEQISELKKLFEEQKKKMEEAEELLKEMLELIKEMKENHEKLLEEAKKRYEEKLKEYEELKKLGILPKEELEKVKRELAQIEERHQQILELEEKIKELLEMFKELSEKIEEQGQKIDRIEDKVSKAKEHVEKGVED

SEQ ID NO: 400 - protein, 229 aa, synthetic construct
  REGION 1..229: note = synthetic construct
EEFEKKENELLEELKKKLEEAKKLLKENRRLLEEQGRQLEEIEEKMEEAEELQEKALEYQEKAEKAGFSDESFEYLKEALKVLEELEEQLEEIEEKLEEQRELLEKQRELLKEAEKKLKEAEEVCKKLKELIEKRKEEAEEKLKKAEEKAKEAAKKGVDLSEELEKVKRELAQIEERHQQILELEEKIKELLEMFKELSEKIEEQGQKIDRIEDKVSKAKEHVEKGVED

SEQ ID NO: 401 - protein, 229 aa, synthetic construct
  REGION 1..229: note = synthetic construct
SEEEEKFNELLEKIEEELEEIKELAEELREKLEELGRLTEKALELADELEKLFEEAEKLLEEALKLGDGEELEEVLKEALEKLKEAKEKLEKLEEELSKLKEAQEEAKELLEELEEELKELEEEIKKLKELSEETLKKAKEKLKEAEKKAEELKKLGIDPTEELEKVKRELAQIEERHQQILELEEKIKELLEMFKELSEKIEEQGQKIDRIEDKVSKAKEHVEKGVED

SEQ ID NO: 402 - protein, 229 aa, synthetic construct
  REGION 1..229: note = synthetic construct
SSLKEKFNELLDELLKELEEAKELLEEIREQLERIGEQLEELEEQFDEILKEQEELEKQQKKLLESPGSEEEEEQLKEIEEKQKKIKEKIEELEEQIEELKEQQEKLKELTKELEEKLKEISETLKELKKVQEELLKEKKKKYEEAKKKYEEKKKKGINGTEELEKVKRELAQIEERHQQILELEEKIKELLEMFKELSEKIEEQGQKIDRIEDKVSKAKEHVEKGVED

SEQ ID NO: 403 - protein, 229 aa, synthetic construct
  REGION 1..229: note = synthetic construct
KESEEKFEEKLKELEKLLEEAKELLEKQREYLEESGRLLDEAEKLMDETERIFEETLKLQDKLLAAKDEEDQMSLLKKALELLEKASKLLDELEATLKELKALLEKQKELMEELEKVLKEIEEKMKEIKKLQEEELKEQKKKLKEAEEKEKEGEKKGVSYKEEVEKVKRELAQIEERHQQILELEEKIKELLEMFKELSEKIEEQGQKIDRIEDKVSKAKEHVEKGVED

SEQ ID NO: 404 - protein, 229 aa, synthetic construct
  REGION 1..229: note = synthetic construct
SKLKEEFEKYLEELLKQLEKLKEKLKELREKLEEQGKQLEKLEEQFDRILEQQEKLLEQQEKLLEDEGSEEEEELLKEIEKQQEKLKEEIEKLEEQIKKLKEQQEELKEISEKAKELLKKTAEILKKLKEVQEKLLEEKKKELEEAEKKYEELKKKGINGTEELEKVKRELAQIEERHQQILELEEKIKELLEMFKELSEKIEEQGQKIDRIEDKVSKAKEHVEKGVED

SEQ ID NO: 405 - protein, 229 aa, synthetic construct
  REGION 1..229: note = synthetic construct
SEREKEFNERLEEMEKLLEKIKKLAEEIRRLLEKQGELLDKLEELADEALRLQEKAIEKSEKILEKGYNEETEEELKELLKLLKELEELLDEAEELIDEIKRLLEEQKKLMEEMEKALKKLEELTKKLKELIEKELEEQRKRLEELEKRKEEYEKLGIDLSEELEKVKRELAQIEERHQQILELEEKIKELLEMFKELSEKIEEQGQKIDRIEDKVSKAKEHVEKGVED

SEQ ID NO: 406 - protein, 229 aa, synthetic construct
  REGION 1..229: note = synthetic construct
SSFEEEINKLLEELKRKLEELKEILEEIRKLLEEQGRQLDEIEEKMDEAEELAEKAEEYLKKAEEAGGGEESYEYLKKALETLKELEEKLDEIEEKLSEQKKLLEETREKLEEAEKKLKEAEKVIKKLKELIEKEKKEKEAELKKAEAAAAEAAKLGIDKSEELEKVKRELAQIEERHQQILELEEKIKELLEMFKELSEKIEEQGQKIDRIEDKVSKAKEHVEKGVED

SEQ ID NO: 407 - protein, 229 aa, synthetic construct
  REGION 1..229: note = synthetic construct
SEEEEKFNELLEKIEEKLEEAKELAEELREELEKIGELTDEAERLADEALKLAEEAEKLLKEALKLGDEDELDKILKEAEKTLEELKKKLEELEEKLKELKEAQEKAKELLKELEETLKELEELIKELKKFSEETLEKAKEKYKKAEEKYKEDLKKGIDNTEEIEKVKRELAQIEERHQQILELEEKIKELLEMFKELSEKIEEQGQKIDRIEDKVSKAKEHVEKGVED

SEQ ID NO: 408 - protein, 229 aa, synthetic construct
  REGION 1..229: note = synthetic construct
ESFEKELEELLEKIQELMEKIKELAEKLREALEESGRLLEEIEEAVDKLEEKFEEIEKLQENAEKYFDTEEAEKYLKEMEEKLKKAKELLDKLEELVSKLKELQEKQRELMEKLEEKLKELLELLKKLKELIEKLKEKKKKELEEAEKKLKEAEEYNEELEKEVEKVKRELAQIEERHQQILELEEKIKELLEMFKELSEKIEEQGQKIDRIEDKVSKAKEHVEKGVED

SEQ ID NO: 409 - protein, 229 aa, synthetic construct
  REGION 1..229: note = synthetic construct
SELEEKFNKYLEELLETLEKLKEALEKIREKLEEQGKQLDKIEEAFDELLKQQEELLKQQEELLADPGSEESEKKLKEIEKQQEKIKEQIEKLEEQIKKLRELQEKQKELTEKAKELLEKLEEILKKLKEVQEKLLEEKKKEYEEAEKEYKEDKKKGINNKEKLEKVKRELAQIEERHQQILELEEKIKELLEMFKELSEKIEEQGQKIDRIEDKVSKAKEHVEKGVED

SEQ ID NO: 410 - protein, 229 aa, synthetic construct; identical to SEQ ID NO: 398
  REGION 1..229: note = synthetic construct

SEQ ID NO: 411 - protein, 229 aa, synthetic construct; identical to SEQ ID NO: 398
  REGION 1..229: note = synthetic construct

SEQ ID NO: 412 - protein, 229 aa, synthetic construct
  REGION 1..229: note = synthetic construct
GELKEKKEKLSKEFEKLLKESKRLAEELKEKLEELGRALDEAEELADEVERQQEELEKLQEEILKSEENEDEKKQLEELEKKLKELEELLKELEEKLKEVEELMKEVEELMEELEKTMEEMEKAIEELEKVYKEELKKTEAKLKATKAEAEAAKAKGEDISDKLEAAEKEYKSVKEELKLVEEIKKKVEEIKEMLEEMKERIEEMEEKVKRIEEKLKRIEESLKRVEEN

SEQ ID NO: 413 - protein, 229 aa, synthetic construct
  REGION 1..229: note = synthetic construct
DELEKKIKELEEKSEEELKEAKELAEELRRLLEELERALDEAERLADEVERKQEELEKLMEEMLKSEDNESDEEDLKKLKEKLEELEKLLEELEERAREVEELMERVEETMEELEEEMEELLETLKKLLEVYEELLKKKKKELEETKKKAEEMKKKGIDISEELEKAKEELESVKKNLELVKKILEEVKEIKEELEEMGEEIERMEEKVDRIEEKLERVEESLERVSKN

SEQ ID NO: 414 - protein, 229 aa, synthetic construct
  REGION 1..229: note = synthetic construct
SEEDKKMEELLEEALKLLEELKELLEKNRELLEELGRQQEELEKLQDEAERLQEELEEAFKKMEENEESEEGKKYLEEAEKLLKELKKLLEEIEKKTKEIEELVKKQEELMKKIKEVMKKLEEKMKELYRISKERLERAKEEAARAEAARAEYEAAGSPEVERAEQVLEEYREAKEFYEKVEELLREVKEIKEEIKEMEERIKEIGERIKRIEEKIERVEKLLERTEKN

SEQ ID NO: 415 - protein, 229 aa, synthetic construct
  REGION 1..229: note = synthetic construct
SEKEKEFNELLEEALRELEKLKELLEENGRLLERTGEQLERMEELMDEAEEKQEELEEAIKKMEKYEDSEEGDEYLEEAEELLEELEELLEEIEAQTEEIEALIKEQEELMKKIKEEMEKLKEAVEKLYEISKEMLEEAKKEYEKAEKAKAEYEAAGKDEVKECEKVKEKYEEAKKRYEQVEKLLKEVEEIKEEIERMGEEIKRQGERIERIEEKIERVEEELERLEEN

SEQ ID NO: 416 - protein, 209 aa, synthetic construct
  REGION 1..209: note = synthetic construct
KPGEEKLNKLLEELLKKLEELKKLAEENRRLLERQGRQLEELERRFEELNRRMEELNEKLEKLLKEEPNEETGEKLEEIKKELEELSRELKELEERVRRQEEEHERQREVVEEIKKELEEAKKYCEELLKTSEEILEEMLENPKPYTPEELEKVRERHELIKKLLEEIEELEEMFEELERLVEEQGRRLERIEEKVSRAVRHVERAEEN

SEQ ID NO: 417 - protein, 209 aa, synthetic construct
  REGION 1..209: note = synthetic construct
EPKEKELEELLEELLRELEEIKKLLEEFRRLQEEIGRQIEEIERQLEELLRRLEELNEKLENLLKREDNENDLEELKELLEEMRELGREMRELERRVEELGRLLEEQRRLVEELKKKLERLLELVKRLLELVEEILEEMLENPKPYTPEELEKVRERHELIKKLLEEIEELEEMFEELERLVEEQGRRLERIEEKVSRAVRHVERAEEN

SEQ ID NO: 418 - protein, 209 aa, synthetic construct
  REGION 1..209: note = synthetic construct
RPEEEKLNELLDELLRLLEEIKKLLEENRALLEEIGRQIDRIEEQLDRLLRELKELNEKLEALLKREDNENDLEELKELLEEIKRLSEEMKELEREVERLGELLEEQRRKVEELKRKLEELLELTEEALELVEEILEEMLENPKPYTPEELEKVRERHELIKKLLEEIEELEEMFEELERLVEEQGRRLERIEEKVSRAVRHVERAEEN

SEQ ID NO: 419 - protein, 189 aa, synthetic construct
  REGION 1..189: note = synthetic construct
SLEEILEKLKEIAELLEEVEELTEELKEETERAGRELEELERRLEELVRRAEELNRKLEKILEEEDSDDILERLKEARRELRELRERLEEVEREIERLIREAEEQSELLEELERELEEIKELLKELLEKEEELSEEELELIKKLLEEIEELEEMFEELERLVEEQGRRLERIEEKVSRAVRHVERAEEN

SEQ ID NO: 420 - protein, 335 aa, synthetic construct
  REGION 1..335: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSDARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 421 - protein, 347 aa, synthetic construct
  REGION 1..347: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSGGSGGSGGSGGSDARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 422 - protein, 335 aa, synthetic construct
  REGION 1..335: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSSEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 423 - protein, 347 aa, synthetic construct
  REGION 1..347: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSGGSGGSGGSGGSSEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 424 - protein, 329 aa, synthetic construct
  REGION 1..329: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSSEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVED

SEQ ID NO: 425 - protein, 309 aa, synthetic construct
  REGION 1..309: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSSEKEKEIDELLDKVSEIVKELKKLAEELKRRTERQGRQIEEIERKTEEAKRKIEELNKKAEELLKKEDDDSDLEKTKELLKEAKEQLREVKEIKRRVEELKREQEETLKLTKEAAELAEEAKELMEEMLELSEEILEEMLENPKPYTPEELEKVRERHELIKKLLEEIEELEEMFEELERLVEEQGRRLERIEEKVSRAVRHVERAEEN

SEQ ID NO: 426 - protein, 15 aa, synthetic construct
  REGION 1..15: note = synthetic construct
TKKAVKYQSRRRRRR

SEQ ID NO: 427 - protein, 15 aa, synthetic construct
  REGION 1..15: note = synthetic construct
TKKAVKYQSKKKKKK

SEQ ID NO: 428 - protein, 15 aa, synthetic construct
  REGION 1..15: note = synthetic construct
LKEAVEYDEKARRKK

SEQ ID NO: 429 - protein, 15 aa, synthetic construct
  REGION 1..15: note = synthetic construct
LKKAVEYDEKARRKK

SEQ ID NO: 430 - protein, 15 aa, synthetic construct
  REGION 1..15: note = synthetic construct
LKEAVEYEEKARRKK

SEQ ID NO: 431 - protein, 15 aa, synthetic construct
  REGION 1..15: note = synthetic construct
LKEAVKYREESEKME

SEQ ID NO: 432 - protein, 15 aa, synthetic construct
  REGION 1..15: note = synthetic construct
LKEAVKYNEEGKKME

SEQ ID NO: 433 - protein, 15 aa, synthetic construct
  REGION 1..15: note = synthetic construct
LKEAVEYREKSEKME

SEQ ID NO: 434 - protein, 15 aa, synthetic construct
  REGION 1..15: note = synthetic construct
LKEAVKYKEESEKME

SEQ ID NO: 435 - protein, 15 aa, synthetic construct
  REGION 1..15: note = synthetic construct
LKKAVEYKEKSEKKE

SEQ ID NO: 436 - protein, 15 aa, synthetic construct
  REGION 1..15: note = synthetic construct
TKKAVKYQSESEKME

SEQ ID NO: 437 - protein, 15 aa, synthetic construct
  REGION 1..15: note = synthetic construct
TKKAVKYQSEAEKME

SEQ ID NO: 438 - protein, 15 aa, synthetic construct
  REGION 1..15: note = synthetic construct
TKKAVKYQSEAEKKE

SEQ ID NO: 439 - protein, 15 aa, synthetic construct
  REGION 1..15: note = synthetic construct
IGEAVKYLEKSKELE

SEQ ID NO: 440 - protein, 15 aa, synthetic construct
  REGION 1..15: note = synthetic construct
LGEAVEYLEKSKKLE

SEQ ID NO: 441 - protein, 15 aa, synthetic construct
  REGION 1..15: note = synthetic construct
LKEAKEYRKKNEELE

SEQ ID NO: 442 - protein, 15 aa, synthetic construct
  REGION 1..15: note = synthetic construct
LKEIKELAKKREEKG

SEQ ID NO: 443 - protein, 15 aa, synthetic construct
  REGION 1..15: note = synthetic construct
LEEIERLFEERKEKG

SEQ ID NO: 444 - protein, 15 aa, synthetic construct
  REGION 1..15: note = synthetic construct
LKEIKELRDKIEKNG

SEQ ID NO: 445 - protein, 15 aa, synthetic construct
  REGION 1..15: note = synthetic construct
LEEIKKLREKIKENG

SEQ ID NO: 446 - protein, 247 aa, synthetic construct
  REGION 1..247: note = synthetic construct
KPGEEKLNKLLEELLKKLEELKKLAEENRRLLERQGRQLEELERRFEELNRRMEELNEKLEKLLKEEPNEETGEKLEEIKKELEELSRELKELEERVRRQEEEHERQREVVEEIKKELEEAKKYCEELLKTSEEILEEMLENPKPYTPEELEKVRERHELIKKLLEEIEELEEMFEELERLVEEQGRRLERIEEKVSRAVRHVERAEENLGEAVEYLEKSKKLEIMIIICCVILGIVIASTVGGIFA

SEQ ID NO: 447 - protein, 247 aa, synthetic construct
  REGION 1..247: note = synthetic construct
EPKEKELEELLEELLRELEEIKKLLEEFRRLQEEIGRQIEEIERQLEELLRRLEELNEKLENLLKREDNENDLEELKELLEEMRELGREMRELERRVEELGRLLEEQRRLVEELKKKLERLLELVKRLLELVEEILEEMLENPKPYTPEELEKVRERHELIKKLLEEIEELEEMFEELERLVEEQGRRLERIEEKVSRAVRHVERAEENLGEAVEYLEKSKKLEIMIIICCVILGIVIASTVGGIFA

SEQ ID NO: 448 - protein, 247 aa, synthetic construct
  REGION 1..247: note = synthetic construct
RPEEEKLNELLDELLRLLEEIKKLLEENRALLEEIGRQIDRIEEQLDRLLRELKELNEKLEALLKREDNENDLEELKELLEEIKRLSEEMKELEREVERLGELLEEQRRKVEELKRKLEELLELTEEALELVEEILEEMLENPKPYTPEELEKVRERHELIKKLLEEIEELEEMFEELERLVEEQGRRLERIEEKVSRAVRHVERAEENLGEAVEYLEKSKKLEIMIIICCVILGIVIASTVGGIFA

SEQ ID NO: 449 - protein, 227 aa, synthetic construct
  REGION 1..227: note = synthetic construct
SLEEILEKLKEIAELLEEVEELTEELKEETERAGRELEELERRLEELVRRAEELNRKLEKILEEEDSDDILERLKEARRELRELRERLEEVEREIERLIREAEEQSELLEELERELEEIKELLKELLEKEEELSEEELELIKKLLEEIEELEEMFEELERLVEEQGRRLERIEEKVSRAVRHVERAEENLGEAVEYLEKSKKLEIMIIICCVILGIVIASTVGGILA

SEQ ID NO: 450 - protein, 373 aa, synthetic construct
  REGION 1..373: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSDARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 451 - protein, 385 aa, synthetic construct
  REGION 1..385: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSGGSGGSGGSGGSDARENEMDENLEQVSGIIGNLRHMALDMGNEIDTQNRQIDRIMEKADSAKTRIDEANQRATKMLGSGGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 452 - protein, 373 aa, synthetic construct
  REGION 1..373: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSSEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 453 - protein, 385 aa, synthetic construct
  REGION 1..385: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSGGSGGSGGSGGSGGSGGSSEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 454 - protein, 367 aa, synthetic construct
  REGION 1..367: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSSEREKEIDEGLDRVSEIVKELKKMAEEMRRMIEEQGRRIERIEEKAEEAKEKIEEANERAEKLLKDPGGSGGSEELKKLEKEGEKLKELVEELDREIKELKEGMERLREMFEEAAKLSEEALEIMRRTRKLSEEELEEAKQKAKEQEKEKALKEKGGSGGSGGSEKVKRELAQIEERHQQILELEEKIKELLEMFKELSEKIEEQGQKIDRIEDKVSKAKEHVEKGVEDTKKAVKYQSKARRKKIMIIICCVILGIVIASTVGGIFA

SEQ ID NO: 455 - protein, 347 aa, synthetic construct
  REGION 1..347: note = synthetic construct
VAILWHEMWHEGLEEASRLYFGERNVKGMFEVLEPLHAMMERGPQTLKETSFNQAYGRDLMEAQEWCRKYMKSGNVKDLTQAWDLYYHVFRRISGGSGGSSEKEKEIDELLDKVSEIVKELKKLAEELKRRTERQGRQIEEIERKTEEAKRKIEELNKKAEELLKKEDDDSDLEKTKELLKEAKEQLREVKEIKRRVEELKREQEETLKLTKEAAELAEEAKELMEEMLELSEEILEEMLENPKPYTPEELEKVRERHELIKKLLEEIEELEEMFEELERLVEEQGRRLERIEEKVSRAVRHVERAEENLGEAVEYLEKSKKLEIMIIICCVILGIVIASTVGGIFA

SEQ ID NO: 456 - protein, 12 aa, synthetic construct
  REGION 1..12: note = synthetic construct
KAKRKYWWKNSK

(19) United States
(12) Patent Application Publication
(10) Pub. No.: US 2025/0092424 A1
(43) Pub. Date: Mar. 20, 2025

(54) Membrane fusion proteins

(71) Applicant: University of Washington, Seattle, WA (US)

(72) Inventors: Neil P. KING, Seattle, WA (US); Masaharu SOMIYA, Seattle, WA (US); Sydney FUNK, Seattle, WA (US)

(21) Appl. No.: 18/884,464

(22) Filed: Sep. 13, 2024

Related U.S. Application Data
(60) Provisional application No. 63/582,937, filed on Sep. 15, 2023.

(51) Int. Cl. C12N 15/88; C07K 14/705; C12N 15/85

(52) U.S. Cl. CPC C12N 15/88; C07K 14/70571; C12N 15/85; C12N 2800/107

(57) ABSTRACT

Nucleic acids and expression vectors, and host cells or vesicles containing them, are provided that encode polypeptides and fusion proteins capable of forming hetero-oligomeric complexes capable of inducing membrane or vesicle fusion, and their use as delivery vehicles.

### SEQUENCE LISTING STATEMENT

A computer readable form of the Sequence Listing is filed with this application by electronic submission and is incorporated into this application by reference in its entirety. The Sequence Listing is contained in the file created on Sep. 10, 2024 having the file name “23-1345-US.xml” and is 635,732 bytes in size.

### BACKGROUND

Membrane fusion is a process of merging of two membranes. In a biological system, membrane fusion proteins catalyze this process by pulling two lipid bilayer membranes into close proximity and forcing them to merge into a single membrane. Enveloped viruses and some viral vectors such as lentiviral or retroviral vectors have membrane fusion proteins on their surface to efficiently fuse the viral membrane and host cell membrane to deliver their genetic material into cells. Therefore, membrane fusion proteins are potentially useful for intracellular drug delivery applications. However, natural membrane fusion proteins including viral envelope glycoproteins or SNARE proteins are hard to engineer and sometimes immunogenic or toxic for biological applications. Newly designed membrane fusion proteins might be advantageous over existing membrane fusion proteins since they could be modular and easier to engineer.

### SUMMARY

In a first aspect, the disclosure provides nucleic acids encoding a polypeptide comprising the formula X1-X2-X3, wherein

X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of the bold font residues in SEQ ID NO: 149-208;

X2 comprises a juxtamembrane domain (JMD), wherein X2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:500-505; and

X3 comprises a transmembrane domain (TMD).

In one embodiment, X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:156. In another embodiment, 1, 2, 3, 4, 5, 6, 7, 8, 9, 10, 11, 12, or all 13 of L8, L12, V15, V18, 121, M22, L28, V29, G33, 136, L39, L46, L53 are conserved (i.e., identical) in the polypeptide relative to SEQ ID NO:156. In a further embodiment, X2 comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:209-222 and 456. In one embodiment, X2 comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO: 213 and 214. In another embodiment, X3 comprises an amino acid at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: SEQ ID NO:223-234. In a further embodiment, the nucleic acid encodes a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-37, 147, and 236-289. In one embodiment, the nucleic acid encodes a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8. In another embodiment, 1, 2, 3, 4, 5, 6, 7, 8, 9, 10, 11, 12, or all 13 of L8, L12, V15, V18, 121, M22, L28, V29, G33, 136, L39, L46, L53 are conserved (i.e., identical) in the polypeptide relative to SEQ ID NO: 8. In other embodiments, the nucleic acids encode a fusion protein that further comprises an amino acid sequence at least 90%, 91%, 92%, 93%, 94%, 95%, 96%, 97%, 98%, 99%, or 100% identical to the amino acid sequence of SEQ ID NO: 290 or 291; optionally wherein the encoded polypeptide and the polypeptide domain are connected by an amino acid linker. In certain embodiments, the nucleic acid encodes a fusion protein comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:147 and 244-258.

In a second aspect, the disclosure provides nucleic acids encoding a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:310-316, wherein X1 is an amino acid linker. In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 314-316, wherein X1 is an amino acid linker. In another embodiment, the nucleic acid encodes a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:38-44.

In a third aspect, the disclosure provides nucleic acids encoding a polypeptide comprising the formula X1-X2-X3, wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 317-330;
X2 comprises a juxtamembrane domain (JMD); and
X3 comprises a transmembrane domain (TMD).

In one embodiment, X2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:331-332. In another embodiment, X3 comprises an amino acid at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 223-234. In a further embodiment, the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:45-63.

In a fourth aspect, the disclosure provides nucleic acids encoding a polypeptide comprising the formula X1-X2-X3, wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of the first set bold font residues in SEQ ID NO: 333-425, wherein the non-highlighted residues are amino acid linkers that may be substituted with any other amino acid linker;
X2 comprises a juxtamembrane domain (JMD); and
X3 comprises a transmembrane domain (TMD).

In one embodiment, X2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 331-332 and 426-445. In another embodiment, X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:223-234. In a further embodiment, the polypeptide formula comprises B1-B2-X1-X2-X3, wherein
B1 comprises the amino acid sequence at least 90%, 91%, 92%, 93%, 94%, 95%, 96%, 97%, 98%, 99%, or 100% of SEQ ID NO:290 or 291; and
B2 comprises an optional amino acid linker, which may be present or absent.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 64-146, 148, and 446-455.

In all aspects, the nucleic acids may encode a polypeptide that further comprises a signal peptide at its amino-terminus, including but not limited to a signal peptide that comprises the amino acid sequence selected from the group consisting of SEQ ID NO: 292-309. In all aspects, the nucleic acids may comprise an expression vector comprising the nucleic acid operatively linked to a control sequence, such as a promoter.

The disclosure also provides polypeptides or fusion proteins encoded by the nucleic acid of any aspect or embodiment of the disclosure, and host cell comprising the nucleic acid, expression vector, polypeptide, and/or fusion protein of any aspect or embodiment of the disclosure. In one embodiment, the host cell comprises a membrane fusion protein complex anchored in a lipid bilayer membrane of the cell, wherein the membrane fusion protein complex comprises the following components:
(a) a polypeptide encoded by the nucleic acid of any embodiment of the first aspect of the disclosure; and
(b) a polypeptide encoded by the nucleic acid of any embodiment of the second aspect of the disclosure; and
(c) a polypeptide encoded by the nucleic acid of any embodiment of the third aspect of the disclosure;
wherein components (a)-(c) form a hetero-oligomeric complex anchored in a lipid bilayer membrane of the cell, wherein the hetero-oligomeric complex is capable of inducing membrane fusion.

In another embodiment, the host cell comprises a membrane fusion protein complex anchored in a lipid bilayer membrane of the cell, wherein the membrane fusion protein complex comprises the following components:
(a) a polypeptide encoded by the nucleic acid of any embodiment of the first aspect of the disclosure; and
(b) a polypeptide encoded by the nucleic acid of any embodiment of the fourth aspect of the disclosure;
wherein components (a)-(b) form a hetero-oligomeric complex anchored in a lipid bilayer membrane of the cell, wherein the hetero-oligomeric complex is capable of inducing membrane fusion.

The disclosure also provides vesicles, comprising one or more polypeptide or fusion protein of any aspect or embodiment herein incorporated into the lipid envelope of the vesicle. In various embodiments, the vesicle comprises a liposome, a lipid nanoparticle, a viral vector, or an enveloped particle that may optionally comprise any suitable cargo, including but not limited to a protein or nucleic acid cargo. In other embodiments, one or more polypeptide or fusion protein of any aspect or embodiment disclosed here are anchored on a surface of the liposome, the lipid nanoparticle, the viral vector, or the enveloped particle.

In one embodiment, the host cell or vesicle of any embodiment herein, further comprises a therapeutic or diagnostic moiety loaded in the host cell or vesicle.

The disclosure also provides kits. In one embodiment, the kit comprises
(a) a first host cell or vesicle comprising the nucleic acid of any embodiment of the first aspect of the disclosure; and
(b) a second host cell or vesicle comprising the nucleic acid of any embodiment of the second aspect of the disclosure, and the nucleic acid of any embodiment of the third aspect of the disclosure. In one embodiment, the first host cell or vesicle comprises a polypeptide encoded by the nucleic acid of any embodiment of the first aspect of the disclosure anchored in a lipid bilayer membrane of the cell or vesicle. In another embodiment, the second host cell or vesicle comprises a polypeptide encoded by the nucleic acid of any embodiment of the second aspect of the disclosure anchored in a lipid bilayer membrane of the cell or vesicle.

In another embodiment, the kit comprises
(a) a first host cell or vesicle comprising the nucleic acid of any embodiment of the first aspect of the disclosure; and
(b) a second host cell or vesicle comprising polypeptide encoded by the nucleic acid of any embodiment of the fourth aspect of the disclosure. In one embodiment, the first host cell or vesicle comprises a polypeptide encoded by the nucleic acid of any embodiment of the first aspect of the disclosure anchored in a lipid bilayer membrane of the cell or vesicle. In another embodiment, the second host cell or vesicle comprises a polypeptide encoded by the nucleic acid of any embodiment of the forth aspect of the disclosure anchored in a lipid bilayer membrane of the cell or vesicle.

The disclosure also provides methods for inducing membrane fusion. In one embodiment, the method comprises mixing
(a) a first host cell or vesicle comprising a polypeptide encoded by the nucleic acid of any embodiment of the first aspect of the disclosure anchored in a lipid bilayer membrane of the cell; and
(b) a second host cell or vesicle comprising a polypeptide encoded by the nucleic acid of any embodiment of the third aspect of the disclosure anchored in a lipid bilayer membrane of the cell; wherein the polypeptide encoded by the nucleic acid of any embodiment of the third aspect of the disclosure is non-covalently bound to a polypeptide encoded by the nucleic acid of any embodiment of the second aspect of the disclosure;
under conditions to promote fusion of the first host cell or vesicle and the second host cell or vesicle.

In another embodiment, the method comprises mixing:
(a) a first host cell or vesicle comprising a polypeptide encoded by the nucleic acid of any embodiment of the first aspect of the disclosure anchored in a lipid bilayer membrane of the cell; and
(b) a second host cell or vesicle comprising a polypeptide encoded by the nucleic acid of any embodiment of the fourth aspect of the disclosure;
under conditions to promote fusion of the first host cell or vesicle and the second host cell or vesicle.

### DESCRIPTION OF THE FIGURES

FIG. 1. Syntaxin exists in a closed conformation that opens to initiate core-complex assembly (nucleation). ‘Zippering’ of the four-helix bundle towards the carboxyl terminus brings the synaptic vesicle and plasma membranes towards each other, which might lead to membrane fusion. After fusion, N-ethylmaleimide-sensitive fusion protein (NSF) and soluble NSF-attachment proteins (SNAPs) disassemble the cis-core complexes that remain on the same membrane to recycle them for another round of fusion. SNAP25, synaptosomal-associated protein of 25 kDa; SNARE, SNAP receptor. Figure and caption from Rizo & Sudhof, Nat Rev Neurosci 3, 641-653 (2002).

FIG. 2. Synthetic SNARE engineering trajectory.

FIG. 3. The structure of the neuronal SNARE complex. Predicted three-dimensional structure of human neuronal SNARE complex. The model was generated by the protein structure prediction method ColabFold³. VAMP2, SNAP25, and Syntaxin 1A (N001, N002, and N003). Transmembrane domains at the C terminus are on the right side of the structure.

FIG. 4. Fusion activity of MPNN-redesigned v-SP (SEQ ID: 1-18). The fusion activity of design #8 was evaluated in a cell-cell fusion assay. Design #8 was expressed in HEK293T cells (v-cells) and native t-SNAREs (SNAP25 and Syn1A) were expressed in another HEK293T population (t-cells). These proteins were expressed on the surface of HEK293T cells as flipped SNARE³ (thus “f” before the name of native SNARE proteins denotes “flipped”). The v- and t-cells were mixed together and after overnight incubation, cell-cell fusion was quantitatively assessed by reporter gene expression (RLU on the y-axis represents the relative luminescence unit that is calculated by reporter luciferase activity). As controls, fVAMP2-WT (native SNARE protein) and no SNARE expression were used.

FIG. 5. Fusion activity of single chain-t-SP (SEQ ID: 64-122). Design #8 (v-SP) and design #94 (sc-t-SP) were expressed in v- and t-cells respectively. The fusion activity of proteins was quantitatively measured by cell-cell fusion assay based on reporter gene expression. As controls, fVAMP2-WT, fSyn1A, and fSNAP25 (native SNARE proteins) and no SNARE expression were used.

FIG. 6. Fusion activity of shorter versions of sc-t-SP (SEQ ID: 123-127). Design #124 (sc-t-SP) has a 20-aa shorter length compared to parental sc-t-SPs (design #94), and has slightly weaker fusion activity compared to longer versions of sc-t-SPs, but the fusion activity was still significantly stronger than the native SNARE complex (fVAMP2-WT, fSyn1A, and fSNAP25). Design #8 was used as v-SP with sc-t-SPs. Fusion activity was measured by cell-cell fusion assay.

FIG. 7. Fusion activity of partially diffused sc-t-SP (SEQ ID: 128-146). The sc-t-SP design #143 has a partially diffused backbone compared to the parental sc-t-SP and has comparable fusion activity to design #94. Design #8 was used as v-SP with sc-t-SPs. Fusion activity was measured by cell-cell fusion assay.

FIG. 8. Inducible fusion activity of newly fusion proteins (SEQ ID: 147-148). The N termini of v-SP and sc-t-SP were fused with FKBP (design #147) and FRB (design #148), respectively, and expressed in HEK293T cells. Rapamycin was added to the mixture of v- and t-cells at the indicated concentrations, which induced heterodimerization of v- and t-SPs and cell-cell fusion in a dose-dependent manner. Fusion activity was measured by cell-cell fusion assay.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Any N-terminal amino acids are optional, and may be deleted.

The polypeptides of all aspects and embodiments of the disclosure are able, for example, to form a heterooligomer with corresponding other protein(s) of the disclosure on a lipid bilayer membrane (as discussed in more detail below), followed by induction of merger of two membranes into a single continuous membrane.

VAMP2-Redesign (v-SNARE-Like Proteins (v-SPs))

In a first aspect, the disclosure provides nucleic acids encoding a v-SNARE-like protein (v-SP) polypeptide comprising the formula X1-X2-X3, wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of the bold font residues in SEQ ID NO:149-208;
X2 comprises a juxtamembrane domain (JMD), wherein X2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:500-505; and
X3 comprises a transmembrane domain (TMD).

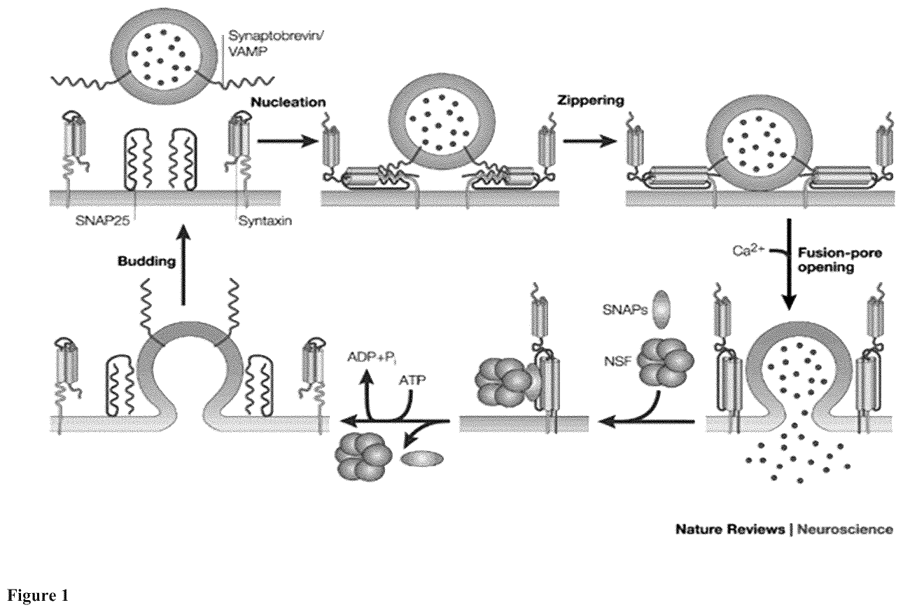

As described in the examples, the disclosure provides a series of membrane fusion proteins that can induce cell-cell fusion when expressed on the surface of mammalian cells, or liposome fusion when displayed on the surface of liposomes. The designed proteins are based on the human neuronal SNARE complex (which is composed of three proteins, VAMP2, Syntaxin 1A or Syn1A, and SNAP25), which has a parallel four-helical bundle structure and transmembrane domains at the C-terminus of VAMP2 and Syn1A (see FIG. 1). VAMP2 is called v-SNARE, and Syn1A and SNAP25 are called t-SNARE since they exist on the vesicle (v-) or target (t-) membrane inside cells. The four alpha helices of the SNARE complex are composed of one helix from each of VAMP2 and Syn1A and two helices from SNAP25. The nucleic acids of this aspect encode the helix (X1 domain) and membrane domains (X2 and X3) of redesigned VAMP2 (hereafter v-SNARE-like proteins or v-SPs).

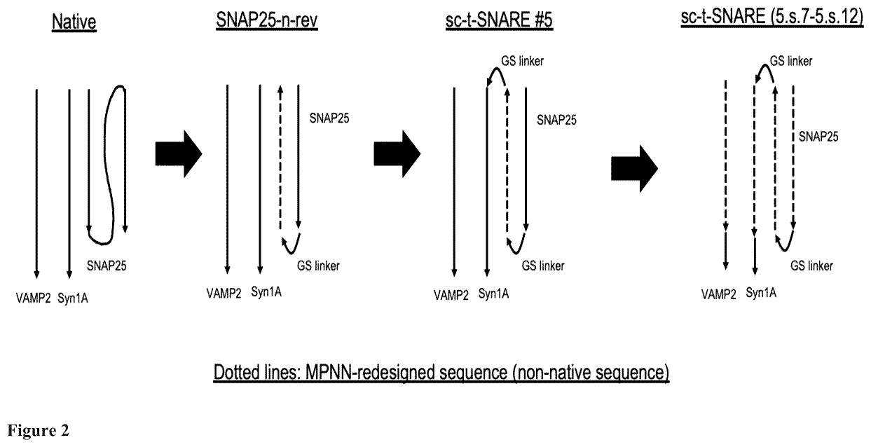

As further described in the examples, new sequences were generated that are believed to fold into the four-helix bundle structure like the parental SNARE complex, followed by engineering SNAP25 so that one of the two coiled-coil domains of SNAP25 is an anti-parallel coiled-coil (FIG. 2, first modification). By combining engineered SNAP25 which has one anti-parallel coiled-coil domain and Syn1A into a single protein, a two-component fusion machinery was generated (one v-SP and one t-SNARE, rather than three components of the original neuronal SNARE; FIG. 2, second modification). Based on the single chain t-SNARE (sc-t-SNARE) backbone, dozens of further sequences that are capable of inducing membrane fusion in a mammalian cell-cell fusion assay (5.s.7-5.s.12 in FIG. 2, third modification) were generated, including those that showed a 10-fold increased fusion efficiency compared to the parental neuronal SNARE complex. These sc-t-SNAREs are designated as single-chain t-SNARE-like proteins or sc-t-SPs.

The amino acid sequences of the encoded X1 domains (SEQ ID NO:149-208) are provided in Tables 1 and 2 below.

TABLE 1
>SEQ ID NO: 149
SSSSSEKLRETQAQVDEVVDIMRVNVDKVLERDQKLSELDDRADALQAGASQFETSAA
> SEQ ID NO: 150
NLASNRRLQQTSEEVREVNDIMRVNVDKVLERDQKLSELDDRADALQAGASQFETSAA
> SEQ ID NO: 151
NLASNRRLQQTQAQVDEVVDVMRDNRNLVDERDQKLSELDDRADALQAGASQFETSAA
> SEQ ID NO: 152
NLASNRRLQQTQAQVDEVVDIMRVNVDKVLRQGEQIDRLEDRADALQAGASQFETSAA
> SEQ ID NO: 153
NLASNRRLQQTQAQVDEVVDIMRVNVDKVLERDQKLSELDERADELEKSASQFETSAA
> SEQ ID NO: 154
NLASNRRLQQTQAQVDEVVDIMRVNVDKVLERDQKLSELDDRADALQAGAERLEENAT
> SEQ ID NO: 155
NLASNRRLQQTQAQVDEVVDIMRVNVDKVLERDQKLSELDDRADALQAGASQFETSAA
> SEQ ID NO: 156
SSSSNEKLRETLREVEDVKNIMEDNRRLVERQGRQIDRLEEKADDLERSAERLSDNSE
> SEQ ID NO: 157
SSSDSSKLRETEEETRDVIDIMRDNRRSVEERGRQIDRLEERADDLEDSAERLEENAK
> SEQ ID NO: 158
SGSSNERLREVSKEAREVREMAMDVKEKIEEQGRKIEELEEKAESLKDSAERFDENAK
> SEQ ID NO: 159
SGTTNEKLRKVSSEADEVKEMGMDVKEKVEEQGRKIEELEEKAEDLKDSAERFDENAK
> SEQ ID NO: 160
SGSSSEKLRQISSEAEEVKEMGMDILKKIEEQGEKIERLEEKAESLKDSAERFADNAK
> SEQ ID NO: 161
DGTSNERLRETSKEAREVRDMAMDNMKKVEEQGEKIEELEEKAEELKDSAERLDDNAK
> SEQ ID NO: 162
DGTSNEKLRETSEQAREVRDMALDNKEKIEEQGEKIDRLEEKAESLKDSAERFAENAK
> SEQ ID NO: 163
SEEMSKKLEETSKEVDEVLEIMEEIREMLEEQGRRIDRLEKKAEELEEGAEKFEELSE
> SEQ ID NO: 164
SEERKEKLEETLKEVDEVLEIMKENKEMLEEQGERLERLEEKAEELEEGAEKFEELAE
> SEQ ID NO: 165
SKERSEKLKETMEEVEEVLEIMKEIRRMMEEQGERIDRLEEKAEELEEGAEKFEELAE

TABLE 2

| # | amino acid sequence |
|---|---|
| N8 | SSSSNEKLRETSREVEEVNDIMRDNRNLVDRQGEQIDRLEERADELKDSAERLSENSK |
| | SEQ ID NO: 166 |
| N13 | SSSSNEKLRETQAQVDEVVDIMRVNVDKVLERDQKLSELDDRADALQAGASQFETSAA |
| | SEQ ID NO: 167 |
| N14 | NLASNRRLQQTLREVEDVKNIMRVNVDKVLERDQKLSELDDRADALQAGASQFETSAA |
| | SEQ ID NO: 168 |
| N15 | NLASNRRLQQTQAQVDEVVDIMEDNRRLVEERDQKLSELDDRADALQAGASQFETSAA |
| | SEQ ID NO: 169 |
| N16 | NLASNRRLQQTQAQVDEVVDIMRVNVDKVLRQGRQIDRLEDRADALQAGASQFETSAA |
| | SEQ ID NO: 170 |
| N17 | NLASNRRLQQTQAQVDEVVDIMRVNVDKVLERDQKLSELDEKADDLERSASQFETSAA |
| | SEQ ID NO: 171 |
| N18 | NLASNRRLQQTQAQVDEVVDIMRVNVDKVLERDQKLSELDDRADALQAGAERLSDNSE |
| | SEQ ID NO: 172 |
| N21 | NLASNRRLQQTLREVEDVKNIMEDNRRLVERQGRQIDRLEEKADDLERSAERLSDNSE |
| | SEQ ID NO: 173 |
| N22 | SSSSNEKLRETQAQVDEVVDIMEDNRRLVERQGRQIDRLEEKADDLERSAERLSDNSE |
| | SEQ ID NO: 174 |
| N23 | SSSSNEKLRETLREVEDVKNIMRVNVDKVLRQGRQIDRLEEKADDLERSAERLSDNSE |
| | SEQ ID NO: 175 |
| N24 | SSSSNEKLRETLREVEDVKNIMEDNRRLVEERDQKLSELDEKADDLERSAERLSDNSE |
| | SEQ ID NO: 176 |
| N25 | SSSSNEKLRETLREVEDVKNIMEDNRRLVERQGRQIDRLEDRADALQAGAERLSDNSE |
| | SEQ ID NO: 177 |
| N26 | SSSSNEKLRETLREVEDVKNIMEDNRRLVERQGRQIDRLEEKADDLERSASQFETSAA |
| | SEQ ID NO: 178 |
| N34 | SEEEEKKKEELKKKLKEALEEAKKAKELAKKALELAERQGRQIDRLEEKADDLERSAE |
| | RLSDNSE |
| | SEQ ID NO: 179 |
| N35 | EEEKEKKKEELKEKAKKALEEAKKTKELAKEALELAERQGRQIDRLEEKADDLERSAE |
| | RLSDNSE |
| | SEQ ID NO: 180 |
| N36 | SLEAEKKEKEEKEKKKKILELLKELLEETEELKEEAEEIKREVERQGRQIDRLEEKAD |
| | DLERSAERLSDNSE |
| | SEQ ID NO: 181 |
| N37 | ELEEELKKKEEEEKRKEILELLKELLEETEELKEEAEEIKEEVERQGRQIDRLEEKAD |
| | DLERSAERLSDNSE |
| | SEQ ID NO: 182 |
| N38 | SSSSNEKARETLREVEDVKNIMEDNRRLVERQGRQIDRLEEKADDLERSAERLSDNSE |
| | SEQ ID NO: 183 |
| N39 | SSSSNEKLRETAREVEDVKNIMEDNRRLVERQGRQIDRLEEKADDLERSAERLSDNSE |
| | SEQ ID NO: 184 |
| N40 | SSSSNEKLRETLREAEDVKNIMEDNRRLVERQGRQIDRLEEKADDLERSAERLSDNSE |
| | SEQ ID NO: 185 |
| N41 | SSSSNEKLRETLREVEDAKNIMEDNRRLVERQGRQIDRLEEKADDLERSAERLSDNSE |
| | SEQ ID NO: 186 |
| N42 | SSSSNEKLRETLREVEDVKNAMEDNRRLVERQGRQIDRLEEKADDLERSAERLSDNSE |
| | SEQ ID NO: 187 |
| N43 | SSSSNEKLRETLREVEDVKNIAEDNRRLVERQGRQIDRLEEKADDLERSAERLSDNSE |
| | SEQ ID NO: 188 |
| N44 | SSSSNEKLRETLREVEDVKNIMEDNRRAVERQGRQIDRLEEKADDLERSAERLSDNSE |
| | SEQ ID NO: 189 |
| N45 | SSSSNEKLRETLREVEDVKNIMEDNRRLAERQGRQIDRLEEKADDLERSAERLSDNSE |
| | SEQ ID NO: 190 |
| N46 | SSSSNEKLRETLREVEDVKNIMEDNRRLVERQARQIDRLEEKADDLERSAERLSDNSE |
| | SEQ ID NO: 191 |
| N47 | SSSSNEKLRETLREVEDVKNIMEDNRRLVERQGRQADRLEEKADDLERSAERLSDNSE |
| | SEQ ID NO: 192 |
| N48 | SSSSNEKLRETLREVEDVKNIMEDNRRLVERQGRQIDRAEEKADDLERSAERLSDNSE |
| | SEQ ID NO: 193 |
| N49 | SSSSNEKLRETLREVEDVKNIMEDNRRLVERQGRQIDRLEEKADDAERSAERLSDNSE |
| | SEQ ID NO: 194 |
| N50 | SSSSNEKLRETLREVEDVKNIMEDNRRLVERQGRQIDRLEEKADDLERSAERASDNSE |
| | SEQ ID NO: 195 |
| N52 | SSSSNEKARETAREAEDVKNIMEDNRRLVERQGRQIDRLEEKADDLERSAERLSDNSE |
| | SEQ ID NO: 196 |
| N53 | SSSSNEKARETAREVEDVKNIMEDNRRLVERQGRQIDRLEEKADDLERSAERLSDNSE |
| | SEQ ID NO: 197 |
| N54 | SSSSNEKARETAREAEDAKNIMEDNRRLVERQGRQIDRLEEKADDLERSAERLSDNSE |
| | SEQ ID NO: 198 |
| N55 | SSSSNEKARETAREAEDAKNAMEDNRRLVERQGRQIDRLEEKADDLERSAERLSDNSE |
| | SEQ ID NO: 199 |
| N56 | SSSSNEKARETAREAEDAKNAAEDNRRLVERQGRQIDRLEEKADDLERSAERLSDNSE |
| | SEQ ID NO: 200 |
| N57 | SSSSNEKARETLREVEDVKNIAEDNRRLVERQGRQIDRAEEKADDLERSAERLSDNSE |
| | SEQ ID NO: 201 |
| N58 | SSSSNEKLKETLKEVEDVKNIMEDNKKLVEKQGKQIDKLEEKADDLEKSAEKLSDNSE |
| | SEQ ID NO: 202 |
| N59 | SSSSNEKLEETLEEVEDVKNIMEDNEELVEEQGEQIDELEEKADDLEESAEELSDNSE |
| | SEQ ID NO: 203 |
| N60 | SSSSNEKLRETLREVEDVKNIMEDNRRLVERQGRQIDRLEEKADDLEKSAERLSDNSE |
| | SEQ ID NO: 204 |
| N61 | SSSSNEKLRETLREVEDVKNIMEDNRRLVERQGRQIDRLEEKADDLEESAERLSDNSE |
| | SEQ ID NO: 205 |
| N62 | SSSSNEKLRETLREVEDVKNIMEDNRRLVERQGRQIDRLEEKADDLERTAERLSDNSE |
| | SEQ ID NO: 206 |
| N63 | SSSSNEKLRETLREVEDVKNIMEDNRRLVERQGRQIDRLEEKADDLERAAERLSDNSE |
| | SEQ ID NO: 207 |
| N64 | SSSSNEKLRETLREVEDVKNIMEDNRRLVERQGRQIDRLEEKADDLEREAERLSDNSE |
| | SEQ ID NO: 208 |

In one embodiment, X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:156. The X1 domain of SEQ ID NO:156 is present in the full length VAMP2 redesign of SEQ ID NO:8 (Table 5). Residues present at the interface between VAMP2 and sc-t-SP in SEQ ID NO:156 are L8, L12, V15, V18, 121, M22, L28, V29, G33, 136, L39, L46, L53 (see highlighted residues below). Thus, in one embodiment, 1, 2, 3, 4, 5, 6, 7, 8, 9, 10, 11, 12, or all 13 of L8, L12, V15, V18, 121, M22, L28, V29, G33, 136, L39, L46, L53 are conserved (i.e., identical) in the polypeptide relative to SEQ ID NO:156 (see below).

SEQ ID NO: 156
SSSSNEKLRETLREVEDVKNIMEDNRRLVF
RQGRQIDRLEEKADDLERSAERLSDNSE

In another embodiment, the X2 domain comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:209-222 and 456, or selected from the group consisting of SEQ ID NO:213 and 214. The amino acid sequences of the X2 JMDs are provided in Table 3.

TABLE 3
KLKKKKKKKKKK SEQ ID NO: 209
KIKKKFFFKKFK SEQ ID NO: 210
RIRRRFFFRRFR SEQ ID NO: 211
KLTKYYEEKESK SEQ ID NO: 212
KLKRKYWWKNLK SEQ ID NO: 213
KLKRKYWWKNSK SEQ ID NO: 214
KIKKKFFFKKSK SEQ ID NO: 215
KIKKKWWWKKSK SEQ ID NO: 216
KIKKKYYYKKSK SEQ ID NO: 217
KIKKKSSSKKSK SEQ ID NO: 218
KIKKKYYYKKFK SEQ ID NO: 219
KIKKKSSSKKFK SEQ ID NO: 220
KIKKKWWWKKFK SEQ ID NO: 221
KLKKYYEEKQTK SEQ ID NO: 222
KAKRKYWWKNSK SEQ ID NO: 456

In a further embodiment, X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: SEQ ID NO: 223-234, or selected from the group consisting of SEQ ID NO:223 or 232. The amino acid sequences of the X3 TMDs is provided in Table 4.

TABLE 4
Native
MMIILGVICAIILIIIIVYFST SEQ ID NO: 223
VSV-G
FFFIIGLIIGLFLVLRVGIHLST SEQ ID NO: 224
Flu-HA
ILWISFAISCFLLCVVLLGFIST SEQ ID NO: 225
EGFR
IATGMVGALLLLLVVALGIGLEST SEQ ID NO: 226
PDGFR
AAVLVLLVIVIISLIVLVVIWST SEQ ID NO: 227
Syntaxin 1A
IMIIICCVILGIVIASTVGGIST SEQ ID NO: 228
polyVal
MMVVVVVVVVVVVVVVVVYFST SEQ ID NO: 229
polyIle
MMIIIIIIIIIIIIIIIIYFST SEQ ID NO: 230
polyLeu
MMLLLLLLLLLLLLLLLLYFST SEQ ID NO: 231
Native with C-terminal deletion
MMIILGVICAIILIIIIVYFS SEQ ID NO: 232
IMIIICCVILGIVIASTVGGIFA SEQ ID NO: 233
AAVLVLLVIVIISLIVLVVIWFA SEQ ID NO: 234

In one embodiment, the polypeptide comprises the genus B2-X1-X2-X3, wherein B2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:235.

(SEQ ID NO: 235)
SATAATAPPAAPAGEGGPPAPPP

Some v-SP designs contain a Pro-rich region (SATAATAPPAAPAGEGGPPAPPP, (SEQ ID NO: 235)) at the N terminus derived from native VAMP2, but this region is dispensable for fusion activity, and thus is optional in the present designs, and may be present or absent.

In another embodiment of this first aspect, the nucleic acid encodes a polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-37, 147, and 236-289, or at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8. In a further embodiment, the nucleic acid encodes an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:235 N-terminal to the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-37, 147, and 236-289.

In one embodiment of this first aspect, the nucleic acid encodes a fusion protein, comprising:
(a) the nucleic acid of any embodiment of this first aspect; and
(b) a nucleic acid domain encoding an amino acid sequence at least 90%, 91%, 92%, 93%, 94%, 95%, 96%, 97%, 98%, 99%, or 100% identical to the amino acid sequence of SEQ ID NO: 290 or 291;
optionally wherein the encoded polypeptide and the polypeptide domain are connected by an amino acid linker.

(SEQ ID NO: 290)
GVQVETISPGDGRTFPKRGQTCVVHYTGMLEDGKKFDSSRDRNK
PFKFMLGKQEVIRGWEEGVAQMSVGQRAKLTISPDYAYGATGHP
GIIPPHATLVEDVELLKLE
(SEQ ID NO: 291)
VAILWHEMWHEGLEEASRLYFGERNVKGMFEVLEPLHAMMERGP
QTLKETSENQAYGRDLMEAQEWCRKYMKSGNVKDLTQAWDLYYH
VERRIS

In these embodiments the fusion protein can be used for inducible binding to sc-t-SP (see below) in the presence of rapamycin. The domain of SEQ ID NO:290 is an FKBP domain that can bind to its cognate binding partner FRB domain (SEQ ID NO:291) fused to the N-terminus of inducible sc-t-SPs (e.g. SEQ ID NO 148) in the presence of rapamycin and induce fusion. FKBP domain fused to v-SPs and FRB domain fused to sc-t-SPs would function similarly if they were interchanged with each other. In one such embodiment, the nucleic acid encodes a fusion protein comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:147 and 244-258.

The sequences of these full length VAMP2 redesigned proteins are shown in Tables 5 and 6.

TABLE 5
>SEQ ID NO: 1
SSSSSEKLRETQAQVDEVVDIMRVNVDKVLERDQKLSELDDRADALQAGASQFETSAAKLKRK
YWWKNLKMMIILGVICAIILIIIIVYFST
> SEQ ID NO: 2
NLASNRRLQQTSEEVREVNDIMRVNVDKVLERDQKLSELDDRADALQAGASQFETSAAKLKRK
YWWKNLKMMIILGVICAIILIIIIVYFST
> SEQ ID NO: 3
NLASNRRLQQTQAQVDEVVDVMRDNRNLVDERDQKLSELDDRADALQAGASQFETSAAKLKRK
YWWKNLKMMIILGVICAIILIIIIVYFST
> SEQ ID NO: 4
NLASNRRLQQTQAQVDEVVDIMRVNVDKVLRQGEQIDRLEDRADALQAGASQFETSAAKLKRK
YWWKNLKMMIILGVICAIILIIIIVYFST
> SEQ ID NO: 5
NLASNRRLQQTQAQVDEVVDIMRVNVDKVLERDQKLSELDERADELEKSASQFETSAAKLKRK
YWWKNLKMMIILGVICAIILIIIIVYFST
> SEQ ID NO: 6
NLASNRRLQQTQAQVDEVVDIMRVNVDKVLERDQKLSELDDRADALQAGAERLEENATKLKRK
YWWKNLKMMIILGVICAIILIIIIVYFST
> SEQ ID NO: 7
NLASNRRLQQTQAQVDEVVDIMRVNVDKVLERDQKLSELDDRADALQAGASQFETSAAKLTKY
YEEKESKMMIILGVICAIILIIIIVYFST
> SEQ ID NO: 8
SSSSNEKLRETLREVEDVKNIMEDNRRLVERQGRQIDRLEEKADDLERSAERLSDNSEKLKRK
YWWKNSKMMIILGVICAIILIIIIVYFST
> SEQ ID NO: 9
SSSDSSKLRETEEETRDVIDIMRDNRRSVEERGRQIDRLEERADDLEDSAERLEENAKKLKRK
YWWKNSKMMIILGVICATILIIIIVYFST
> SEQ ID NO: 10
SSSSNEKLRETLREVEDVKNIMEDNRRLVERQGRQIDRLEEKADDLERSAERLSDNSEKLKRK
YWWKNSKMMIILGVICAIILIIIIVYFST
> SEQ ID NO: 11
SGSSNERLREVSKEAREVREMAMDVKEKIEEQGRKIEELEEKAESLKDSAERFDENAKKLKRK
YWWKNLKMMIILGVICAIILIIIIVYFST
> SEQ ID NO: 12
SGTTNEKLRKVSSEADEVKEMGMDVKEKVEEQGRKIEELEEKAEDLKDSAERFDENAKKLKRK
YWWKNLKMMIILGVICAIILIIIIVYFST
> SEQ ID NO: 13
SGSSSEKLRQISSEAEEVKEMGMDILKKIEEQGEKIERLEEKAESLKDSAERFADNAKKLKRK
YWWKNLKMMIILGVICAIILIIIIVYFST
> SEQ ID NO: 14
DGTSNERLRETSKEAREVRDMAMDNMKKVEEQGEKIEELEEKAEELKDSAERLDDNAKKLKRK
YWWKNLKMMIILGVICAIILIIIIVYFST
> SEQ ID NO: 15
DGTSNEKLRETSEQAREVRDMALDNKEKIEEQGEKIDRLEEKAESLKDSAERFAENAKKLKRK
YWWKNLKMMIILGVICAIILIIIIVYFST
> SEQ ID NO: 16
SEEMSKKLEETSKEVDEVLEIMEEIREMLEEQGRRIDRLEKKAEELEEGAEKFEELSEKLKRK
YWWKNLKMMIILGVICATILIIIIVYFST
> SEQ ID NO: 17
SEERKEKLEETLKEVDEVLEIMKENKEMLEEQGERLERLEEKAEELEEGAEKFEELAEKLKRK
YWWKNLKMMIILGVICAIILIIIIVYFST
> SEQ ID NO: 18
SKERSEKLKETMEEVEEVLEIMKEIRRMMEEQGERIDRLEEKAEELEEGAEKFEELAEKLKRK
YWWKNLKMMIILGVICAIILIIIIVYFST
> SEQ ID NO: 19
SSSSNEKLRETLREVEDVKNIMEDNRRLVERQGRQIDRLEEKADDLERSAERLSDNSEKLKRK
YWWKNLKMMIILGVICAIILIIIIVYFST
> SEQ ID NO: 20
SSSSNEKLRETLREVEDVKNIMEDNRRLVERQGRQIDRLEEKADDLERSAERLSDNSEKLKKK
KKKKKKKMMIILGVICAIILIIIIVYFST
> SEQ ID NO: 21
SSSSNEKLRETLREVEDVKNIMEDNRRLVERQGRQIDRLEEKADDLERSAERLSDNSEKLKRK
YWWKNLKAAVLVLLVIVIISLIVLVVIWST
> SEQ ID NO: 22
SSSSNEKLRETLREVEDVKNIMEDNRRLVERQGRQIDRLEEKADDLERSAERLSDNSEKLKRK
YWWKNLKMMVVVVVVVVVVVVVVVVYFST
> SEQ ID NO: 23
SSSSNEKLRETLREVEDVKNIMEDNRRLVERQGRQIDRLEEKADDLERSAERLSDNSEKLKRK
YWWKNLKMMIIIIIIIIIIIIIIIIYFST
> SEQ ID NO: 24
SSSSNEKLRETLREVEDVKNIMEDNRRLVERQGRQIDRLEEKADDLERSAERLSDNSEKLKKK
KKKKKKKAAVLVLLVIVIISLIVLVVIWST
> SEQ ID NO: 25
SSSSNEKLRETLREVEDVKNIMEDNRRLVERQGRQIDRLEEKADDLERSAERLSDNSEKLKKK
KKKKKKKMMVVVVVVVVVVVVVVVVYFST
> SEQ ID NO: 26
NLASNRRLQQTQAQVDEVVDIMRVNVDKVLERDQKLSELDDRADALQAGASQFETSAAKLKKK
KKKKKKKMMIILGVICAIILIIIIVYFST
> SEQ ID NO: 27
NLASNRRLQQTQAQVDEVVDIMRVNVDKVLERDQKLSELDDRADALQAGASQFETSAAKLKRK
YWWKNLKMMLLLLLLLLLLLLLLLLYFST
> SEQ ID NO: 28
NLASNRRLQQTQAQVDEVVDIMRVNVDKVLERDQKLSELDDRADALQAGASQFETSAAKLKRK
YWWKNLKMMVVVVVVVVVVVVVVVVYFST
> SEQ ID NO: 29
NLASNRRLQQTQAQVDEVVDIMRVNVDKVLERDQKLSELDDRADALQAGASQFETSAAKLKRK
YWWKNLKFFFIIGLIIGLFLVLRVGIHLST
> SEQ ID NO: 30
NLASNRRLQQTQAQVDEVVDIMRVNVDKVLERDQKLSELDDRADALQAGASQFETSAAKLKRK
YWWKNLKILWISFAISCFLLCVVLLGFIST
> SEQ ID NO: 31
NLASNRRLQQTQAQVDEVVDIMRVNVDKVLERDQKLSELDDRADALQAGASQFETSAAKLKRK
YWWKNLKIATGMVGALLLLLVVALGIGLFST
> SEQ ID NO: 32
NLASNRRLQQTQAQVDEVVDIMRVNVDKVLERDQKLSELDDRADALQAGASQFETSAAKLKRK
YWWKNLKIMIIICCVILGIVIASTVGGIST
> SEQ ID NO: 33
NLASNRRLQQTQAQVDEVVDIMRVNVDKVLERDQKLSELDDRADALQAGASQFETSAAKLKRK
YWWKNLKAAVLVLLVIVIISLIVLVVIWST
> SEQ ID NO: 34
SSSSNEKLRETLREVEDVKNIMEDNRRLVERQGRQIDRLEEKADDLERSAERLSDNSEKIKKK
FFFKKEKMMIILGVICAIILIIIIVYFST
> SEQ ID NO: 35
SSSSNEKLRETLREVEDVKNIMEDNRRLVERQGRQIDRLEEKADDLERSAERLSDNSERIRRR
FFFRRFRMMIILGVICAIILIIIIVYFST
> SEQ ID NO: 36
SSSSNEKLRETLREVEDVKNIMEDNRRLVERQGRQIDRLEEKADDLERSAERLSDNSEKIKKK
FFFKKFKAAVLVLLVIVIISLIVLVVIWST
> SEQ ID NO: 37
SSSSNEKLRETLREVEDVKNIMEDNRRLVERQGRQIDRLEEKADDLERSAERLSDNSEKIKKK
FFFKKFKMMVVVVVVVVVVVVVVVVFST
SEQ ID NO : 147
> SEQ ID NO: 147 (v-SP part is identical to WT-VAMP2, N001)
GVQVETISPGDGRTFPKRGQTCVVHYTGMLEDGKKFDSSRDRNKPFKFMLGKQEVIRGWEEGV
AQMSVGQRAKLTISPDYAYGATGHPGIIPPHATLVFDVELLKLEGGSGGSNLASNRRLQQTQA
QVDEVVDIMRVNVDKVLERDQKLSELDDRADALQAGASQFETSAAKLKRKYWWKNLKMMIILG
VICAIILIIIIVYFST
Coiled-coil domain (Bold font)
JMD: (Underlined)
TMD: (in italics)

TABLE 6

| # | amino acid sequence |
|---|---|
| N1 | SSSSNEKLRETLREVEDVKNIMEDNRRLVERQGRQIDRLEEKADDLERSAERLSDNSEK |
| | IKKKFFFKKSKMMIILGVICAIILIIIIVYEST |
| | SEQ ID NO: 236 |
| N2 | SSSSNEKLRETLREVEDVKNIMEDNRRLVERQGRQIDRLEEKADDLERSAERLSDNSEK |
| | IKKKWWWKKSKMMIILGVICAIILIIIIVYFST |
| | SEQ ID NO: 237 |
| N3 | SSSSNEKLRETLREVEDVKNIMEDNRRLVERQGRQIDRLEEKADDLERSAERLSDNSEK |
| | IKKKYYYKKSKMMIILGVICAIILIIIIVYFST |
| | SEQ ID NO: 238 |
| N4 | SSSSNEKLRETLREVEDVKNIMEDNRRLVERQGRQIDRLEEKADDLERSAERLSDNSEK |
| | IKKKSSSKKSKMMIILGVICAIILIIIIVYFST |
| | SEQ ID NO: 239 |
| N5 | SSSSNEKLRETLREVEDVKNIMEDNRRLVERQGRQIDRLEEKADDLERSAERLSDNSEK |
| | IKKKYYYKKFKMMIILGVICAIILIIIIVYFST |
| | SEQ ID NO: 240 |
| N6 | SSSSNEKLRETLREVEDVKNIMEDNRRLVERQGRQIDRLEEKADDLERSAERLSDNSEK |
| | IKKKSSSKKFKMMIILGVICAIILIIIIVYEST |
| | SEQ ID NO: 241 |
| N7 | SSSSNEKLRETLREVEDVKNIMEDNRRLVERQGRQIDRLEEKADDLERSAERLSDNSEK |
| | IKKKWWWKKFKMMIILGVICAIILIIIIVYFST |
| | SEQ ID NO: 242 |
| N8 | SATAATAPPAAPAGEGGPPAPPPSSSSNEKLRETSREVEEVNDIMRDNRNLVDRQGEQI |
| | DRLEERADELKDSAERLSENSKKLKKYYEEKQTKMMIILGVICAIILIIIIVYFST |
| | SEQ ID NO: 243 |
| N13 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETQAQVDEVVDIMRVNVDKVLERDQKLSELDDRADALQAGASQFETSAAKLKRKY |
| | WWKNLKMMIILGVICAIILIIIIVYFST |
| | SEQ ID NO: 244 |
| N14 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSNLASN |
| | RRLQQTLREVEDVKNIMRVNVDKVLERDQKLSELDDRADALQAGASQFETSAAKLKRKY |
| | WWKNLKMMIILGVICAIILIIIIVYFST |
| | SEQ ID NO: 245 |
| N15 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSNLASN |
| | RRLQQTQAQVDEVVDIMEDNRRLVEERDQKLSELDDRADALQAGASQFETSAAKLKRKY |
| | WWKNLKMMIILGVICAIILIIIIVYFST |
| | SEQ ID NO: 246 |
| N16 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVFDVELLKLEGGSGGSNLASN |
| | RRLQQTQAQVDEVVDIMRVNVDKVLRQGRQIDRLEDRADALQAGASQFETSAAKLKRKY |
| | WWKNLKMMIILGVICAIILIIIIVYFST |
| | SEQ ID NO: 247 |
| N17 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSNLASN |
| | RRLQQTQAQVDEVVDIMRVNVDKVLERDQKLSELDEKADDLERSASQFETSAAKLKRKY |
| | WWKNLKMMIILGVICAIILIIIIVYFST |
| | SEQ ID NO: 248 |
| N18 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSNLASN |
| | RRLQQTQAQVDEVVDIMRVNVDKVLERDQKLSELDDRADALQAGAERLSDNSEKLKRKY |
| | WWKNLKMMIILGVICAIILIIIIVYFST |
| | SEQ ID NO: 249 |
| N19 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSNLASN |
| | RRLQQTQAQVDEVVDIMRVNVDKVLERDQKLSELDDRADALQAGASQFETSAAKLKRKY |
| | WWKNSKMMIILGVICAIILIIIIVYFST |
| | SEQ ID NO: 250 |
| N20 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETLREVEDVKNIMEDNRRLVERQGRQIDRLEEKADDLERSAERLSDNSEKLKRKY |
| | WWKNSKMMIILGVICAIILIIIIVYFST |
| | SEQ ID NO: 251 |
| N21 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSNLASN |
| | RRLQQTLREVEDVKNIMEDNRRLVERQGRQIDRLEEKADDLERSAERLSDNSEKLKRKY |
| | WWKNSKMMIILGVICAIILIIIIVYFST |
| | SEQ ID NO: 252 |
| N22 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETQAQVDEVVDIMEDNRRLVERQGRQIDRLEEKADDLERSAERLSDNSEKLKRKY |
| | WWKNSKMMIILGVICAIILIIIIVYFST |
| | SEQ ID NO: 253 |
| N23 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETLREVEDVKNIMRVNVDKVLRQGRQIDRLEEKADDLERSAERLSDNSEKLKRKY |
| | WWKNSKMMIILGVICAIILIIIIVYFST |
| | SEQ ID NO: 254 |
| N24 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETLREVEDVKNIMEDNRRLVEERDQKLSELDEKADDLERSAERLSDNSEKLKRKY |
| | WWKNSKMMIILGVICAIILIIIIVYFST |
| | SEQ ID NO: 255 |
| N25 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETLREVEDVKNIMEDNRRLVERQGRQIDRLEDRADALQAGAERLSDNSEKLKRKY |
| | WWKNSKMMIILGVICAIILIIIIVYEST |
| | SEQ ID NO: 256 |
| N26 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETLREVEDVKNIMEDNRRLVERQGRQIDRLEEKADDLERSASQFETSAAKLKRKY |
| | WWKNSKMMIILGVICAIILIIIIVYFST |
| | SEQ ID NO: 257 |
| N27 | GVQVETISPGDGRTFPKRGQTCVVHYTGMLEDGKKFDSSRDRNKPFKFMLGKQEVIRGW |
| | EEGVAQMSVGQRAKLTISPDYAYGATGHPGIIPPHATLVEDVELLKLEGGSGGSSSSSN |
| | EKLRETLREVEDVKNIMEDNRRLVERQGRQIDRLEEKADDLERSAERLSDNSEKLKRKY |
| | WWKNLKMMIILGVICAIILIIIIVYFST |
| | SEQ ID NO: 258 |
| N34 | SEEEEKKKEELKKKLKEALEEAKKAKELAKKALELAERQGRQIDRLEEKADDLERSAER |
| | LSDNSEKLKRKYWWKNSKMMIILGVICAIILIIIIVYES |
| | SEQ ID NO: 259 |
| N35 | EEEKEKKKEELKEKAKKALEEAKKTKELAKEALELAERQGRQIDRLEEKADDLERSAER |
| | LSDNSEKLKRKYWWKNSKMMIILGVICAIILIIIIVYES |
| | SEQ ID NO: 260 |
| N36 | SLEAEKKEKEEKEKKKKILELLKELLEETEELKEEAEEIKREVERQGRQIDRLEEKADD |
| | LERSAERLSDNSEKLKRKYWWKNSKMMIILGVICAIILIIIIVYFS |
| | SEQ ID NO: 261 |
| N37 | ELEEELKKKEEEEKRKEILELLKELLEETEELKEEAEEIKEEVERQGRQIDRLEEKADD |
| | LERSAERLSDNSEKLKRKYWWKNSKMMIILGVICAIILIIIIVYFS |
| | SEQ ID NO: 262 |
| N38 | SSSSNEKARETLREVEDVKNIMEDNRRLVERQGRQIDRLEEKADDLERSAERLSDNSEK |
| | LKRKYWWKNSKMMIILGVICAIILIIIIVYFST |
| | SEQ ID NO: 263 |
| N39 | SSSSNEKLRETAREVEDVKNIMEDNRRLVERQGRQIDRLEEKADDLERSAERLSDNSEK |
| | LKRKYWWKNSKMMIILGVICAIILIIIIVYEST |
| | SEQ ID NO: 264 |
| N40 | SSSSNEKLRETLREAEDVKNIMEDNRRLVERQGRQIDRLEEKADDLERSAERLSDNSEK |
| | LKRKYWWKNSKMMIILGVICAIILIIIIVYFST |
| | SEQ ID NO: 265 |
| N41 | SSSSNEKLRETLREVEDAKNIMEDNRRLVERQGRQIDRLEEKADDLERSAERLSDNSEK |
| | LKRKYWWKNSKMMIILGVICAIILIIIIVYFST |
| | SEQ ID NO: 266 |
| N42 | SSSSNEKLRETLREVEDVKNAMEDNRRLVERQGRQIDRLEEKADDLERSAERLSDNSEK |
| | LKRKYWWKNSKMMIILGVICAIILIIIIVYEST |
| | SEQ ID NO: 267 |
| N43 | SSSSNEKLRETLREVEDVKNIAEDNRRLVERQGRQIDRLEEKADDLERSAERLSDNSEK |
| | LKRKYWWKNSKMMIILGVICAIILIIIIVYFST |
| | SEQ ID NO: 268 |
| N44 | SSSSNEKLRETLREVEDVKNIMEDNRRAVERQGRQIDRLEEKADDLERSAERLSDNSEK |
| | LKRKYWWKNSKMMIILGVICAIILIIIIVYFST |
| | SEQ ID NO: 269 |
| N45 | SSSSNEKLRETLREVEDVKNIMEDNRRLAERQGRQIDRLEEKADDLERSAERLSDNSEK |
| | LKRKYWWKNSKMMIILGVICAIILIIIIVYEST |
| | SEQ ID NO: 270 |
| N46 | SSSSNEKLRETLREVEDVKNIMEDNRRLVERQARQIDRLEEKADDLERSAERLSDNSEK |
| | LKRKYWWKNSKMMIILGVICAIILIIIIVYFST |
| | SEQ ID NO: 271 |
| N47 | SSSSNEKLRETLREVEDVKNIMEDNRRLVERQGRQADRLEEKADDLERSAERLSDNSEK |
| | LKRKYWWKNSKMMIILGVICAIILIIIIVYFST |
| | SEQ ID NO: 272 |
| N48 | SSSSNEKLRETLREVEDVKNIMEDNRRLVERQGRQIDRAEEKADDLERSAERLSDNSEK |
| | LKRKYWWKNSKMMIILGVICAIILIIIIVYEST |
| | SEQ ID NO: 273 |
| N49 | SSSSNEKLRETLREVEDVKNIMEDNRRLVERQGRQIDRLEEKADDAERSAERLSDNSEK |
| | LKRKYWWKNSKMMIILGVICAIILIIIIVYEST |
| | SEQ ID NO: 274 |
| N50 | SSSSNEKLRETLREVEDVKNIMEDNRRLVERQGRQIDRLEEKADDLERSAERASDNSEK |
| | LKRKYWWKNSKMMIILGVICAIILIIIIVYEST |
| | SEQ ID NO: 275 |
| N51 | SSSSNEKLRETLREVEDVKNIMEDNRRLVERQGRQIDRLEEKADDLERSAERLSDNSEK |
| | AKRKYWWKNSKMMIILGVICAIILIIIIVYFST |
| | SEQ ID NO: 276 |
| N52 | SSSSNEKARETAREAEDVKNIMEDNRRLVERQGRQIDRLEEKADDLERSAERLSDNSEK |
| | LKRKYWWKNSKMMIILGVICAIILIIIIVYFST |
| | SEQ ID NO: 277 |
| N53 | SSSSNEKARETAREVEDVKNIMEDNRRLVERQGRQIDRLEEKADDLERSAERLSDNSEK |
| | LKRKYWWKNSKMMIILGVICAIILIIIIVYFST |
| | SEQ ID NO: 278 |
| N54 | SSSSNEKARETAREAEDAKNIMEDNRRLVERQGRQIDRLEEKADDLERSAERLSDNSEK |
| | LKRKYWWKNSKMMIILGVICAIILIIIIVYFST |
| | SEQ ID NO: 279 |
| N55 | SSSSNEKARETAREAEDAKNAMEDNRRLVERQGRQIDRLEEKADDLERSAERLSDNSEK |
| | LKRKYWWKNSKMMIILGVICAIILIIIIVYFST |
| | SEQ ID NO: 280 |
| N56 | SSSSNEKARETAREAEDAKNAAEDNRRLVERQGRQIDRLEEKADDLERSAERLSDNSEK |
| | LKRKYWWKNSKMMIILGVICAIILIIIIVYFST |
| | SEQ ID NO: 281 |
| N57 | SSSSNEKARETLREVEDVKNIAEDNRRLVERQGRQIDRAEEKADDLERSAERLSDNSEK |
| | LKRKYWWKNSKMMIILGVICAIILIIIIVYFST |
| | SEQ ID NO: 282 |
| N58 | SSSSNEKLKETLKEVEDVKNIMEDNKKLVEKQGKQIDKLEEKADDLEKSAEKLSDNSEK |
| | LKRKYWWKNSKMMIILGVICAIILIIIIVYFST |
| | SEQ ID NO: 283 |
| N59 | SSSSNEKLEETLEEVEDVKNIMEDNEELVEEQGEQIDELEEKADDLEESAEELSDNSEK |
| | LKRKYWWKNSKMMIILGVICAIILIIIIVYFST |
| | SEQ ID NO: 284 |
| N60 | SSSSNEKLRETLREVEDVKNIMEDNRRLVERQGRQIDRLEEKADDLEKSAERLSDNSEK |
| | LKRKYWWKNSKMMIILGVICAIILIIIIVYFST |
| | SEQ ID NO: 285 |
| N61 | SSSSNEKLRETLREVEDVKNIMEDNRRLVERQGRQIDRLEEKADDLEESAERLSDNSEK |
| | LKRKYWWKNSKMMIILGVICAIILIIIIVYFST |
| | SEQ ID NO: 286 |
| N62 | SSSSNEKLRETLREVEDVKNIMEDNRRLVERQGRQIDRLEEKADDLERTAERLSDNSEK |
| | LKRKYWWKNSKMMIILGVICAIILIIIIVYFST |
| | SEQ ID NO: 287 |
| N63 | SSSSNEKLRETLREVEDVKNIMEDNRRLVERQGRQIDRLEEKADDLERAAERLSDNSEK |
| | LKRKYWWKNSKMMIILGVICAIILIIIIVYFST |
| | SEQ ID NO: 288 |
| N64 | SSSSNEKLRETLREVEDVKNIMEDNRRLVERQGRQIDRLEEKADDLEREAERLSDNSEK |
| | LKRKYWWKNSKMMIILGVICAIILIIIIVYFST |
| | SEQ ID NO: 289 |

In a further embodiment of all aspects of the disclosure, the nucleic acid encodes a polypeptide that further comprises a signal peptide at its amino-terminus. Any signal peptide may be used as suitable for an intended purpose. The signal peptide may be directly linked to the polypeptide, or may be connected via an amino acid linker. In some embodiments, the signal peptide comprises the amino acid sequence selected from the group consisting of SEQ ID NO: 292-309. The amino acid sequence of these exemplary signal peptides are provided in Table 7.

TABLE 7

| SEQ | | |
|---|---|---|
| ID | | |
| NO | Name | Sequence |
| 292 | Bovine | MDSKGSSQKGSRLLLLLVVSN |
| | prolactin | LLLCQGVVST |
| 293 | Human | MYRMQLLSCIALSLALVINS |
| | interleukin-2 | |
| 294 | Human OSM | MGVLLTQRTLLSLVLALLFPS |
| | | MASM |
| 295 | VSV-G | MKCLLYLAFLFIGVNC |
| 296 | Mouse Ig Kappa | METDTLLLWVLLLWVPGSTGD |
| 297 | Mouse Ig Heavy | MGWSCIILFLVATATGVHS |
| 298 | BM40 | MRAWIFFLLCLAGRALA |
| 299 | Secrecon | MWWRLWWLLLLLLLLWPMVWA |
| 300 | Human IgKVIII | MDMRVPAQLLGLLLLWLRGARC |
| 301 | CD33 | MPLLLLLPLLWAGALA |
| 302 | tPA | MDAMKRGLCCVLLLCGAVEVS |
| | | PS |
| 303 | Human | MAFLWLLSCWALLGTTFG |
| | Chymotrypsinogen | |
| 304 | Human | MNLLLILTFVAAAVA |
| | trypsinogen-2 | |
| 305 | Silkworm | MKPIFLVLLVVTSAYA |
| | Fibroin LC | |
| 306 | Gaussia luc | MGVKVLFALICIAVAEA |
| 307 | Albumin (HSA) | MKWVT FISLLESSAYS |
| 308 | Influenza | MKTIIALSYIFCLVLG |
| | Haemagglutinin | |
| 309 | Human insulin | MALWMRLLPLLALLALWGP |
| | | DPAAA |

In a further embodiment of any aspect of the disclosure, the nucleic acid comprises an expression vector comprising the nucleic acid operatively linked to a control sequence, such as a promoter.

### SNAP25-Redesigns

In a second aspect, the disclosure provides nucleic acids encoding SNAP25-redesigned polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:310-316, wherein X1 is an amino acid linker.

In this second aspect, the disclosure provides redesigned SNAP25 proteins in which one of the two coiled-coil domains is an anti-parallel coiled-coil, as described above. When combined with v-SP and native or redesigned Syn1A, redesigned SNAP25 is capable of inducing cell-cell fusion when displayed on the surface of mammalian cells, or liposome fusion when displayed on the surface of liposomes. For membrane fusion to occur, v-SP is presented on one membrane and SNAP25 and Syn1A on the other.

The amino acid sequences of SEQ ID NO:310-316 are provided in Table 9.

TABLE 8
>SEQ ID NO: 310
AEDADLEKQKQEEEKRGETLKDESLEATRKMVNMVREAREMAMRNGELLESQGEKLDRIEEKADRMETKLDE
ADEDLKKIEG-X1-
DARENEMDENLEQVSGIIGNLRHMALDMGNEIDTQNRQIDRIMEKADSAKTRIDEANQRATKMLGSG
> SEQ ID NO: 311
AEDADSLAQQQQEEQRGSTLIDESLEATRKMKEMVEEAVRMAMDNGELLRSQGEKLDRIEEKADRMESLLDE
ADENLDKIEG-X1-
DARENEMDENLEQVSGIIGNLRHMALDMGNEIDTQNRQIDRIMEKADSAKTRIDEANQRATKMLGSG
> SEQ ID NO: 312
AEDADMRNELEEMQRRADQLADESLESTRRMLQLVEESKDAGIRTLVMLDEQGEQLERIEEGMDQINKDMKE
AEKNLADLGK-X1-
PSYIREVNNSEKEKEINEGLGRVDQQVQELKDMAVVMGEKVDEQNEKIDRINEKADKNEQRVNDLTKEAEKL
LNSG
> SEQ ID NO: 313
AEDADMRNELEEMQRRADQLADESLESTRRMLQLVEESKDAGIRTLVMLDEQGEQLERIEEGMDQINKDMKE
AEKNLADLGK-X1-
SSFIRRVNGSEREREIDRGLERVDQQVKELKDMARVMGDKTDEQGEKIDRIEEKADRNEERVEKLVKEAKEL
LESG
> SEQ ID NO: 314
DARENEMDENLEQVSGIIGNLRHMALDMGNEIDTQNRQIDRIMEKADSAKTRIDEANQRATKMLGSG
X1-EELKDLEKEGKELKELVEELDREVKELKESMEKLKEMTEEAAELSSQALEIMRRTRKLSEELLKEAKE
EEEEEEEEEEEEE
> SEQ ID NO: 315
DARENEMDENLEQVSGIIGNLRHMALDMGNEIDTQNRQIDRIMEKADSAKTRIDEANQRATKMLGSG-X1-
EELKKLEKEGEKLKELVEELDREIKELKEGMERLREMFEEAAKLSEEALEIMRRTRKLSEEELEEAKQKAKE
QEKEKALKEK
> SEQ ID NO: 316
NKREEEIDKGLDRVGEIISKLNEMAREMGEKIEEQNQKISEIEKKADEAIEKVEKLIKDAEKLLGSG-X1-
EELKKLEKEGEKLKELVEELDREIKELKEGMERLREMFEEAAKLSEEALEIMRRTRKLSEEELEEAKQKAKE
QEKEKALKEK

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:314-316, wherein X1 is an amino acid linker.

The XI linker may be any linker suitable for an intended purpose. In some embodiments, the amino acid linker is a GS-rich linker of less than 20, less than 15, or less than 10 amino acids in length. As used here, “GS-rich” means at least 50% G or S residues.

In a further embodiment, the nucleic acid encodes a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:38-44. In a further embodiment, the nucleic acid encodes a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:42-44.

The amino acid sequence of SEQ ID NO:38-44 are provided in Table 9.

TABLE 9
>SEQ ID NO: 38
AEDADLEKQKQEEEKRGETLKDESLEATRKMVNMVREAREMAMRNGELLESQGEKLDRIEEKADRMETKLDE
ADEDLKKIEG FSGLSVSPSNKLKSSDAYKKAWGNNQDGVVASQPARVVDEREQMAISGGFIRRVTNDARENE
MDENLEQVSGIIGNLRHMALDMGNEIDTQNRQIDRIMEKADSAKTRIDEANQRATKMLGSG
> SEQ ID NO: 39
AEDADSLAQQQQEEQRGSTLIDESLEATRKMKEMVEEAVRMAMDNGELLRSQGEKLDRIEEKADRMESLLDE
ADENLDKIEGFSGLSVSPSNKLKSSDAYKKAWGNNQDGVVASQPARVVDEREQMAISGGFIRRVTNDARENE
MDENLEQVSGIIGNLRHMALDMGNEIDTQNRQIDRIMEKADSAKTRIDEANQRATKMLGSG
> SEQ ID NO: 40
AEDADMRNELEEMQRRADQLADESLESTRRMLQLVEESKDAGIRTLVMLDEQGEQLERIEEGMDQINKDMKE
AEKNLADLGKFSGLSVSPSNKLKSSDAYKKAWGNNQDGVVASQPARVVDEREQMAISPSYIREVNNSEKEKE
INEGLGRVDQQVQELKDMAVVMGEKVDEQNEKIDRINEKADKNEQRVNDLTKEAEKLLNSG
> SEQ ID NO: 41
AEDADMRNELEEMQRRADQLADESLESTRRMLQLVEESKDAGIRTLVMLDEQGEQLERIEEGMDQINKDMKE
AEKNLADLGKFSGLSVSPSNKLKSSDAYKKAWGNNQDGVVASQPARVVDEREQMAISSSFIRRVNGSERERE
IDRGLERVDQQVKELKDMARVMGDKTDEQGEKIDRIEEKADRNEERVEKLVKEAKELLESG
> SEQ ID NO: 42
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEAEEEE
> SEQ ID NO: 43
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEK
> SEQ ID NO: 44
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEK

In a further embodiment, the nucleic acids of this second aspect encode a polypeptide that further comprises a signal peptide at its amino-terminus. Any signal peptide may be used as suitable for an intended purpose. The signal peptide may be directly linked to the polypeptide, or may be connected via an amino acid linker. In some embodiments, the signal peptide comprises the amino acid sequence selected from the group consisting of SEQ ID NO:292-309. The amino acid sequence of these exemplary signal peptides are provided in Table 7.

In a further embodiment, the nucleic acids of the second aspect comprise an expression vector comprising the nucleic acid operatively linked to a control sequence, such as a promoter.

### Syn1A-Redesigns

In a third aspect, the disclosure provides nucleic acids encoding Syn1A-redesigned polypeptide comprising the formula X1-X2-X3, wherein:
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 317-330;
X2 comprises a juxtamembrane domain (JMD); and
X3 comprises a transmembrane domain (TMD).

This aspect provides redesigned Syntaxin 1A (Syn1A) variants as described above. When combined with v-SP and native or redesigned SNAP25, redesigned Syn1A is capable of inducing cell-cell fusion when displayed on the surface of mammalian cells, or liposome fusion when displayed on the surface of liposomes. For membrane fusion to occur, v-SP is presented on one membrane and Syn1A and SNAP25 on the other.

The amino acid sequence of SEQ ID NO:317-330 are provide in Table 10.

TABLE 10
>SEQ ID NO: 317
SIEMEELSEIETRHSEIIKLENSIRELHDMEMDMAMLVESQGEMIDRIEYNVEHAVDYVERAVSD
> SEQ ID NO: 318
SISKQALKRLEERHSEIIKLENSIRELHDMEMDMAMLVESQGEMIDRIEYNVEHAVDYVERAVSD
> SEQ ID NO: 319
SISKQALSEIETNNEQVIKLENSIRELHDMFMDMAMLVESQGEMIDRIEYNVEHAVDYVERAVSD
> SEQ ID NO: 320
SISKQALSEIETRHSEIRRLLESIRELHDMEMDMAMLVESQGEMIDRIEYNVEHAVDYVERAVSD
> SEQ ID NO: 321
SISKQALSEIETRHSEIIKLENSVEEMHDMEMDMAMLVESQGEMIDRIEYNVEHAVDYVERAVSD
> SEQ ID NO: 322
SISKQALSEIETRHSEIIKLENSIRELKDMARDMAMLVESQGEMIDRIEYNVEHAVDYVERAVSD
> SEQ ID NO: 323
SISKQALSEIETRHSEIIKLENSIRELHDMEMRLGDMVESQGEMIDRIEYNVEHAVDYVERAVSD
> SEQ ID NO: 324
SISKQALSEIETRHSEIIKLENSIRELHDMEMDMAMLVDEQGEMIDRIEYNVEHAVDYVERAVSD
> SEQ ID NO: 325
SISKQALSEIETRHSEIIKLENSIRELHDMEMDMAMLVESQGEKISRIEYNVEHAVDYVERAVSD
> SEQ ID NO: 326
SISKQALSEIETRHSEIIKLENSIRELHDMEMDMAMLVESQGEMIDRIEERVEHAVDYVERAVSD
> SEQ ID NO: 327
SISKQALSEIETRHSEIIKLENSIRELHDMEMDMAMLVESQGEMIDRIEYNVEAAEAYVERAVSD
> SEQ ID NO: 328
SISKQALSEIETRHSEIIKLENSIRELHDMEMDMAMLVESQGEMIDRIEYNVEHAVDGVKAAVSD
> SEQ ID NO: 329
SISKQALSEIETRHSEIIKLENSIRELHDMEMDMAMLVESQGEMIDRIEYNVEHAVDYVERAKDN
> SEQ ID NO: 330
SISKQALSEIETRHSEIIKLENSIRELHDMFMDMAMLVESQGEMIDRIEYNVEHAVDYVERAVSD

In one embodiment of this third aspect, X2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:331-332.

SEQ ID NO: 331
TKKAVKYQSKARRKK
SEQ ID NO: 332
TKKAVKYQSRRRRRR

In a further embodiment, X3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: SEQ ID NO: 223-234, or selected from the group consisting of SEQ ID NO:223 or 232. The amino acid sequences of the X3 TMDs is provided in Table 4.

In another embodiment of this third aspect, the nucleic acid encodes a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:45-63. The amino acid sequences of SEQ ID NO:45-63 are provided in Table 11.

TABLE 11
>SEQ ID NO: 45
SIEMEELSEIETRHSEIIKLENSIRELHDMEMDMAMLVESQGEMIDRIEYNVEHAVDYVERAVSDTKKAVKY
QSKARRKKIMIIICCVILGIVIASTVGGIFA
> SEQ ID NO: 46
SISKQALKRLEERHSEIIKLENSIRELHDMEMDMAMLVESQGEMIDRIEYNVEHAVDYVERAVSDTKKAVKY
QSKARRKKIMIIICCVILGIVIASTVGGIFA
> SEQ ID NO: 47
SISKQALSEIETNNEQVIKLENSIRELHDMFMDMAMLVESQGEMIDRIEYNVEHAVDYVERAVSDTKKAVKY
QSKARRKKIMIIICCVILGIVIASTVGGIFA
> SEQ ID NO: 48
SISKQALSEIETRHSEIRRLLESIRELHDMEMDMAMLVESQGEMIDRIEYNVEHAVDYVERAVSDTKKAVKY
QSKARRKKIMIIICCVILGIVIASTVGGIFA
> SEQ ID NO: 49
SISKQALSEIETRHSEIIKLENSVEEMHDMEMDMAMLVESQGEMIDRIEYNVEHAVDYVERAVSDTKKAVKY
QSKARRKKIMIIICCVILGIVIASTVGGIFA
> SEQ ID NO: 50
SISKQALSEIETRHSEIIKLENSIRELKDMARDMAMLVESQGEMIDRIEYNVEHAVDYVERAVSDTKKAVKY
QSKARRKKIMIIICCVILGIVIASTVGGIFA
> SEQ ID NO: 51
SISKQALSEIETRHSEIIKLENSIRELHDMEMRLGDMVESQGEMIDRIEYNVEHAVDYVERAVSDTKKAVKY
QSKARRKKIMIIICCVILGIVIASTVGGIFA
> SEQ ID NO: 52
SISKQALSEIETRHSEIIKLENSIRELHDMEMDMAMLVDEQGEMIDRIEYNVEHAVDYVERAVSDTKKAVKY
QSKARRKKIMIIICCVILGIVIASTVGGIFA
> SEQ ID NO: 53
SISKQALSEIETRHSEIIKLENSIRELHDMEMDMAMLVESQGEKISRIEYNVEHAVDYVERAVSDTKKAVKY
QSKARRKKIMIIICCVILGIVIASTVGGIFA
> SEQ ID NO: 54
SISKQALSEIETRHSEIIKLENSIRELHDMFMDMAMLVESQGEMIDRIEERVEHAVDYVERAVSDTKKAVKY
QSKARRKKIMIIICCVILGIVIASTVGGIFA
> SEQ ID NO: 55
SISKQALSEIETRHSEIIKLENSIRELHDMFMDMAMLVESQGEMIDRIEYNVEAAEAYVERAVSDTKKAVKY
QSKARRKKIMIIICCVILGIVIASTVGGIFA
> SEQ ID NO: 56
SISKQALSEIETRHSEIIKLENSIRELHDMEMDMAMLVESQGEMIDRIEYNVEHAVDGVKAAVSDTKKAVKY
QSKARRKKIMIIICCVILGIVIASTVGGIFA
> SEQ ID NO: 57
SISKQALSEIETRHSEIIKLENSIRELHDMEMDMAMLVESQGEMIDRIEYNVEHAVDYVERAKDNTKKAVKY
QSKARRKKIMIIICCVILGIVIASTVGGIFA
> SEQ ID NO: 58
SISKQALSEIETRHSEIIKLENSIRELHDMEMDMAMLVESQGEMIDRIEYNVEHAVDYVERAKDNTKKAVKY
QSKARRKKIMIIICCVILGIVIASTVGGIFA
> SEQ ID NO: 59
SISKQALSEIETRHSEIIKLENSIRELHDMEMDMAMLVESQGEMIDRIEYNVEHAVDYVERAVSDTKKAVKY
QSKARRKKFFFIIGLIIGLFLVLRVGIHLFA
> SEQ ID NO: 60
SISKQALSEIETRHSEIIKLENSIRELHDMEMDMAMLVESQGEMIDRIEYNVEHAVDYVERAVSDTKKAVKY
QSKARRKKILWISFAISCFLLCVVLLGFIFA
> SEQ ID NO: 61
SISKQALSEIETRHSEIIKLENSIRELHDMEMDMAMLVESQGEMIDRIEYNVEHAVDYVERAVSDTKKAVKY
QSKARRKKIATGMVGALLLLLVVALGIGLFFA
> SEQ ID NO: 62
SISKQALSEIETRHSEIIKLENSIRELHDMEMDMAMLVESQGEMIDRIEYNVEHAVDYVERAVSDTKKAVKY
QSKARRKKMMIILGVICAIILIIIIVYFFA
> SEQ ID NO: 63
SISKQALSEIETRHSEIIKLENSIRELHDMEMDMAMLVESQGEMIDRIEYNVEHAVDYVERAVSDTKKAVKY
QSKARRKKAAVLVLLVIVIISLIVLVVIWFA
Coiled-coil domain (bold font)
JMD: (underlined)
TMD: (italicized)

In a further embodiment, the nucleic acids of this third aspect encode a polypeptide that further comprises a signal peptide at its amino-terminus. Any signal peptide may be used as suitable for an intended purpose. The signal peptide may be directly linked to the polypeptide, or may be connected via an amino acid linker. In some embodiments, the signal peptide comprises the amino acid sequence selected from the group consisting of SEQ ID NO:292-309. The amino acid sequence of these exemplary signal peptides are provided in Table 7.

In a further embodiment, the nucleic acids of the second aspect comprise an expression vector comprising the nucleic acid operatively linked to a control sequence, such as a promoter.

sc-t-SP Designs

In a fourth aspect, the disclosure provides nucleic acids encoding single-chain t-SNARE-like proteins (sc-t-SPs) comprising the formula X1-X2-X3, wherein:
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of the first set bold font residues in SEQ ID NO: 333-425, wherein the non-highlighted residues are amino acid linkers that may be substituted with any other amino acid linker;
X2 comprises a juxtamembrane domain (JMD); and
X3 comprises a transmembrane domain (TMD).

In this fourth aspect, the disclosure provides single-chain t-SNARE-like proteins (sc-t-SPs) which fuse the C-terminus of redesigned SNAP25 to the N-terminus of Syn1A sequences as described below. The sc-t-SP designs have three coiled-coil domains, and one of them is anti-parallel as it is derived from the redesigned SNAP25 described above. The sc-t-SP designs can bind to v-SPs and form a four-helix bundle like the native SNARE complex, though one helix is anti-parallel unlike the native SNARE complex. When combined with v-SP, sc-t-SPs are capable of inducing membrane fusion in mammalian cells or liposomes. The sc-t-SP should be present on one membrane and v-SP on the other to induce membrane fusion. The amino acid sequences of the X1 domains of SEQ ID NO:333-425 are provided in Tables 12 and 13.

TABLE 12
>SEQ ID NO: 333
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEAEEEEGGSGGSGGSSISKQALSEIETRHSEIIKLENSIRELHDMEMDMAMLVESQGEMIDRIEYNV
EHAVDYVERAVSD
> SEQ ID NO: 334
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSD
> SEQ ID NO: 335
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEGGSGGSGGSSISKQALSEIETRHSEIIKLENSIRELHDMEMDMAMLVESQGEMIDRIEYNVEHAVD
YVERAVSD
> SEQ ID NO: 336
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EGGSGGSGGSSISKQALSEIETRHSEIIKLENSIRELHDMEMDMAMLVESQGEMIDRIEYNVEHAVDYVERA
VSD
> SEQ ID NO: 337
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKGGSG
GSGGSSISKQALSEIETRHSEIIKLENSIRELHDMEMDMAMLVESQGEMIDRIEYNVEHAVDYVERAVSD
> SEQ ID NO: 338
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELGGSGGSGGS
SISKQALSEIETRHSEIIKLENSIRELHDMEMDMAMLVESQGEMIDRIEYNVEHAVDYVERAVSD
> SEQ ID NO: 339
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKGGSGGSGGSSISKQ
ALSEIETRHSEIIKLENSIRELHDMEMDMAMLVESQGEMIDRIEYNVEHAVDYVERAVSD
> SEQ ID NO: 340
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEGGSGGSGGSALSEIETRHSEIIKLENSIRELHDMEMDMAMLVESQGEMIDRIEYNVEHAVDYVERA
VSD
> SEQ ID NO: 341
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EGGSGGSGGSALSEIETRHSEIIKLENSIRELHDMFMDMAMLVESQGEMIDRIEYNVEHAVDYVERAVSD
> SEQ ID NO: 342
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKGGSG
GSGGSALSEIETRHSEIIKLENSIRELHDMEMDMAMLVESQGEMIDRIEYNVEHAVDYVERAVSD
> SEQ ID NO: 343
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELGGSGGSGGS
ALSEIETRHSEIIKLENSIRELHDMEMDMAMLVESQGEMIDRIEYNVEHAVDYVERAVSD
> SEQ ID NO: 344
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKEELAKVEERHKQIQALLDKIEELYEMEKEMSEKISEQGQKIDRIEEKV
SKASEHVSKGVED
> SEQ ID NO: 345
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEVEKRHKQILELEEKIKELYEMEKEMSEKIEKQGQKIDRIDDKV
SEAKKHVEKAVED
> SEQ ID NO: 346
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 347
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEAVKKSLAAKEERHKQILELLEKIKELHEMFKELSEKIEKQGQKIDRIEDKV
SKASEHVSKGVED
> SEQ ID NO: 348
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEAVKKELAAIEERHEQILELLKKIEELYEMFKELSEKIEKQGQKIDRIEKKV
SEASRHVSKAVED
> SEQ ID NO: 349
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKDLAAIEERHQQILELEEKIKELHEMFKEMSEKISEQMQKIDRIEEKV
SKASEHVSKGVED
> SEQ ID NO: 350
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVRRELAAIEERHRQILELLEKIEELHEMFKEMSEKISKQMEKIDRIDDRV
SEASRHVEKGVED
> SEQ ID NO: 351
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKAVKEELANIENRHKQIDALYEKIKELHEMFLEMSERIEAQLQKIDRIDDKV
SKAKAHVEKGVED
> SEQ ID NO: 352
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMFMDMAMLVESQGEMIDRIEYNV
EHAVDYVERAVSD
> SEQ ID NO: 353
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSALSEIETRHSEIIKLENSIRELHDMEMDMAMLVESQGEMIDRIEYNVEHAVD
YVERAVSD
> SEQ ID NO: 354
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSD
> SEQ ID NO: 355
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSD
> SEQ ID NO: 356
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSD
> SEQ ID NO: 357
NEREKEIDEGLERVGELISKLKELAREMSEKIEEQNQKLSEIDKKAEEAIKLLEKANASAKKLLEKPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 358
NEREKEIEEGLERVGELISELKEMAREMSEKIEEQNKKLDEISKKADEAIKLLEKANKGAEELLKKPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 359
NEREKEIDEGLEKIGELISKLKEMAREMSEKIEEQNEKLDEIDKKADEAIKLLEEANKKAEKLLKKKGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 360
NEREKEIEEGLERIGELISKLKELAREMSEKIEEQNEKLSEISEKADEAIKLLEKANASAQKLLEKPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 361
NPREEEIDKGLEEIGKLISELKELAREMSEKIEEQNEKISEIDEKAKEAIELLKKANEKAKELLEKEGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 362
SPREKEIDEGLERVSELVKKLKELAEKMKEMIEEQGRRIERIERKAEEAKERIEKLNEKAEKLLEDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 363
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 364
SEREKEIDEGLEKVSEIVKELKEMAEEMREMIERQGEQIERIEKKAEEAKKKIEEQNERAERLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 365
SEREKEIEEGLERVSEIVRRLKELAEEMRRMIEEQGRRIDRIEEKADKAKEEIEKQNEKLEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 366
SEREKEIDEGLEKVSEIVKELKELAKEMKEMIEEQGRRIDRIERKAEETKKKIEELNEQAERLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 367
SEREEEIDKGLERVSEIVKKLKELAEKMKEEIERQGEQIDRIEKKADETIKEIERLNESADRLLKSPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 368
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 369
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 370
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 371
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 372
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 373
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 374
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 375
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 376
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVED
> SEQ ID NO: 377
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKESLANKEERHKQILELEEKIKELYEMEKELSEKIEEQLKKIDRIEEKV
SEASRHVSKGVES
> SEQ ID NO: 378
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKTRRTLAEIEERHRQILELEEKIEELYEMFKELSEKISEQGQKISRIEDKV
SKASEHVSKGVEN
SEQ ID NO: 379
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKSLAEIEKRHEQILQLEKQIEELHEMFKELSEKISKQGQKIDRIEEKV
EEAKRHVEKAVKD
> SEQ ID NO: 380
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKKVKEELARIEARHQQILALEEKIRELYEMEKELSEKIEEQGKKIDRIEDKV
SKASEHVSKGVEN
> SEQ ID NO: 381
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKKVKEELKEKEKRHRQIEELLKKIEELHEMFEELSERISEQGQKIDRIDDKV
SKASEHVSKGVED
> SEQ ID NO: 382
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEEVKKSLAEIEKRHEQILALEKKIEELYEMEKELGEKIEKQLQKISRIEEKV
SEASRHVSKGVED
> SEQ ID NO: 383
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEIEARHQQIEALLEQIKELYEMEKELSEKIEEQGQKISRIEDKV
SKASEHVSKGVEQ
> SEQ ID NO: 384
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEKEKRHKQIDELLEKIKELYEMFKEMGEKIEKQGEKIDRIEKKV
SEASKHVSKAVED
> SEQ ID NO: 385
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEAVKKELAAIEARHKQIDALLEKIKELHEMFEEMSKKIEEQMQKISRIEDKV
SEASRHVSKAVSD
> SEQ ID NO: 386
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEIEKRHKQILELEEKIKELHEMFKELGEKIEKQGQKISRIDDKV
SEAKRHVEKGVED
> SEQ ID NO: 387
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKKVKEELKKIEERHKQILELEEKIEELYEMEKELAERIEKQGEKIDRIDEKV
SEAKRNVEKAVED
> SEQ ID NO: 388
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELEEKEKRHKQILELEEKIKELYEMEKELSEKIEEQLQKIDRIDDKV
SEASRHVSKGVED
> SEQ ID NO: 389
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEEVRRSLEEIERRHRQILELEEKIEELYEMEKEMSEKIEEQGQKISRIEEKV
SKASEHVSKAVED
> SEQ ID NO: 390
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEEVKKELAEIEARHEQIKELEKQIEELHEMFKELGEKIEKQGEKIDRIDEKV
SEASRHVSKAVED
> SEQ ID NO: 391
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKEELAKKEARHEQILELTEKIEELSEMFKELSEKISEQGQKIDRIEDKV
SKASEHVSKAVED
> SEQ ID NO: 392
SELEKKIDELLEEISRLVRELKEIAKELRRLTERQGRQVERIEREVEEAEREIEELNKEAEELLEKEDSEDE
LEKLKKLLEESKEQLREVERIEREVRRLREEQRRLLELTERAARLAEEALEKMEKMLELQEKILESMKEPDK
PYTPEELEKVKERHELIKKLKEEIKELKEMFEELRELVRRQGERLDRIEEKVRRAVEHVKKAEEN
> SEQ ID NO: 393
SEKEKEIDELLDKVSEIVKELKKLAEELKRRTERQGRQIEEIERKTEEAKRKIEELNKKAEELLKKEDDDSD
LEKTKELLKEAKEQLREVKEIKRRVEELKREQEETLKLTKEAAELAEEAKELMEEMLELSEEILEEMLENPK
PYTPEELEKVRERHELIKKLLEEIEELEEMFEELERLVEEQGRRLERIEEKVSRAVRHVERAEEN
> SEQ ID NO: 394
SSKEEEIEELLDEVSEIVRRLKEMAREIREMVERQGRQIERIERKVEEAKRKIEELNKKAEELLEKEDDESE
LEELEKLVEEAKEQLREVEEINREVEELGREQERLLRKTREAAKLAEKAEELMKKMLELSEEILEEMKEKPK
EYTPEELEEVEERHKLIQKLLEEIKELKEMFEELERLVEEQGRRLERIEEKVRRAVEHVKRALEN
> SEQ ID NO: 395
SELEKKIDELLEEISRLVRELKEIAKELRRLTERQGRQVERIEREVEEAEREIEELNKEAEELLEKEDSEDE
LEKLKKLLEESKEQLREVERIEREVRRLREEQRRLLELTERAARLAEEALEKMEKMLELQEKILESMKEPDK
PYTPEELEKVKERHELIKKLKEEIKELKEMFEELRELVRRQGERLDRIEEKVRRAVEHVKKAEEN
> SEQ ID NO: 396
SEKEKEIDELLDKVSEIVKELKKLAEELKRRTERQGRQIEEIERKTEEAKRKIEELNKKAEELLKKEDDDSD
LEKTKELLKEAKEQLREVKEIKRRVEELKREQEETLKLTKEAAELAEEAKELMEEMLELSEEILEEMLENPK
PYTPEELEKVRERHELIKKLLEEIEELEEMFEELERLVEEQGRRLERIEEKVSRAVRHVERAEEN
> SEQ ID NO: 397
SETEKKENEALEELERLLEEAKKLLEEQRRLLEAQGEVQKEQEKLEDELEEIQEEAEKYQNKLLESKDEEDE
MSLLKKALELLEKASKLLEELEALLKKQKELLEKQKELMKELEEVLKKIEEKLKKIKELQEEELKEKKKELA
EAEAAEKAGKAAGVSLKEEVEKVKRELAQIEERHQQILELEEKIKELLEMFKELSEKIEEQGQKIDRIEDKV
SKAKEHVEKGVED
> SEQ ID NO: 398
SSLEKKIDENLEKALELLEELKEKLEEMRRLLEESGRLQDELEELMDETQKQQEELEKLLEKLLKMDDSDEQ
YELLKEALKKQKELKEQLEELEEKLKELRRAHEETRRKMEEAEELLKELEEVMEELKKAQEELLKEKKKKYE
EAKKELEEAKKKGEEGKEKLEKVKRELAQIEERHQQILELEEKIKELLEMFKELSEKIEEQGQKIDRIEDKV
SKAKEHVEKGVED
> SEQ ID NO: 399
SEEKKKMEELLDKIKELLEELKKLAEEIKKLLEEQGRQLEKLEEEADKALRQAEEAIRLQEKALELEDDEEI
DEALKELEEKQKKLKEQLEKLEEQISELKKLFEEQKKKMEEAEELLKEMLELIKEMKENHEKLLEEAKKRYE
EKLKEYEELKKLGILPKEELEKVKRELAQIEERHQQILELEEKIKELLEMFKELSEKIEEQGQKIDRIEDKV
SKAKEHVEKGVED
> SEQ ID NO: 400
EEFEKKENELLEELKKKLEEAKKLLKENRRLLEEQGRQLEEIEEKMEEAEELQEKALEYQEKAEKAGFSDES
FEYLKEALKVLEELEEQLEEIEEKLEEQRELLEKQRELLKEAEKKLKEAEEVCKKLKELIEKRKEEAEEKLK
KAEEKAKEAAKKGVDLSEELEKVKRELAQIEERHQQILELEEKIKELLEMFKELSEKIEEQGQKIDRIEDKV
SKAKEHVEKGVED
> SEQ ID NO: 401
SEEEEKFNELLEKIEEELEEIKELAEELREKLEELGRLTEKALELADELEKLFEEAEKLLEEALKLGDGEEL
EEVLKEALEKLKEAKEKLEKLEEELSKLKEAQEEAKELLEELEEELKELEEEIKKLKELSEETLKKAKEKLK
EAEKKAEELKKLGIDPTEELEKVKRELAQIEERHQQILELEEKIKELLEMFKELSEKIEEQGQKIDRIEDKV
SKAKEHVEKGVED
> SEQ ID NO: 402
SSLKEKFNELLDELLKELEEAKELLEEIREQLERIGEQLEELEEQFDEILKEQEELEKQQKKLLESPGSEEE
EEQLKEIEEKQKKIKEKIEELEEQIEELKEQQEKLKELTKELEEKLKEISETLKELKKVQEELLKEKKKKYE
EAKKKYEEKKKKGINGTEELEKVKRELAQIEERHQQILELEEKIKELLEMFKELSEKIEEQGQKIDRIEDKV
SKAKEHVEKGVED
> SEQ ID NO: 403
KESEEKFEEKLKELEKLLEEAKELLEKQREYLEESGRLLDEAEKLMDETERIFEETLKLQDKLLAAKDEEDQ
MSLLKKALELLEKASKLLDELEATLKELKALLEKQKELMEELEKVLKEIEEKMKEIKKLQEEELKEQKKKLK
EAEEKEKEGEKKGVSYKEEVEKVKRELAQIEERHQQILELEEKIKELLEMFKELSEKIEEQGQKIDRIEDKV
SKAKEHVEKGVED
> SEQ ID NO: 404
SKLKEEFEKYLEELLKQLEKLKEKLKELREKLEEQGKQLEKLEEQFDRILEQQEKLLEQQEKLLEDEGSEEE
EELLKEIEKQQEKLKEEIEKLEEQIKKLKEQQEELKEISEKAKELLKKTAEILKKLKEVQEKLLEEKKKELE
EAEKKYEELKKKGINGTEELEKVKRELAQIEERHQQILELEEKIKELLEMFKELSEKIEEQGQKIDRIEDKV
SKAKEHVEKGVED
> SEQ ID NO: 405
SEREKEFNERLEEMEKLLEKIKKLAEEIRRLLEKQGELLDKLEELADEALRLQEKAIEKSEKILEKGYNEET
EEELKELLKLLKELEELLDEAEELIDEIKRLLEEQKKLMEEMEKALKKLEELTKKLKELIEKELEEQRKRLE
ELEKRKEEYEKLGIDLSEELEKVKRELAQIEERHQQILELEEKIKELLEMFKELSEKIEEQGQKIDRIEDKV
SKAKEHVEKGVED
> SEQ ID NO: 406
SSFEEEINKLLEELKRKLEELKEILEEIRKLLEEQGRQLDEIEEKMDEAEELAEKAEEYLKKAEEAGGGEES
YEYLKKALETLKELEEKLDEIEEKLSEQKKLLEETREKLEEAEKKLKEAEKVIKKLKELIEKEKKEKEAELK
KAEAAAAEAAKLGIDKSEELEKVKRELAQIEERHQQILELEEKIKELLEMFKELSEKIEEQGQKIDRIEDKV
SKAKEHVEKGVED
> SEQ ID NO: 407
SEEEEKFNELLEKIEEKLEEAKELAEELREELEKIGELTDEAERLADEALKLAEEAEKLLKEALKLGDEDEL
DKILKEAEKTLEELKKKLEELEEKLKELKEAQEKAKELLKELEETLKELEELIKELKKFSEETLEKAKEKYK
KAEEKYKEDLKKGIDNTEEIEKVKRELAQIEERHQQILELEEKIKELLEMFKELSEKIEEQGQKIDRIEDKV
SKAKEHVEKGVED
> SEQ ID NO: 408
ESFEKELEELLEKIQELMEKIKELAEKLREALEESGRLLEEIEEAVDKLEEKFEEIEKLQENAEKYEDTEEA
EKYLKEMEEKLKKAKELLDKLEELVSKLKELQEKQRELMEKLEEKLKELLELLKKLKELIEKLKEKKKKELE
EAEKKLKEAEEYNEELEKEVEKVKRELAQIEERHQQILELEEKIKELLEMFKELSEKIEEQGQKIDRIEDKV
SKAKEHVEKGVED
> SEQ ID NO: 409
SELEEKENKYLEELLETLEKLKEALEKIREKLEEQGKQLDKIEEAFDELLKQQEELLKQQEELLADPGSEES
EKKLKEIEKQQEKIKEQIEKLEEQIKKLRELQEKQKELTEKAKELLEKLEEILKKLKEVQEKLLEEKKKEYE
EAEKEYKEDKKKGINNKEKLEKVKRELAQIEERHQQILELEEKIKELLEMFKELSEKIEEQGQKIDRIEDKV
SKAKEHVEKGVED
> SEQ ID NO: 410
SSLEKKIDENLEKALELLEELKEKLEEMRRLLEESGRLQDELEELMDETQKQQEELEKLLEKLLKMDDSDEQ
YELLKEALKKQKELKEQLEELEEKLKELRRAHEETRRKMEEAEELLKELEEVMEELKKAQEELLKEKKKKYE
EAKKELEEAKKKGEEGKEKLEKVKRELAQIEERHQQILELEEKIKELLEMFKELSEKIEEQGQKIDRIEDKV
SKAKEHVEKGVED
> SEQ ID NO: 411
SSLEKKIDENLEKALELLEELKEKLEEMRRLLEESGRLQDELEELMDETQKQQEELEKLLEKLLKMDDSDEQ
YELLKEALKKQKELKEQLEELEEKLKELRRAHEETRRKMEEAEELLKELEEVMEELKKAQEELLKEKKKKYE
EAKKELEEAKKKGEEGKEKLEKVKRELAQIEERHQQILELEEKIKELLEMFKELSEKIEEQGQKIDRIEDKV
SKAKEHVEKGVED
> SEQ ID NO: 412
GELKEKKEKLSKEFEKLLKESKRLAEELKEKLEELGRALDEAEELADEVERQQEELEKLQEEILKSEENEDE
KKQLEELEKKLKELEELLKELEEKLKEVEELMKEVEELMEELEKTMEEMEKAIEELEKVYKEELKKTEAKLK
ATKAEAEAAKAKGEDISDKLEAAEKEYKSVKEELKLVEEIKKKVEEIKEMLEEMKERIEEMEEKVKRIEEKL
KRIEESLKRVEEN
> SEQ ID NO: 413
DELEKKIKELEEKSEEELKEAKELAEELRRLLEELERALDEAERLADEVERKQEELEKLMEEMLKSEDNESD
EEDLKKLKEKLEELEKLLEELEERAREVEELMERVEETMEELEEEMEELLETLKKLLEVYEELLKKKKKELE
ETKKKAEEMKKKGIDISEELEKAKEELESVKKNLELVKKILEEVKEIKEELEEMGEEIERMEEKVDRIEEKL
ERVEESLERVSKN
> SEQ ID NO: 414
SEEDKKMEELLEEALKLLEELKELLEKNRELLEELGRQQEELEKLQDEAERLQEELEEAFKKMEENEESEEG
KKYLEEAEKLLKELKKLLEEIEKKTKEIEELVKKQEELMKKIKEVMKKLEEKMKELYRISKERLERAKEEAA
RAEAARAEYEAAGSPEVERAEQVLEEYREAKEFYEKVEELLREVKEIKEEIKEMEERIKEIGERIKRIEEKI
ERVEKLLERTEKN
> SEQ ID NO: 415
SEKEKEFNELLEEALRELEKLKELLEENGRLLERTGEQLERMEELMDEAEEKQEELEEAIKKMEKYEDSEEG
DEYLEEAEELLEELEELLEEIEAQTEEIEALIKEQEELMKKIKEEMEKLKEAVEKLYEISKEMLEEAKKEYE
KAEKAKAEYEAAGKDEVKECEKVKEKYEEAKKRYEQVEKLLKEVEEIKEEIERMGEEIKRQGERIERIEEKI
ERVEEELERLEEN

TABLE 13

| # | amino aciduence |
|---|---|
| N9 | KPGEEKLNKLLEELLKKLEELKKLAEENRRLLERQGRQLEELERRFEELNRRMEELNEKLEKLLKEEPNEE |
| | TG EKLEEIKKELEELSRELKELEERVRRQEEEHERQREVVEEIKKELEEAKKYCEELLKTSEEILEEMLEN |
| | PKPYTPEELEKVRERHELIKKLLEEIEELEEMFEELERLVEEQGRRLERIEEKVSRAVRHVERAEEN |
| | SEQ ID NO: 416 |
| N10 | EPKEKELEELLEELLRELEEIKKLLEEFRRLQEEIGRQIEEIERQLEELLERLEELNEKLENLLKREDNEN |
| | DLEELKELLEEMRELGREMRELERRVEELGRLLEEQRRLVEELKKKLERLLELVKRLLELVEEILEEMLEN |
| | PKPYTPEELEKVRERHELIKKLLEEIEELEEMFEELERLVEEQGRRLERIEEKVSRAVRHVERAEEN |
| | SEQ ID NO: 417 |
| N11 | RPEEEKLNELLDELLRLLEEIKKLLEENRALLEEIGRQIDRIEEQLDRLLRELKELNEKLEALLKREDNEN |
| | DLEELKELLEEIKRLSEEMKELEREVERLGELLEEQRRKVEELKRKLEELLELTEEALELVEEILEEMLEN |
| | PKPYTPEELEKVRERHELIKKLLEEIEELEEMFEELERLVEEQGRRLERIEEKVSRAVRHVERAEEN |
| | SEQ ID NO: 418 |
| N12 | SLEEILEKLKEIAELLEEVEELTEELKEETERAGRELEELERRLEELVRRAEELNRKLEKILEEEDSDDIL |
| | ERLKEARRELRELRERLEEVEREIERLIREAEEQSELLEELERELEEIKELLKELLEKEEELSEEELELIK |
| | KLLEEIEELEEMFEELERLVEEQGRRLERIEEKVSRAVRHVERAEEN SEQ ID NO: 419 |
| N28 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVFRRISGGSGGSGGSGGSDARENEMDENLEQVSGIIGNLRHMALDMGNEIDT |
| | QNRQIDRIMEKADSAKTRIDEANQRATKMLGSGGGSGGSEELKKLEKEGEKLKELVEELDREIKELKE |
| | GMERLREMFEEAAKLSEEALEIMRRTRKLSEEELEEAKQKAKEQEKEKALKEKGGSGGSGGSEKVKRE |
| | LAQIEERHQQILELEEKIKELLEMFKELSEKIEEQGQKIDRIEDKVSKAKEHVEKGVED |
| | SEQ ID NO: 420 |
| N29 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVFRRISGGSGGSGGSGGSGGSGGSGGSGGSDARENEMDENLEQVSGIIGNLRH |
| | MALDMGNEIDTQNRQIDRIMEKADSAKTRIDEANQRATKMLGSGGGSGGSEELKKLEKEGEKLKELVE |
| | ELDREIKELKEGMERLREMFEEAAKLSEEALEIMRRTRKLSEEELEEAKQKAKEQEKEKALKEKGGSG |
| | GSGGSEKVKRELAQIEERHQQILELEEKIKELLEMFKELSEKIEEQGQKIDRIEDKVSKAKEHVEKGV |
| | ED SEQ ID NO: 421 |
| N30 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVFRRISGGSGGSGGSGGSSEREKEIDEGLDRVSEIVKELKKMAEEMRRMIEE |
| | QGRRIERIEEKAEEAKEKIEEANERAEKLLKDPGGSGGSEELKKLEKEGEKLKELVEELDREIKELKE |
| | GMERLREMFEEAAKLSEEALEIMRRTRKLSEEELEEAKQKAKEQEKEKALKEKGGSGGSGGSEKVKRE |
| | LAQIEERHQQILELEEKIKELLEMFKELSEKIEEQGQKIDRIEDKVSKAKEHVEKGVED |
| | SEQ ID NO: 422 |
| N31 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSGGSGGSGGSGGSGGSGGSSEREKEIDEGLDRVSEIVKELKK |
| | MAEEMRRMIEEQGRRIERIEEKAEEAKEKIEEANERAEKLLKDPGGSGGSEELKKLEKEGEKLKELVE |
| | ELDREIKELKEGMERLREMFEEAAKLSEEALEIMRRTRKLSEEELEEAKQKAKEQEKEKALKEKGGSG |
| | GSGGSEKVKRELAQIEERHQQILELEEKIKELLEMFKELSEKIEEQGQKIDRIEDKVSKAKEHVEKGV |
| | ED SEQ ID NO: 423 |
| N32 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVFRRISGGSGGSSEREKEIDEGLDRVSEIVKELKKMAEEMRRMIEEQGRRIE |
| | RIEEKAEEAKEKIEEANERAEKLLKDPGGSGGSEELKKLEKEGEKLKELVEELDREIKELKEGMERLR |
| | EMFEEAAKLSEEALEIMRRTRKLSEEELEEAKQKAKEQEKEKALKEKGGSGGSGGSEKVKRELAQIEE |
| | RHQQILELEEKIKELLEMFKELSEKIEEQGQKIDRIEDKVSKAKEHVEKGVED SEQ ID NO: 424 |
| N33 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSSEKEKEIDELLDKVSEIVKELKKLAEELKRRTERQGRQIE |
| | EIERKTEEAKRKIEELNKKAEELLKKEDDDSDLEKTKELLKEAKEQLREVKEIKRRVEELKREQEETL |
| | KLTKEAAELAEEAKELMEEMLELSEEILEEMLENPKPYTPEELEKVRERHELIKKLLEEIEELEEMFE |
| | ELERLVEEQGRRLERIEEKVSRAVRHVERAEEN SEQ ID NO: 425 |

In a further embodiment of this fourth aspect, the X2 JMD domain comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:331-332 and 426-445. The amino acid sequences of SEQ ID NO: 426-445 are shown in Table 14.

TABLE 14
TKKAVKYQSRRRRRR SEQ ID NO: 426
TKKAVKYQSKKKKKK SEQ ID NO: 427
LKEAVEYDEKARRKK SEQ ID NO: 428
LKKAVEYDEKARRKK SEQ ID NO: 429
LKEAVEYEEKARRKK SEQ ID NO: 430
LKEAVKYREESEKME SEQ ID NO: 431
LKEAVKYNEEGKKME SEQ ID NO: 432
LKEAVEYREKSEKME SEQ ID NO: 433
LKEAVKYKEESEKME SEQ ID NO: 434
LKKAVEYKEKSEKKE SEQ ID NO: 435
TKKAVKYQSESEKME SEQ ID NO: 436
TKKAVKYQSEAEKME SEQ ID NO: 437
TKKAVKYQSEAEKKE SEQ ID NO: 438
IGEAVKYLEKSKELE SEQ ID NO: 439
LGEAVEYLEKSKKLE SEQ ID NO: 440
LKEAKEYRKKNEELE SEQ ID NO: 441
LKEIKELAKKREEKG SEQ ID NO: 442
LEEIERLFEERKEKG SEQ ID NO: 443
LKEIKELRDKIEKNG SEQ ID NO: 444
LEEIKKLREKIKENG SEQ ID NO: 445

In another embodiment, the X3 TMD domain comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:223-234. The amino acid sequences of the X3 TMDs is provided in Table 4.

In further embodiments of this fourth aspect, the nucleic acid encodes a polypeptide comprising the formula B1-B2-X1-X2-X3, wherein
B1 comprises the amino acid sequence at least 90%, 91%, 92%, 93%, 94%, 95%, 96%, 97%, 98%, 99%, or 100% of SEQ ID NO:290 or 291; and
B2 comprises an optional amino acid linker, which may be present or absent.

In these embodiments the fusion protein can be used for inducible binding to v-SPs (described above) in the presence of rapamycin. The sc-t-SPs can be fused to the FRB domain (SEQ ID NO:291) at the N-terminus and FRB can bind to its cognate binding partner FKBP domain (e.g. SEQ ID NO:290) fused to the N-terminus of v-SPs (e.g. SEQ ID NO 148) only in the presence of rapamycin. The fusion activity of designed fusogens fused to FKB and FRB can be induced in the presence of rapamycin. FRB domain fused to sc-t-SPs and FKBP domain fused to v-SPs would function similarly if they were interchanged with each other.

In one embodiment, the nucleic acids of this fourth aspect encode a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:64-146, 148, and 446-455

The amino acid sequence of SEQ ID NO:64-146 and 446-455 are shown in Tables 15 and 16.

In a further embodiment, the nucleic acids of this fourth aspect encode a polypeptide that further comprises a signal peptide at its amino-terminus. Any signal peptide may be used as suitable for an intended purpose. The signal peptide may be directly linked to the polypeptide, or may be connected via an amino acid linker. In some embodiments, the signal peptide comprises the amino acid sequence selected from the group consisting of SEQ ID NO:292-309. The amino acid sequence of these exemplary signal peptides are provided in Table 7.

In a further embodiment, the nucleic acids of the second aspect comprise an expression vector comprising the nucleic acid operatively linked to a control sequence, such as a promoter.

TABLE 15
>SEQ ID NO: 64
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEEEEEGGSGGSGGSSISKQALSEIETRHSEIIKLENSIRELHDMFMDMAMLVESQGEMIDRIEYNV
EHAVDYVERAVSDTKKAVKYQSKARRKKIMIIICCVILGIVIASTVGGIFA
> SEQ ID NO: 65
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSDTKKAVKYQSKARRKKIMIIICCVILGIVIASTVGGIFA
> SEQ ID NO: 66
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEGGSGGSGGSSISKQALSEIETRHSEIIKLENSIRELHDMEMDMAMLVESQGEMIDRIEYNVEHAVD
YVERAVSDTKKAVKYQSKARRKKIMIIICCVILGIVIASTVGGIFA
> SEQ ID NO: 67
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EGGSGGSGGSSISKQALSEIETRHSEIIKLENSIRELHDMEMDMAMLVESQGEMIDRIEYNVEHAVDYVERA
VSDTKKAVKYQSKARRKKIMIIICCVILGIVIASTVGGIFA
> SEQ ID NO: 68
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKGGSG
GSGGSSISKQALSEIETRHSEIIKLENSIRELHDMEMDMAMLVESQGEMIDRIEYNVEHAVDYVERAVSDTK
KAVKYQSKARRKKIMIIICCVILGIVIASTVGGIFA
> SEQ ID NO: 69
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELGGSGGSGGS
SISKQALSEIETRHSEIIKLENSIRELHDMEMDMAMLVESQGEMIDRIEYNVEHAVDYVERAVSDTKKAVKY
QSKARRKKIMIIICCVILGIVIASTVGGIFA
> SEQ ID NO: 70
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKGGSGGSGGSSISKQ
ALSEIETRHSEIIKLENSIRELHDMEMDMAMLVESQGEMIDRIEYNVEHAVDYVERAVSDTKKAVKYQSKAR
RKKIMIIICCVILGIVIASTVGGIFA
> SEQ ID NO: 71
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EEEEEEGGSGGSGGSALSEIETRHSEIIKLENSIRELHDMFMDMAMLVESQGEMIDRIEYNVEHAVDYVERA
VSDTKKAVKYQSKARRKKIMIIICCVILGIVIASTVGGIFA
> SEQ ID NO: 72
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKEEEE
EGGSGGSGGSALSEIETRHSEIIKLENSIRELHDMEMDMAMLVESQGEMIDRIEYNVEHAVDYVERAVSDTK
KAVKYQSKARRKKIMIIICCVILGIVIASTVGGIFA
> SEQ ID NO: 73
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELLKEAKGGSG
GSGGSALSEIETRHSEIIKLENSIRELHDMFMDMAMLVESQGEMIDRIEYNVEHAVDYVERAVSDTKKAVKY
QSKARRKKIMIIICCVILGIVIASTVGGIFA
> SEQ ID NO: 74
DARENEMDENLEQVSGIIGNLRHMALDMGNEIDTQNRQIDRIMEKADSAKTRIDEANQRATKMLGSGGGSGG
SEELKDLEKEGKELKELVEELDREVKELKESMEKLKEMTEEAAELSSQALEIMRRTRKLSEELGGSGGSGGS
ALSEIETRHSEIIKLENSIRELHDMEMDMAMLVESQGEMIDRIEYNVEHAVDYVERAVSDTKKAVKYQSKAR
RKKIMIIICCVILGIVIASTVGGIFA
> SEQ ID NO: 75
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKEELAKVEERHKQIQALLDKIEELYEMFKEMSEKISEQGQKIDRIEEKV
SKASEHVSKGVEDTKKAVKYQSKARRKKIMIIICCVILGIVIASTVGGIFA
> SEQ ID NO: 76
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEVEKRHKQILELEEKIKELYEMEKEMSEKIEKQGQKIDRIDDKV
SEAKKHVEKAVEDTKKAVKYQSKARRKKIMIIICCVILGIVIASTVGGIFA
> SEQ ID NO: 77
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 78
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEAVKKSLAAKEERHKQILELLEKIKELHEMFKELSEKIEKQGQKIDRIEDKV
SKASEHVSKGVEDTKKAVKYQSKARRKKIMIIICCVILGIVIASTVGGIFA
> SEQ ID NO: 79
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEAVKKELAAIEERHEQILELLKKIEELYEMEKELSEKIEKQGQKIDRIEKKV
SEASRHVSKAVEDTKKAVKYQSKARRKKIMIIICCVILGIVIASTVGGIFA
> SEQ ID NO: 80
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKDLAAIEERHQQILELEEKIKELHEMFKEMSEKISEQMQKIDRIEEKV
SKASEHVSKGVEDTKKAVKYQSKARRKKIMIIICCVILGIVIASTVGGIFA
> SEQ ID NO: 81
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVRRELAAIEERHRQILELLEKIEELHEMFKEMSEKISKQMEKIDRIDDRV
SEASRHVEKGVEDTKKAVKYQSKARRKKIMIIICCVILGIVIASTVGGIFA
> SEQ ID NO: 82
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKAVKEELANIENRHKQIDALYEKIKELHEMFLEMSERIEAQLQKIDRIDDKV
SKAKAHVEKGVEDTKKAVKYQSKARRKKIMIIICCVILGIVIASTVGGIFA
> SEQ ID NO: 83
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSDTKKAVKYQSKARRKKIMIIICCVILGIVIASTVGGIFA
> SEQ ID NO: 84
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSALSEIETRHSEIIKLENSIRELHDMEMDMAMLVESQGEMIDRIEYNVEHAVD
YVERAVSDTKKAVKYQSKARRKKIMIIICCVILGIVIASTVGGIFA
> SEQ ID NO: 85
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSDTKKAVKYQSKARRKKLLLLLLLLLLLLLLLLLLLLLFA
> SEQ ID NO: 86
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSDTKKAVKYQSKARRKKMMIILGVICAIILIIIIVYFFA
> SEQ ID NO: 87
NKREEEIDKGLDRVGEIISKLNEMAREMGEKIEEQNQKISEIEKKADEAIEKVEKLIKDAEKLLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSSISKQALSEIETRHSEIIKLENSIRELHDMEMDMAMLVESQGEMIDRIEYNV
EHAVDYVERAVSDTKKAVKYQSRRRRRRMMIILGVICAIILIIIIVYFFA
> SEQ ID NO: 88
NEREKEIDEGLERVGELISKLKELAREMSEKIEEQNQKLSEIDKKAEEAIKLLEKANASAKKLLEKPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 89
NEREKEIEEGLERVGELISELKEMAREMSEKIEEQNKKLDEISKKADEAIKLLEKANKGAEELLKKPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 90
NEREKEIDEGLEKIGELISKLKEMAREMSEKIEEQNEKLDEIDKKADEAIKLLEEANKKAEKLLKKKGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 91
NEREKEIEEGLERIGELISKLKELAREMSEKIEEQNEKLSEISEKADEAIKLLEKANASAQKLLEKPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 92
NPREEEIDKGLEEIGKLISELKELAREMSEKIEEQNEKISEIDEKAKEAIELLKKANEKAKELLEKEGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 93
SPREKEIDEGLERVSELVKKLKELAEKMKEMIEEQGRRIERIERKAEEAKERIEKLNEKAEKLLEDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 94
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 95
SEREKEIDEGLEKVSEIVKELKEMAEEMREMIERQGEQIERIEKKAEEAKKKIEEQNERAERLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 96
SEREKEIEEGLERVSEIVRRLKELAEEMRRMIEEQGRRIDRIEEKADKAKEEIEKQNEKLEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 97
SEREKEIDEGLEKVSEIVKELKELAKEMKEMIEEQGRRIDRIERKAEETKKKIEELNEQAERLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 98
SEREEEIDKGLERVSEIVKKLKELAEKMKEEIERQGEQIDRIEKKADETIKEIERLNESADRLLKSPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 99
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSRRRRRRIMIIICCVILGIVIASTVGGIFA
> SEQ ID NO: 100
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKKKKKKIMIIICCVILGIVIASTVGGIFA
> SEQ ID NO: 101
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKAAVLVLLVIVIISLIVLVVIW
> SEQ ID NO: 102
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKMMVVVVVVVVVVVVVVVVYF
> SEQ ID NO: 103
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKARRKKMMIIIIIIIIIIIIIIIIYF
> SEQ ID NO: 104
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSRRRRRRAAVLVLLVIVIISLIVLVVIW
> SEQ ID NO: 105
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSRRRRRRMMVVVVVVVVVVVVVVVVYF
> SEQ ID NO: 106
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKKKKKKAAVLVLLVIVIISLIVLVVIW
> SEQ ID NO: 107
SEREKEIDEGLDRVSEIVKELKKMAEEMRRMIEEQGRRIERIEEKAEEAKEKIEEANERAEKLLKDPGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKRELAQIEERHQQILELEEKIKELLEMFKELSEKIEEQGQKIDRIEDKV
SKAKEHVEKGVEDTKKAVKYQSKKKKKKMMVVVVVVVVVVVVVVVVYF
> SEQ ID NO: 108
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKESLANKEERHKQILELEEKIKELYEMFKELSEKIEEQLKKIDRIEEKV
SEASRHVSKGVESLKEAVEYDEKARRKKIMIIICCVILGIVIASTVGGIFA
> SEQ ID NO: 109
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKTRRTLAEIEERHRQILELEEKIEELYEMEKELSEKISEQGQKISRIEDKV
SKASEHVSKGVENLKKAVEYDEKARRKKIMIIICCVILGIVIASTVGGIFA
> SEQ ID NO: 110
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKSLAEIEKRHEQILQLEKQIEELHEMEKELSEKISKQGQKIDRIEEKV
EEAKRHVEKAVKDLKEAVEYEEKARRKKIMIIICCVILGIVIASTVGGIFA
> SEQ ID NO: 111
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKKVKEELARIEARHQQILALEEKIRELYEMFKELSEKIEEQGKKIDRIEDKV
SKASEHVSKGVENLKEAVEYDEKARRKKIMIIICCVILGIVIASTVGGIFA
> SEQ ID NO: 112
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKKVKEELKEKEKRHRQIEELLKKIEELHEMFEELSERISEQGQKIDRIDDKV
SKASEHVSKGVEDLKEAVEYEEKARRKKIMIIICCVILGIVIASTVGGIFA
> SEQ ID NO: 113
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEEVKKSLAEIEKRHEQILALEKKIEELYEMEKELGEKIEKQLQKISRIEEKV
SEASRHVSKGVEDLKEAVKYREESEKMEIMIIICCVILGIVIASTVGGIFA
> SEQ ID NO: 114
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEIEARHQQIEALLEQIKELYEMFKELSEKIEEQGQKISRIEDKV
SKASEHVSKGVEQLKEAVKYNEEGKKMEIMIIICCVILGIVIASTVGGIFA
> SEQ ID NO: 115
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEKEKRHKQIDELLEKIKELYEMFKEMGEKIEKQGEKIDRIEKKV
SEASKHVSKAVEDLKEAVEYREKSEKMEIMIIICCVILGIVIASTVGGIFA
> SEQ ID NO: 116
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEAVKKELAAIEARHKQIDALLEKIKELHEMFEEMSKKIEEQMQKISRIEDKV
SEASRHVSKAVSDLKEAVKYKEESEKMEIMIIICCVILGIVIASTVGGIFA
> SEQ ID NO: 117
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELAEIEKRHKQILELEEKIKELHEMFKELGEKIEKQGQKISRIDDKV
SEAKRHVEKGVEDLKKAVEYKEKSEKKEIMIIICCVILGIVIASTVGGIFA
> SEQ ID NO: 118
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSKKVKEELKKIEERHKQILELEEKIEELYEMEKELAERIEKQGEKIDRIDEKV
SEAKRNVEKAVEDTKKAVKYQSESEKMEIMIIICCVILGIVIASTVGGIFA
> SEQ ID NO: 119
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKKELEEKEKRHKQILELEEKIKELYEMEKELSEKIEEQLQKIDRIDDKV
SEASRHVSKGVEDTKKAVKYQSEAEKMEIMIIICCVILGIVIASTVGGIFA
> SEQ ID NO: 120
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEEVRRSLEEIERRHRQILELEEKIEELYEMFKEMSEKIEEQGQKISRIEEKV
SKASEHVSKAVEDTKKAVKYQSEAEKKEIMIIICCVILGIVIASTVGGIFA
> SEQ ID NO: 121
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEEVKKELAEIEARHEQIKELEKQIEELHEMFKELGEKIEKQGEKIDRIDEKV
SEASRHVSKAVEDTKKAVKYQSESEKMEIMIIICCVILGIVIASTVGGIFA
> SEQ ID NO: 122
DARENEMDENLEQVSGIIGNLRHMALDMGNEIDTQNRQIDRIMEKADSAKTRIDEANQRATKMLGSGGGSGG
SEELKKLEKEGEKLKELVEELDREIKELKEGMERLREMFEEAAKLSEEALEIMRRTRKLSEEELEEAKQKAK
EQEKEKALKEKGGSGGSGGSEKVKEELAKKEARHEQILELTEKIEELSEMFKELSEKISEQGQKIDRIEDKV
SKASEHVSKAVEDTKKAVKYQSESEKMEIMIIICCVILGIVIASTVGGIFA
> SEQ ID NO: 123
SELEKKIDELLEEISRLVRELKEIAKELRRLTERQGRQVERIEREVEEAEREIEELNKEAEELLEKEDSEDE
LEKLKKLLEESKEQLREVERIEREVRRLREEQRRLLELTERAARLAEEALEKMEKMLELQEKILESMKEPDK
PYTPEELEKVKERHELIKKLKEEIKELKEMFEELRELVRRQGERLDRIEEKVRRAVEHVKKAEENIGEAVKY
LEKSKELEIMIIICCVILGIVIASTVGGIFA
> SEQ ID NO: 124
SEKEKEIDELLDKVSEIVKELKKLAEELKRRTERQGRQIEEIERKTEEAKRKIEELNKKAEELLKKEDDDSD
LEKTKELLKEAKEQLREVKEIKRRVEELKREQEETLKLTKEAAELAEEAKELMEEMLELSEEILEEMLENPK
PYTPEELEKVRERHELIKKLLEEIEELEEMFEELERLVEEQGRRLERIEEKVSRAVRHVERAEENLGEAVEY
LEKSKKLEIMIIICCVILGIVIASTVGGIFA
> SEQ ID NO: 125
SSKEEEIEELLDEVSEIVRRLKEMAREIREMVERQGRQIERIERKVEEAKRKIEELNKKAEELLEKEDDESE
LEELEKLVEEAKEQLREVEEINREVEELGREQERLLRKTREAAKLAEKAEELMKKMLELSEEILEEMKEKPK
EYTPEELEEVEERHKLIQKLLEEIKELKEMFEELERLVEEQGRRLERIEEKVRRAVEHVKRALENLKEAKEY
RKKNEELEIMIIICCVILGIVIASTVGGIFA
> SEQ ID NO: 126
SELEKKIDELLEEISRLVRELKEIAKELRRLTERQGRQVERIEREVEEAEREIEELNKEAEELLEKEDSEDE
LEKLKKLLEESKEQLREVERIEREVRRLREEQRRLLELTERAARLAEEALEKMEKMLELQEKILESMKEPDK
PYTPEELEKVKERHELIKKLKEEIKELKEMFEELRELVRRQGERLDRIEEKVRRAVEHVKKAEENIGEAVKY
LEKSKELEMMVVVVVVVVVVVVVVVVYF
> SEQ ID NO: 127
SEKEKEIDELLDKVSEIVKELKKLAEELKRRTERQGRQIEEIERKTEEAKRKIEELNKKAEELLKKEDDDSD
LEKTKELLKEAKEQLREVKEIKRRVEELKREQEETLKLTKEAAELAEEAKELMEEMLELSEEILEEMLENPK
PYTPEELEKVRERHELIKKLLEEIEELEEMFEELERLVEEQGRRLERIEEKVSRAVRHVERAEENLGEAVEY
LEKSKKLEMMVVVVVVVVVVVVVVVVYF
> SEQ ID NO: 128
SETEKKENEALEELERLLEEAKKLLEEQRRLLEAQGEVQKEQEKLEDELEEIQEEAEKYQNKLLESKDEEDE
MSLLKKALELLEKASKLLEELEALLKKQKELLEKQKELMKELEEVLKKIEEKLKKIKELQEEELKEKKKELA
EAEAAEKAGKAAGVSLKEEVEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 129
SSLEKKIDENLEKALELLEELKEKLEEMRRLLEESGRLQDELEELMDETQKQQEELEKLLEKLLKMDDSDEQ
YELLKEALKKQKELKEQLEELEEKLKELRRAHEETRRKMEEAEELLKELEEVMEELKKAQEELLKEKKKKYE
EAKKELEEAKKKGEEGKEKLEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 130
SEEKKKMEELLDKIKELLEELKKLAEEIKKLLEEQGRQLEKLEEEADKALRQAEEAIRLQEKALELEDDEEI
DEALKELEEKQKKLKEQLEKLEEQISELKKLFEEQKKKMEEAEELLKEMLELIKEMKENHEKLLEEAKKRYE
EKLKEYEELKKLGILPKEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 131
EEFEKKENELLEELKKKLEEAKKLLKENRRLLEEQGRQLEEIEEKMEEAEELQEKALEYQEKAEKAGFSDES
FEYLKEALKVLEELEEQLEEIEEKLEEQRELLEKQRELLKEAEKKLKEAEEVCKKLKELIEKRKEEAEEKLK
KAEEKAKEAAKKGVDLSEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 132
SEEEEKFNELLEKIEEELEEIKELAEELREKLEELGRLTEKALELADELEKLFEEAEKLLEEALKLGDGEEL
EEVLKEALEKLKEAKEKLEKLEEELSKLKEAQEEAKELLEELEEELKELEEEIKKLKELSEETLKKAKEKLK
EAEKKAEELKKLGIDPTEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 133
SSLKEKFNELLDELLKELEEAKELLEEIREQLERIGEQLEELEEQFDEILKEQEELEKQQKKLLESPGSEEE
EEQLKEIEEKQKKIKEKIEELEEQIEELKEQQEKLKELTKELEEKLKEISETLKELKKVQEELLKEKKKKYE
EAKKKYEEKKKKGINGTEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 134
KESEEKFEEKLKELEKLLEEAKELLEKQREYLEESGRLLDEAEKLMDETERIFEETLKLQDKLLAAKDEEDQ
MSLLKKALELLEKASKLLDELEATLKELKALLEKQKELMEELEKVLKEIEEKMKEIKKLQEEELKEQKKKLK
EAEEKEKEGEKKGVSYKEEVEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 135
SKLKEEFEKYLEELLKQLEKLKEKLKELREKLEEQGKQLEKLEEQFDRILEQQEKLLEQQEKLLEDEGSEEE
EELLKEIEKQQEKLKEEIEKLEEQIKKLKEQQEELKEISEKAKELLKKTAEILKKLKEVQEKLLEEKKKELE
EAEKKYEELKKKGINGTEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 136
SEREKEFNERLEEMEKLLEKIKKLAEEIRRLLEKQGELLDKLEELADEALRLQEKAIEKSEKILEKGYNEET
EEELKELLKLLKELEELLDEAEELIDEIKRLLEEQKKLMEEMEKALKKLEELTKKLKELIEKELEEQRKRLE
ELEKRKEEYEKLGIDLSEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 137
SSFEEEINKLLEELKRKLEELKEILEEIRKLLEEQGRQLDEIEEKMDEAEELAEKAEEYLKKAEEAGGGEES
YEYLKKALETLKELEEKLDEIEEKLSEQKKLLEETREKLEEAEKKLKEAEKVIKKLKELIEKEKKEKEAELK
KAEAAAAEAAKLGIDKSEELEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 138
SEEEEKFNELLEKIEEKLEEAKELAEELREELEKIGELTDEAERLADEALKLAEEAEKLLKEALKLGDEDEL
DKILKEAEKTLEELKKKLEELEEKLKELKEAQEKAKELLKELEETLKELEELIKELKKESEETLEKAKEKYK
KAEEKYKEDLKKGIDNTEEIEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 139
ESFEKELEELLEKIQELMEKIKELAEKLREALEESGRLLEEIEEAVDKLEEKFEEIEKLQENAEKYEDTEEA
EKYLKEMEEKLKKAKELLDKLEELVSKLKELQEKQRELMEKLEEKLKELLELLKKLKELIEKLKEKKKKELE
EAEKKLKEAEEYNEELEKEVEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 140
SELEEKFNKYLEELLETLEKLKEALEKIREKLEEQGKQLDKIEEAFDELLKQQEELLKQQEELLADPGSEES
EKKLKEIEKQQEKIKEQIEKLEEQIKKLRELQEKQKELTEKAKELLEKLEEILKKLKEVQEKLLEEKKKEYE
EAEKEYKEDKKKGINNKEKLEKVKRELAQIEERHQQILELEEKIKELLEMFKELSEKIEEQGQKIDRIEDKV
SKAKEHVEKGVEDTKKAVKYQSKARRKKIMIIICCVILGIVIASTVGGIFA
> SEQ ID NO: 141
SSLEKKIDENLEKALELLEELKEKLEEMRRLLEESGRLQDELEELMDETQKQQEELEKLLEKLLKMDDSDEQ
YELLKEALKKQKELKEQLEELEEKLKELRRAHEETRRKMEEAEELLKELEEVMEELKKAQEELLKEKKKKYE
EAKKELEEAKKKGEEGKEKLEKVKRELAQIEERHQQILELEEKIKELLEMFKELSEKIEEQGQKIDRIEDKV
SKAKEHVEKGVEDTKKAVKYQSKARRKKAAVLVLLVIVIISLIVLVVIW
> SEQ ID NO: 142
SSLEKKIDENLEKALELLEELKEKLEEMRRLLEESGRLQDELEELMDETQKQQEELEKLLEKLLKMDDSDEQ
YELLKEALKKQKELKEQLEELEEKLKELRRAHEETRRKMEEAEELLKELEEVMEELKKAQEELLKEKKKKYE
EAKKELEEAKKKGEEGKEKLEKVKRELAQIEERHQQILELEEKIKELLEMFKELSEKIEEQGQKIDRIEDKV
SKAKEHVEKGVEDTKKAVKYQSKARRKKMMVVVVVVVVVVVVVVVVYF
> SEQ ID NO: 143
GELKEKKEKLSKEFEKLLKESKRLAEELKEKLEELGRALDEAEELADEVERQQEELEKLQEEILKSEENEDE
KKQLEELEKKLKELEELLKELEEKLKEVEELMKEVEELMEELEKTMEEMEKAIEELEKVYKEELKKTEAKLK
ATKAEAEAAKAKGEDISDKLEAAEKEYKSVKEELKLVEEIKKKVEEIKEMLEEMKERIEEMEEKVKRIEEKL
KRIEESLKRVEENLKEIKELAKKREEKGIMIIICCVILGIVIASTVGGIFG
> SEQ ID NO: 144
DELEKKIKELEEKSEEELKEAKELAEELRRLLEELERALDEAERLADEVERKQEELEKLMEEMLKSEDNESD
EEDLKKLKEKLEELEKLLEELEERAREVEELMERVEETMEELEEEMEELLETLKKLLEVYEELLKKKKKELE
ETKKKAEEMKKKGIDISEELEKAKEELESVKKNLELVKKILEEVKEIKEELEEMGEEIERMEEKVDRIEEKL
ERVEESLERVSKNLEEIERLFEERKEKGIMIIICCVILGIVIASTVGGIFG
> SEQ ID NO: 145
SEEDKKMEELLEEALKLLEELKELLEKNRELLEELGRQQEELEKLQDEAERLQEELEEAFKKMEENEESEEG
KKYLEEAEKLLKELKKLLEEIEKKTKEIEELVKKQEELMKKIKEVMKKLEEKMKELYRISKERLERAKEEAA
RAEAARAEYEAAGSPEVERAEQVLEEYREAKEFYEKVEELLREVKEIKEEIKEMEERIKEIGERIKRIEEKI
ERVEKLLERTEKNIKEIKELRDKIEKNGIMIIICCVILGIVIASTVGGIFG
> SEQ ID NO: 146
SEKEKEFNELLEEALRELEKLKELLEENGRLLERTGEQLERMEELMDEAEEKQEELEEAIKKMEKYEDSEEG
DEYLEEAEELLEELEELLEEIEAQTEEIEALIKEQEELMKKIKEEMEKLKEAVEKLYEISKEMLEEAKKEYE
KAEKAKAEYEAAGKDEVKECEKVKEKYEEAKKRYEQVEKLLKEVEEIKEEIERMGEEIKRQGERIERIEEKI
ERVEEELERLEENLEEIKKLREKIKENGIMIIICCVILGIVIASTVGGIFN
1^{st} Coiled-coil domain (Bold font) (corresponds to C-terminal coiled-coil domain of native SNAP25, the sequence is either identical to native SNAP25 or redesigned)
2^{nd} Coiled-coil domain: (Bold font) (corresponds to N-terminal coiled-coil domain of native SNAP25 but the sequence is redesigned)
3^{rd} Coiled-coil domain: (Bold font) (corresponds to native Synla coiled- coil domain but the sequence is either identical to native Syn1A or redesigned)
JMD: (underlined)
TMD: 22 aa (italicized)
*GS linkers between coiled-coil domains can be modified to any appropriate linker)

TABLE 16

| # | amino acid sequence |
|---|---|
| N9 | KPGEEKLNKLLEELLKKLEELKKLAEENRRLLERQGROLEELERRFEELNRRMEELNEKLEKLLKEEPNEE |
| | TGEKLEEIKKELEELSRELKELEERVRRQEEEHERQREVVEEIKKELEEAKKYCEELLKTSEEILEEMLEN |
| | PKPYTPEELEKVRERHELIKKLLEEIEELEEMFEELERLVEEQGRRLERIEEKVSRAVRHVERAEENLGEA |
| | VEYLEKSKKLEIMIIICCVILGIVIASTVGGIFA SEQ ID NO: 446 |
| N10 | EPKEKELEELLEELLRELEEIKKLLEEFRRLQEEIGRQIEEIERQLEELLERLEELNEKLENLLKREDNEN |
| | DLEELKELLEEMRELGREMRELERRVEELGRLLEEQRRLVEELKKKLERLLELVKRLLELVEEILEEMLEN |
| | PKPYTPEELEKVRERHELIKKLLEEIEELEEMFEELERLVEEQGRRLERIEEKVSRAVRHVERAEENLGEA |
| | VEYLEKSKKLEIMIIICCVILGIVIASTVGGIFA SEQ ID NO: 447 |
| N11 | RPEEEKLNELLDELLRLLEEIKKLLEENRALLEEIGROIDRIEEQLDRLLRELKELNEKLEALLKREDNEN |
| | DLEELKELLEEIKRLSEEMKELEREVERLGELLEEQRRKVEELKRKLEELLELTEEALELVEEILEEMLEN |
| | PKPYTPEELEKVRERHELIKKLLEEIEELEEMFEELERLVEEQGRRLERIEEKVSRAVRHVERAEENLGEA |
| | VEYLEKSKKLEIMIIICCVILGIVIASTVGGIFA SEQ ID NO: 448 |
| N12 | SLEEILEKLKEIAELLEEVEELTEELKEETERAGRELEELERRLEELVRRAEELNRKLEKILEEEDSDDIL |
| | ERLKEARRELRELRERLEEVEREIERLIREAEEQSELLEELERELEEIKELLKELLEKEEELSEEELELIK |
| | KLLEEIEELEEMFEELERLVEEQGRRLERIEEKVSRAVRHVERAEENLGEAVEYLEKSKKLEIMIIICCVI |
| | LGIVIASTVGGILA SEQ ID NO: 449 |
| N28 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSGGSGGSDARENEMDENLEQVSGIIGNLRHMALDMGNEIDT |
| | QNRQIDRIMEKADSAKTRIDEANQRATKMLGSGGGSGGSEELKKLEKEGEKLKELVEELDREIKELKE |
| | GMERLREMFEEAAKLSEEALEIMRRTRKLSEEELEEAKOKAKEQEKEKALKEKGGSGGSGGSEKVKRE |
| | LAQIEERHQQILELEEKIKELLEMFKELSEKIEEQGQKIDRIEDKVSKAKEHVEKGVEDTKKAVKYQS |
| | KARRKKIMIIICCVILGIVIASTVGGIFA SEQ ID NO: 450 |
| N29 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVFRRISGGSGGSGGSGGSGGSGGSGGSGGSDARENEMDENLEQVSGIIGNLRH |
| | MALDMGNEIDTQNRQIDRIMEKADSAKTRIDEANQRATKMLGSGGGSGGSEELKKLEKEGEKLKELVE |
| | ELDREIKELKEGMERLREMFEEAAKLSEEALEIMRRTRKLSEEELEEAKOKAKEQEKEKALKEKGGSG |
| | GSGGSEKVKRELAQIEERHOQILELEEKIKELLEMFKELSEKIEEQGQKIDRIEDKVSKAKEHVEKGV |
| | EDTKKAVKYQSKARRKKIMIIICCVILGIVIASTVGGIFA SEQ ID NO: 451 |
| N30 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSGGSGGSSEREKEIDEGLDRVSEIVKELKKMAEEMRRMIEE |
| | QGRRIERIEEKAEEAKEKIEEANERAEKLLKDPGGSGGSEELKKLEKEGEKLKELVEELDREIKELKE |
| | GMERLREMFEEAAKLSEEALEIMRRTRKLSEEELEEAKQKAKEQEKEKALKEKGGSGGSGGSEKVKRE |
| | LAQIEERHOQILELEEKIKELLEMFKELSEKIEEQGQKIDRIEDKVSKAKEHVEKGVEDTKKAVKYQS |
| | KARRKKIMIIICCVILGIVIASTVGGIFA SEQ ID NO: 452 |
| N31 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSGGSGGSGGSGGSGGSGGSSEREKEIDEGLDRVSEIVKELKK |
| | MAEEMRRMIEEQGRRIERIEEKAEEAKEKIEEANERAEKLLKDPGGSGGSEELKKLEKEGEKLKELVE |
| | ELDREIKELKEGMERLREMFEEAAKLSEEALEIMRRTRKLSEEELEEAKOKAKEQEKEKALKEKGGSG |
| | GSGGSEKVKRELAQIEERHQQILELEEKIKELLEMFKELSEKIEEQGQKIDRIEDKVSKAKEHVEKGV |
| | EDTKKAVKYQSKARRKKIMIIICCVILGIVIASTVGGIFA SEQ ID NO: 453 |
| N32 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSSEREKEIDEGLDRVSEIVKELKKMAEEMRRMIEEQGRRIE |
| | RIEEKAEEAKEKIEEANERAEKLLKDPGGSGGSEELKKLEKEGEKLKELVEELDREIKELKEGMERLR |
| | EMFEEAAKLSEEALEIMRRTRKLSEEELEEAKQKAKEQEKEKALKEKGGSGGSGGSEKVKRELAQIEE |
| | RHQQILELEEKIKELLEMFKELSEKIEEQGQKIDRIEDKVSKAKEHVEKGVEDTKKAVKYQSKARRKK |
| | IMIIICCVILGIVIASTVGGIFA SEQ ID NO: 454 |
| N33 | VAILWHEMWHEGLEEASRLYFGERNVKGMFEVLEPLHAMMERGPQTLKETSFNQAYGRDLMEAQEWCRKYM |
| | KSGNVKDLTQAWDLYYHVERRISGGSGGSSEKEKEIDELLDKVSEIVKELKKLAEELKRRTERQGRQIE |
| | EIERKTEEAKRKIEELNKKAEELLKKEDDDSDLEKTKELLKEAKEQLREVKEIKRRVEELKREQEETL |
| | KLTKEAAELAEEAKELMEEMLELSEEILEEMLENPKPYTPEELEKVRERHELIKKLLEEIEELEEMFE |
| | ELERLVEEQGRRLERIEEKVSRAVRHVERAEENLGEAVEYLEKSKKLEIMIIICCVILGIVIASTVGG |
| | IFA SEQ ID NO: 455 |

The nucleic acids of all aspects of the disclosure may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptide or fusion protein of the disclosure. 10

The expression vectors of all aspects of the disclosure comprise the nucleic acid of any aspect of the disclosure operatively linked to a suitable control sequence, such as a promoter. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In a fifth aspect, the disclosure provides polypeptides or fusion proteins encoded by the nucleic acid of any embodiment herein.

In a sixth aspect, the disclosure provides host cells comprising the nucleic acid, expression vector, polypeptide, and/or fusion protein of any embodiment or combination of embodiments herein. In one embodiment, the host cell comprises a membrane fusion protein complex anchored in a lipid bilayer membrane of the cell, wherein the membrane fusion protein complex comprises the following components:
(a) a polypeptide encoded by the nucleic acid of embodiment of the first aspect of the disclosure (VAMP2 redesign/v-SNARE-like); and
(b) a polypeptide encoded by the nucleic acid of any embodiment of the second aspect of the disclosure (SNAP25 redesigns); and
(c) a polypeptide encoded by the nucleic acid of any embodiment of the third aspect of the disclosure (Syn1A redesigns);
wherein components (a)-(c) form a hetero-oligomeric complex anchored in a lipid bilayer membrane of the cell, and wherein the hetero-oligomeric complex is capable of inducing membrane fusion.

As described in the examples, the disclosure provides a series of membrane fusion proteins that can induce cell-cell fusion when expressed on the surface of mammalian cells, or liposome fusion when displayed on the surface of liposomes. The designed proteins are based on the human neuronal SNARE complex (which is composed of three proteins, VAMP2, Syntaxin 1A or Syn1A, and SNAP25), which has a parallel four-helical bundle structure and transmembrane domains at the C-terminus of VAMP2 and Syn1A (see FIG. 1). VAMP2 is called v-SNARE, and Syn1A and SNAP25 are called t-SNARE since they exist on the vesicle (v-) or target (t-) membrane inside cells. The four alpha helices of the SNARE complex are composed of one helix from each of VAMP2 and Syn1A and two helices from SNAP25. The nucleic acids of this aspect encode the helix (X1 domain) and membrane domains (X2 and X3) of redesigned VAMP2 (hereafter v-SNARE-like proteins or v-SPs). For membrane fusion to occur, v-SP is presented on the surface of one membrane and SNAP25 redesign and Syn1A redesign on the other.

In another embodiment, the host cell comprises a membrane fusion protein complex anchored in a lipid bilayer membrane of the cell, wherein the membrane fusion protein complex comprises the following components:
(a) a polypeptide encoded by the nucleic acid of any embodiment of the first aspect of the disclosure (VAMP2 redesign/v-SNARE-like); and
(b) a polypeptide encoded by the nucleic acid of any embodiment of the fourth aspect of the disclosure (sc-t-SP);
wherein components (a)-(b) form a hetero-oligomeric complex anchored in a lipid bilayer membrane of the cell, wherein the hetero-oligomeric complex is capable of inducing membrane fusion.

As further described in the examples, new sequences were generated that are believed to fold into the four-helix bundle structure like the parental SNARE complex, followed by engineering SNAP25 so that one of the two coiled-coil domains of SNAP25 is an anti-parallel coiled-coil (FIG. 2, first modification). By combining engineered SNAP25 which has one anti-parallel coiled-coil domain and Syn1A into a single protein, a two-component fusion machinery was generated (one v-SP and one t-SNARE, rather than three components of the original neuronal SNARE; FIG. 2, second modification). Based on the single chain t-SNARE (sc-t-SNARE) backbone, dozens of further sequences that are capable of inducing membrane fusion in a mammalian cell-cell fusion assay (5.s.7-5.s.12 in FIG. 2, third modification) were generated, including those that showed a 10-fold increased fusion efficiency compared to the parental neuronal SNARE complex. These sc-t-SNAREs are designated as single-chain t-SNARE-like proteins or sc-t-SPs. For membrane fusion to occur, v-SP is present on the surface of one membrane and sc-t-SP on the other.

In a seventh aspect, the disclosure provides vesicles, comprising one or more polypeptide or fusion protein of any embodiment herein incorporated into the lipid envelope of the vesicle. The vesicle may be any vesicle that comprises a lipid envelope. In various non-limiting embodiments, the vesicle comprises a liposome, a lipid nanoparticle, a viral vector, or an enveloped particle that may optionally comprise any suitable cargo, including but not limited to a protein or nucleic acid cargo. In some embodiments, one or more polypeptide or fusion protein of any embodiment herein are anchored on a surface of the liposome, the lipid nanoparticle, the viral vector, or the enveloped particle.

All embodiments of the host cells and vesicles disclosed herein may further comprise a therapeutic or diagnostic moiety loaded in the host cell or vesicle. The host cells and vesicles may be used, for example, for intracellular delivery of such therapeutic or diagnostic moieties. Any therapeutic or diagnostic moiety may be loaded into the host cell or vesicle as appropriate for an intended use. In non-limiting embodiments, the therapeutic or diagnostic moiety may comprise a protein or nucleic acid therapeutic or diagnostic moiety.

In an eight aspect, the disclosure provides kits, comprising
(a) a first host cell or vesicle comprising the nucleic acid of any embodiment of the first aspect of the disclosure (VAMP2 redesign/v-SNARE-like); and
(b) a second host cell or vesicle comprising the nucleic acid of any embodiment of the second aspect of the disclosure (SNAP25), and the nucleic acid of any embodiment of the third aspect of the disclosure (Syn1A).

In one embodiment, the first host cell comprises a polypeptide encoded by the nucleic acid of any embodiment of the first aspect of the disclosure (VAMP2 redesign/v-SNARE-like) anchored in a lipid bilayer membrane of the cell or vesicle. In another embodiment, the second host cell comprises a polypeptide encoded by the nucleic acid of any embodiment of the third aspect of the disclosure (Syn1A) anchored in a lipid bilayer membrane of the cell or vesicle.

In a ninth aspect, the disclosure provides kits comprising
(a) a first host cell or vesicle comprising the nucleic acid of any embodiment of the first aspect of the disclosure (VAMP2 redesign/v-SNARE-like); and
(b) a second host cell or vesicle comprising polypeptide encoded by the nucleic acid of any embodiment of the fourth aspect of the disclosure (sc-t-SP).

In one embodiment, the first host cell or vesicle comprises a polypeptide encoded by the nucleic acid of any embodiment of the first aspect of the disclosure (VAMP2 redesign/v-SNARE-like) anchored in a lipid bilayer membrane of the cell or vesicle. In another embodiment, the second host cell or vesicle comprises a polypeptide encoded by the nucleic acid of any embodiment of the fourth aspect of the disclosure anchored in a lipid bilayer membrane of the cell or vesicle.

In all embodiments of the kits of the disclosure, the first host cell or vesicle and/or the second host cell or vesicle may further comprise a therapeutic or diagnostic moiety loaded in the cell or vesicle, as described herein.

In a tenth aspect, the disclosure provides methods for inducing membrane fusion, comprising mixing:
(a) a first host cell or vesicle comprising a polypeptide encoded by the nucleic acid of any embodiment of the first aspect of the disclosure (VAMP2 redesign/v-SNARE-like) anchored in a lipid bilayer membrane of the cell or vesicle; and
(b) a second host cell or vesicle comprising a polypeptide encoded by the nucleic acid of any embodiment of the third aspect of the disclosure (Syn1A) anchored in a lipid bilayer membrane of the cell; wherein the polypeptide encoded by the nucleic acid of any embodiment of the third aspect of the disclosure (Syn1A) is non-covalently bound to a polypeptide encoded by the nucleic acid of any embodiment of the second aspect of the disclosure (SNAP25);
under conditions to promote fusion of the first host cell or vesicle and the second host cell or vesicle.

In another embodiment, the methods for inducing membrane fusion comprise mixing:
(a) a first host cell or vesicle comprising a polypeptide encoded by the nucleic acid of any embodiment of the first aspect of the disclosure (VAMP2 redesign/v-SNARE-like) anchored in a lipid bilayer membrane of the cell or vesicle; and
(b) a second host cell or vesicle comprising a polypeptide encoded by the nucleic acid of any embodiment of the fourth aspect of the disclosure (sc-t-SP);
under conditions to promote fusion of the first host cell or vesicle and the second host cell or vesicle.

In one embodiment, the methods comprise first delivering the nucleic acids or expression vectors of the disclosure into target cells in vivo by a conventional delivery system (viral vector, etc.) so that the target cell becomes a first host cell as recited above, and then, delivering therapeutic or other moiety to the target cell by using a vesicle that is a second vesicle as described above. In another embodiment, the methods comprise first delivering the nucleic acids of the disclosure into target cells in vivo by a conventional delivery system (viral vector, etc.) so that the target cell becomes a second host cell as recited above, and then, delivering therapeutic or other moiety to the target cell by using a vesicle that is a first vesicle as described above

The host cells of the disclosure may comprise the polypeptide, fusion protein nucleic acid and/or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection. In some embodiments, the cells are eukaryotic cells comprising lipid bilayers, such as mammalian cells including but not limited to human cells.

### Examples

We made a series of membrane fusion proteins (see Tables above) that can induce cell-cell fusion when expressed on the surface of mammalian cells, or liposome fusion when displayed on the surface of liposomes. The human neuronal SNARE complex (which is composed of three proteins, VAMP2, Syntaxin 1A or Syn1A, and SNAP25) has a parallel four-helical bundle structure and transmembrane domains at the C-terminus of VAMP2 and Syn1A (see FIG. 1). VAMP2 is called v-SNARE and Syn1A and SNAP25 are called t-SNARE since they exist on the vesicle (v-) or target (t-) membrane inside cells. The four alpha helices of the SNARE complex are composed of one helix from each of VAMP2 and Syn1A and two helices from SNAP25.

We first redesigned the amino acid sequence of the human neuronal SNARE and generated new sequences that are likely to fold into the four-helix bundle structure like the parental SNARE complex. Next, we engineered SNAP25 so that one of the two coiled-coil domains of SNAP25 is an anti-parallel coiled-coil (FIG. 2, first modification). By combining engineered SNAP25 which has one anti-parallel coiled-coil domain and Syn1A into a single protein, we successfully generated the two-component fusion machinery (one v-SNARE and one t-SNARE, rather than three components of the original neuronal SNARE; FIG. 2, second modification). Based on the single chain t-SNARE (sc-t-SNARE) backbone, we further generated dozens of new sequences that are capable of inducing membrane fusion in a mammalian cell-cell fusion assay (5.s.7-5.s.12 in FIG. 2, third modification). Some of the best designs showed over 10-fold increased fusion efficiency compared to the parental neuronal SNARE complex. Hereafter, redesigned VAMP2 proteins and sc-t-SNARE proteins are designated as v-SNARE-like proteins (v-SPs) and sc-t-SNARE-like proteins (sc-t-SPs), respectively.

Furthermore, we have generated new protein backbones based on the structure of sc-t-SP and made new sequences for these backbones. These sequences are likely to fold into SNARE complex-like structures, but their predicted structures are slightly different from the original neuronal SNARE (single-digit RMSD). These new proteins also showed significantly higher fusion activity compared to native neuronal SNARE.

Our studies demonstrated that the juxtamembrane domain (JMD) of native VAMP2 and v-SP is important for activity, but various non-native sequences (K9, KIF, and RIF) showed substantial fusion activity. Our studies further demonstrated that the transmembrane domain of native VAMP2, v-SPs, native t-SNAREs, and sc-t-SPs can be replaced with non-native sequences, including TMD derived from VSV-G, flu HA, EGFR, PDGFR, and non-cognate SNARE (like VAMP2 protein with Syn1A-TMD or vice versa). Finally, while Syn1A JMD (TKKAVKYQSKARRKK, (SEQ ID NO: 331)) is critical for fusion activity in native three-component fusion machinery, in our designs, this JMD sequence is not essential for activity and can be replaced with non-native sequences, as shown in various sc-t-SP designs disclosed herein.

We describe designed proteins that have membrane fusion activity, and are useful, for example, in intracellular delivery and synthetic intracellular membrane trafficking systems.

Computationally designed amino acid sequences that fold into a SNARE complex-like four-helix bundle structure and are capable of inducing the fusion of two membranes when displayed on the lipid bilayer membrane such as cell membrane and liposomal membrane. The designed protein complex is composed of two (SEQ ID: 64-146) or three (SEQ ID: 38-63) protein components and anchored into the membrane by their transmembrane domains.

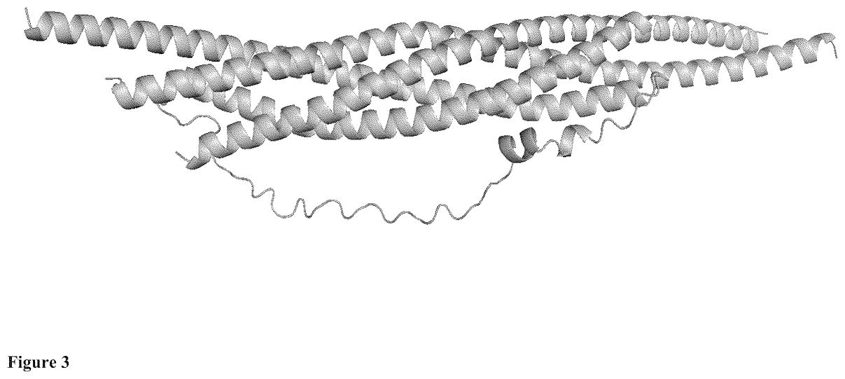

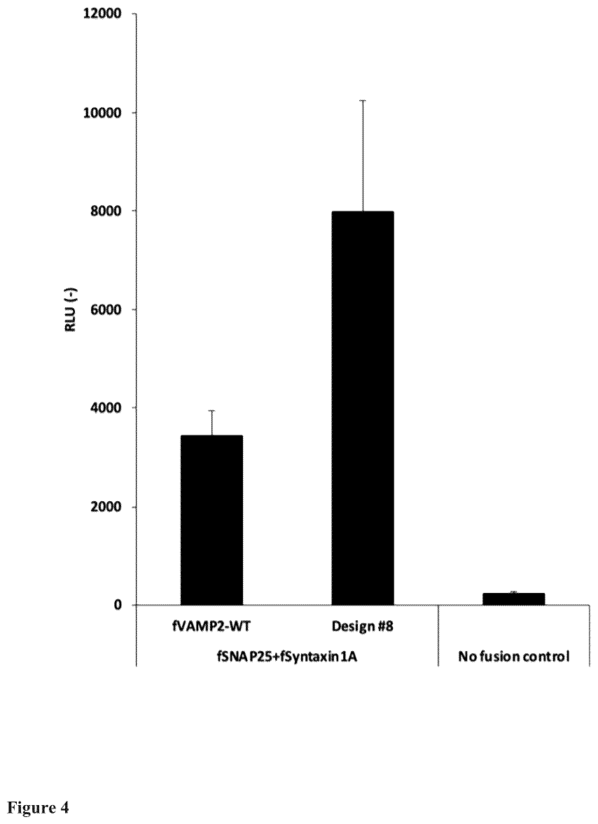

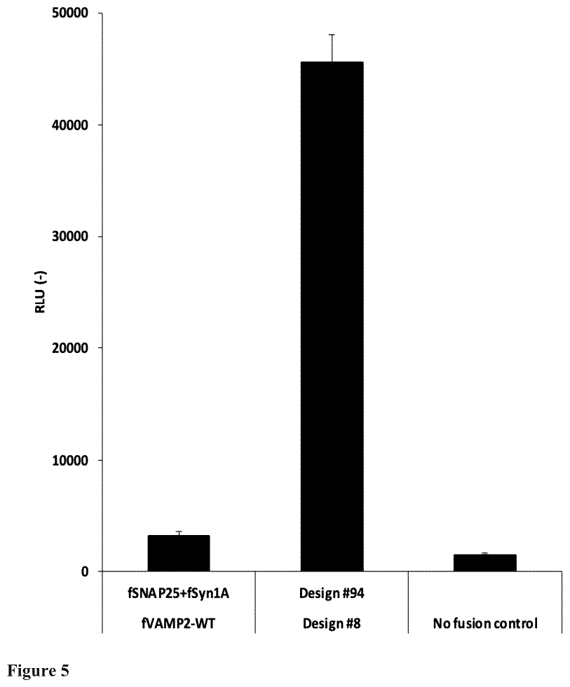

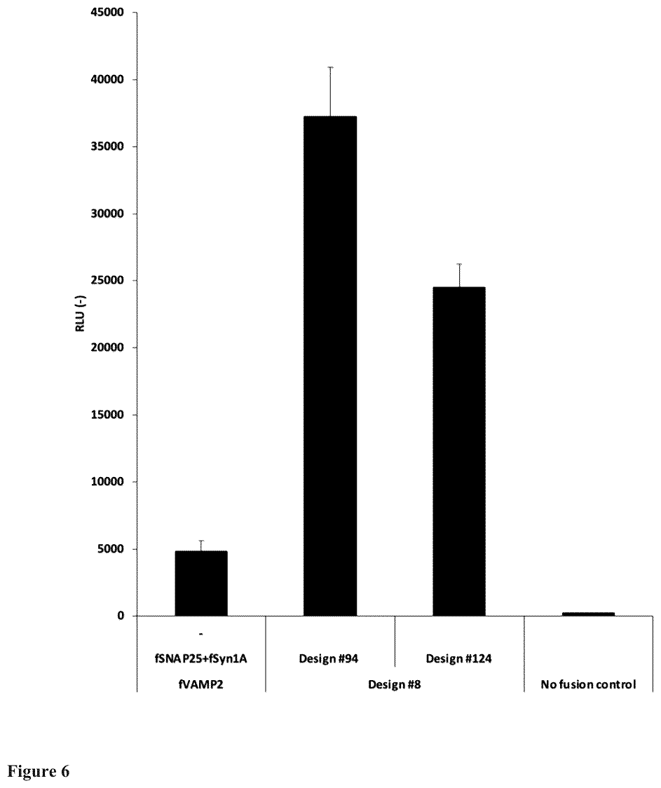

The new sequences were designed using the native SNARE structure as a template (SEQ ID: 1-63, except 26-33, 42-44, and 58-63). The predicted structure of these designs is identical to that of the parental SNARE complex (FIG. 3). One of the v-SNARE-like proteins (v-SP), SEQ ID 8, showed improved fusion activity compared to native VAMP2 in cell-cell fusion assay (FIG. 4). While native neuronal SNARE is composed of three protein components (one v-SNARE and two t-SNAREs), we generated a single-chain t-SNARE-like protein (sc-t-SP) by flipping the backbone of one helix from the coiled-coil domain of SNAP25 and genetically fusing the C terminus of SNAP25 to the N terminus of Syntaxin 1A (SEQ ID: 64-65). By making truncation mutants, some part of the sc-t-SP was found to be indispensable for fusion activity (SEQ ID: 66-74). Further sequence redesign led to the creation of new proteins that have over 10-fold increased fusion activity compared to the parental SNARE complex (FIG. 5) (SEQ ID: 75-122). We then removed the 20 N-terminal amino acids from sc-t-SP that are dispensable and further redesigned the sequence (SEQ ID: 123-127). These shorter designs showed comparable activity to the longer sc-t-SP (FIG. 6). These synthetic fusogens can induce fusion of liposome membrane.

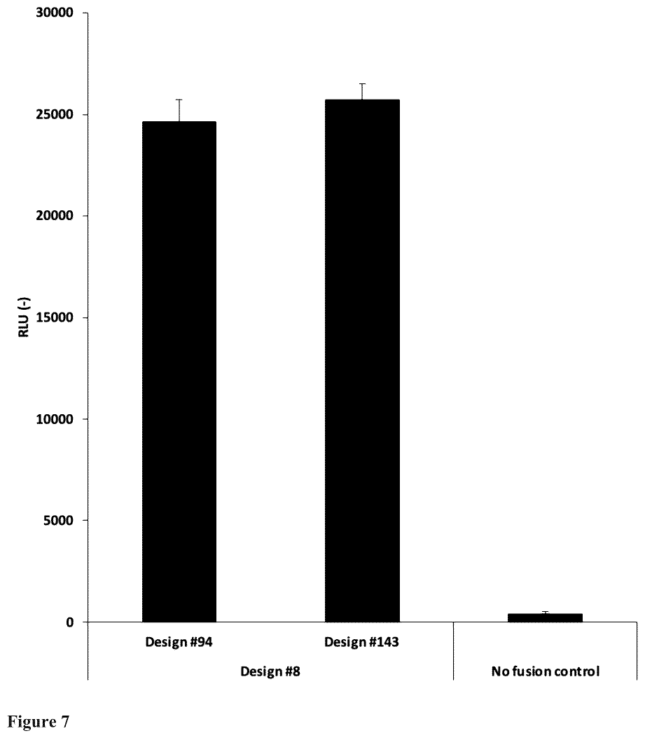

Furthermore, we generated the new backbone of sc-t-SP. The structure of parental neuronal SNARE was “partially diffused” by RFdiffusion and the newly generated backbones and sequences were predicted to fold into SNARE complex-like four-helix bundle structures (SEQ ID: 128-146). These designs showed superior fusion activity compared to the parental native SNARE complex (FIG. 7).

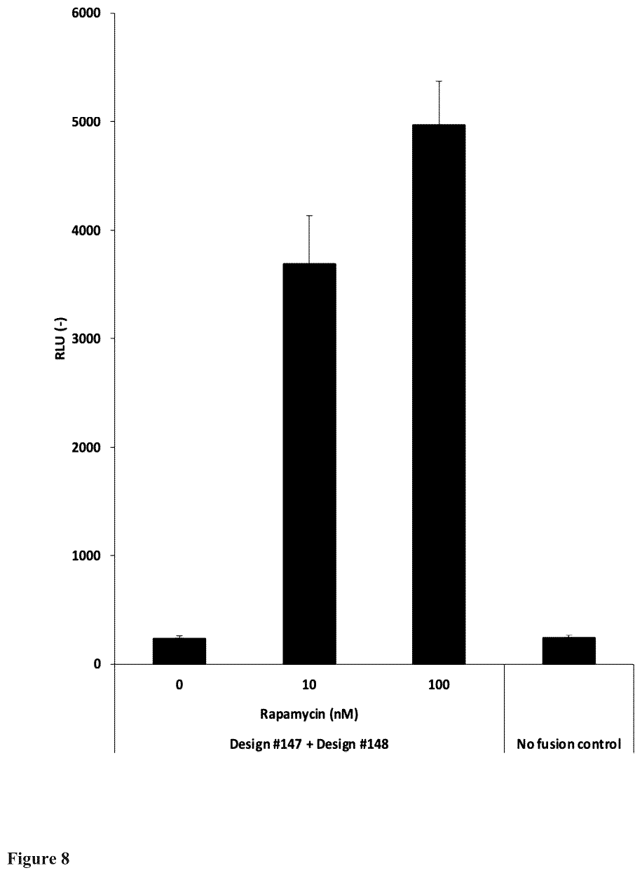

When combined with small molecule-dependent heterodimeric domains, the fusogenic activity of these designed fusion proteins can be controlled by the presence of specific small molecules (chemically induced dimerization; exemplified by rapamycin induced binding herein FIG. 8) (SEQ ID: 147-148).

### Materials and Methods

Genes for designed proteins were synthetized and cloned into mammalian expression vectors such as pCMV or pcDNA3.1. All designs were expressed in human embryonic kidney cell line HEK293T by transfection of plasmid DNA using polyethyleneimine (PEI). Designed SNARE-like proteins were expressed on the surface of HEK293T cells as flipped SNARE³. The v-cells express v-SP and T7 RNA polymerase while t-cells express t-SP and reporter luciferase under T7 promoter. In this assay, only after the cell-cell fusion between v-cells and t-cells, reporter luciferase gene is expressed. Transfected cells were mixed together and after overnight incubation, cell-cell fusion was quantitatively assessed by luciferase assay.

### REFERENCES

1. J. Dauparas et al., “Robust deep learning-based protein sequence design using ProteinMPNN,” Science, vol. 378, no. 6615, pp. 49-56, September 2022.
2. J. L. Watson et al., “De novo design of protein structure and function with RFdiffusion,” Nature, vol. 620, no. 7976, pp. 1089-110 August 2023.
3. C. Hu, M. Ahmed, T. J. Melia, T. H. Söllner, T. Mayer, and J. E. Rothman, “Fusion of Cells by Flipped SNAREs,” Science, vol. 300, no. 5626, pp. 1745-1749 June 2003.

1. A nucleic acid encoding a polypeptide comprising the formula X1-X2-X3, wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of the bold font residues in SEQ ID NO:149-208;
X2 comprises a juxtamembrane domain (JMD), wherein X2 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:500-505; and
X3 comprises a transmembrane domain (TMD).

2. The nucleic acid of claim 1, wherein X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:156.

3. The nucleic acid of claim 2, wherein 1, 2, 3, 4, 5, 6, 7, 8, 9, 10, 11, 12, or all 13 of L8, L12, V15, V18, 121, M22, L28, V29, G33, 136, L39, L46, L53 are conserved (i.e., identical) in the polypeptide relative to SEQ ID NO:156.

4. The nucleic acid of claim 1, wherein X2 comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:209-222 and 456.

5. (canceled)

6. The nucleic acid of claim 1, wherein X3 comprises an amino acid at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: SEQ ID NO:223-234.

7. (canceled)

8. The nucleic acid of claim 1, encoding a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:1-37, 147, and 236-289.

9. The nucleic acid of claim 8, encoding a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8.

10. The nucleic acid of claim 9 wherein 1, 2, 3, 4, 5, 6, 7, 8, 9, 10, 11, 12, or all 13 of L8, L12, V15, V18, 121, M22, L28, V29, G33, 136, L39, L46, L53 are conserved (i.e., identical) in the polypeptide relative to SEQ ID NO:8.

11. (canceled)

12. The nucleic acid of claim 11, encoding a fusion protein comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:147 and 244-258.

13-15. (canceled)

16. A nucleic acid encoding
(I) a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:310-316, wherein X1 is an amino acid linker; or
(II) a polypeptide comprising the formula X1-X2-X3, wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 317-330;
X2 comprises a juxtamembrane domain (JMD); and
X3 comprises a transmembrane domain (TMD).

17-18. (canceled)

19. The nucleic acid of claim 16, encoding a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:38-44.

20-26. (canceled)

27. The nucleic acid of claim 16, encoding a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:45-63.

28-30. (canceled)

31. A nucleic acid encoding a polypeptide comprising the formula X1-X2-X3, wherein
X1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of the first set bold font residues in SEQ ID NO: 333-425, wherein the non-highlighted residues are amino acid linkers that may be substituted with any other amino acid linker;
X2 comprises a juxtamembrane domain (JMD); and
X3 comprises a transmembrane domain (TMD).

32-34. (canceled)

35. The nucleic acid of claim 31, encoding a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:64-146, 148, and 446-455.

36-40. (canceled)

41. A host cell, wherein the host cell comprises a membrane fusion protein complex anchored in a lipid bilayer membrane of the cell, wherein the membrane fusion protein complex comprises the following components:
(I) (a) a polypeptide encoded by the nucleic acid of claim 1; and
(b) a polypeptide encoded by the nucleic acid of any embodiment of the second aspect of the disclosure; and
(c) a polypeptide encoded by the nucleic acid of any embodiment of the third aspect of the disclosure;
wherein components (a)-(c) form a hetero-oligomeric complex anchored in a lipid bilayer membrane of the cell, wherein the hetero-oligomeric complex is capable of inducing membrane fusion; or
(II) (a) a polypeptide encoded by the nucleic acid of claim 1; and
(b) a polypeptide encoded by the nucleic acid of any embodiment of the fourth aspect of the disclosure;
wherein components (a)-(b) form a hetero-oligomeric complex anchored in a lipid bilayer membrane of the cell, wherein the hetero-oligomeric complex is capable of inducing membrane fusion.

42. (canceled)

43. A vesicle, comprising one or more polypeptide of claim 1 incorporated into the lipid envelope of the vesicle.

44-46. (canceled)

47. A kit, comprising:
(I) (a) a first host cell or vesicle comprising the nucleic acid of claim 1; and
(b) a second host cell or vesicle comprising the nucleic acid of any embodiment of the second aspect of the disclosure, and the nucleic acid of any embodiment of the third aspect of the disclosure; or
(II) (a) a first host cell or vesicle comprising the nucleic acid of claim 1; and
(b) a second host cell or vesicle comprising polypeptide encoded by the nucleic acid of any embodiment of the fourth aspect of the disclosure.

48-53. (canceled)

54. A method for inducing membrane fusion, comprising mixing:
(I) (a) a first host cell or vesicle comprising a polypeptide encoded by the nucleic acid of claim 1 anchored in a lipid bilayer membrane of the cell; and
(b) a second host cell or vesicle comprising a polypeptide encoded by the nucleic acid of any embodiment of the third aspect of the disclosure anchored in a lipid bilayer membrane of the cell; wherein the polypeptide encoded by the nucleic acid of any embodiment of the third aspect of the disclosure is non-covalently bound to a polypeptide encoded by the nucleic acid of any embodiment of the second aspect of the disclosure; or
(II) (a) a first host cell or vesicle comprising a polypeptide encoded by the nucleic acid of claim 1 anchored in a lipid bilayer membrane of the cell; and
(b) a second host cell or vesicle comprising a polypeptide encoded by the nucleic acid of any embodiment of the fourth aspect of the disclosure;
under conditions to promote fusion of the first host cell or vesicle and the second host cell or vesicle.

55. (canceled)
